(19) United States
(12) Patent Application Publication
(10) Pub. No.: US 2026/0294811 A1
(43) Pub. Date: Oct. 1, 2026

(54) AMINO ACID BACKBONE IONIZABLE LIPID, PREPARATION METHOD THEREOF AND APPLICATION METHOD THEREFOR

(71) Applicant: South China University of Technology, Guangzhou (CN)

(72) Inventors: Yuan ZHANG, Guangzhou (CN); Xiangjie CHEN, Guangzhou (CN)

(21) Appl. No.: 19/670,064

(22) Filed: May 7, 2026

(30) Foreign Application Priority Data
May 9, 2025 (CN) 202510598216.0

(51) Int. Cl. A61K 9/1272; A61K 9/51; A61K 31/7088; C07C 229/24; C07D 207/06; C07D 209/14; C07D 211/14; C07D 233/61; C07D 285/08; C07D 295/15

(52) U.S. Cl. CPC A61K 9/1272; A61K 9/5123; A61K 31/7088; C07C 229/24; C07D 207/06; C07D 209/14; C07D 211/14; C07D 233/61; C07D 285/08; C07D 295/15

(57) ABSTRACT

The present disclosure provides an amino acid backbone ionizable lipid, preparation method thereof and application method therefor. The amino acid backbone ionizable lipid is modified with amino acids as a core, has many ester groups and peptide bonds, and can be quickly hydrolyzed by enzymes after effectively releasing RNA in vivo. The amino acid backbone ionizable lipid has four tail structures, which can increase a cross-sectional area of lipid tails, help RNA and other drugs escape from endosomes, and enhance transfection efficiency. A charge of the ionizable lipid is electrically neutral under physiological conditions, reducing a cytotoxicity caused by excessive positive charges, thereby increasing stability of lipid nanoparticles, helping to prolong circulation time of loaded nucleic acid drugs and improving pharmacokinetic characteristics.

### TECHNICAL FIELD

The present disclosure belongs to the field of drug carrier technology, and specifically relates to an amino acid backbone ionizable lipid, preparation method thereof and application method therefor.

### BACKGROUND

Cancer vaccines generally have four types, including tumor or immune cell vaccines, peptide vaccines, viral vector vaccines, and nucleic acid vaccines. Nucleic acid vaccines are a promising type of vaccine (DNA vaccines or RNA vaccines). Ribonucleic acid (RNA) therapy mainly includes antisense oligonucleotides (ASOs), small interfering RNA (siRNA), small molecule RNA (miRNA), messenger RNA (mRNA), and circular RNA (circRNA), which have shown great potential in treating a wide range of diseases by manipulating different modes of action. Firstly, nucleic acid vaccines can simultaneously deliver multiple antigens such as tumor associated antigens (TAAs) or somatic tumor mutations, triggering humoral and cellular immunity and reducing vaccine resistance. Secondly, nucleic acid vaccines allow APCs to simultaneously or cross present multiple epitopes of class I and class II patient specific Human leukocyte antigens, making them less restricted by human HLA types and more likely to stimulate a wider range of T cell responses. However, due to an inherent negative charge and instability of RNA molecules, it is difficult for RNA to break through biological barriers and reach cytoplasm. To overcome this issue, RNA requires a safe, effective, and stable delivery system to protect the nucleic acid from degradation and accelerate cellular uptake and effective release of RNA. At present, lipid nanoparticles (LNP) are widely used in the field of drug delivery, but RNA vaccines still require more efficient delivery systems.

Various nucleic acid delivery systems can be roughly divided into two categories: viral vectors and non-viral vectors. Where viral vectors have relatively high transfection efficiency, but exist problems such as safety and poor targeting. For decades, liposomes have developed rapidly as a representative non-viral carrier, developing a new type of lipid called ionizable lipid that can be protonated at weak acid pH. Making the ionizable lipid positively charged, but still remain neutral at physiological pH. The pH sensitivity of ionizable lipid is beneficial for in vivo delivery of mRNA, as neutral lipid have less interaction with blood cell anion membranes, improving the biocompatibility of nanoparticles as a result. When lipid nanoparticles are in a weakly acidic pH endosome, ionizable lipid gain charges to promote membrane instability and increase escape of nanoparticles from endosome. However, there are still several aspects of ionizable lipid: (1) toxicity issues: ionizable lipid are key components in lipid nanoparticles (LNP) that trigger acute immune responses and long-term toxicity, but further optimization of lipid structure is still needed; (2) synthesis complexity: a current process of synthesizing ionizable lipid is cumbersome, requiring multiple reactions and complex purification steps, which limits large-scale production and rapid screening; (3) low delivery efficiency: low internal body escape is a common delivery efficiency problem of lipid nanoparticles, and further research is still needed to optimize the internal body escape mechanism.

### SUMMARY

In order to overcome problems existing in the existing technologies, one of objectives of the present disclosure is to provide an amino acid backbone ionizable lipid. The second objective of the present disclosure is to provide a preparation method of the amino acid backbone ionizable lipid. The third objective of the present disclosure is to provide a lipid synthesis. The fourth objective of the present disclosure is to provide an application method of the amino acid backbone ionizable lipid and lipid synthesis in delivery carriers. The fifth objective of the present disclosure is to provide a pharmaceutical synthesis. The sixth objective of the present disclosure is to provide an application method of the amino acid backbone ionizable lipid and the pharmaceutical synthesis in preparing drugs.

The present disclosure proposes an amino acid backbone ionizable lipid, which has a chemical structure consisting of three components: (i) ionizable head group, (ii) linking group, and (iii) hydrophobic tail. The head group is a tertiary or secondary amino group, which can obtain protons at acidic pH and carry positive charges. It can bind with negatively charged nucleic acid molecules or small molecule drugs through electrostatic interactions, and then self assemble with auxiliary lipid to form lipid nanoparticles, thereby delivering gene drugs. A hydrophobic tail have 2-4 more tail groups in a structure compared to commonly used double tailed lipid in existing technology. Due to increased cross-sectional area of tail area, the present type of lipid will produce a more conical structure, having stronger ability to endosomal membrane disruption and enhance delivery efficiency. And based on amino acid synthesis tail, the amino acid backbone ionizable lipid provided by the present disclosure has excellent biosafety. Based on a series of problems encountered in current gene drug delivery, such as low efficiency and high toxicity, the amino acid backbone ionizable lipid balances degradability and ensures lipid safety while maintaining overall delivery efficiency in the chemical structure design. In addition, an preparation method of the amino acid skeleton ionizable lipid of the present disclosure is different from the rigorous and complex synthesis route of traditional cationic lipid. An ionizable lipid library can be obtained through Michael addition, which has advantages of simple synthesis route, clear reaction mechanism, and easy high-throughput screening.

In order to achieve the objectives, a technical solution adopted by the present disclosure is:

A first aspect of the present disclosure provides an amino acid backbone ionizable lipid,

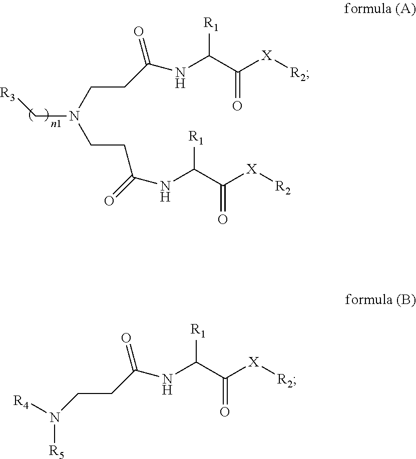

where R₁ is selected from

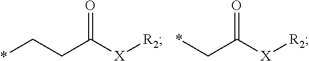

C2-C10 alkyl; C2-C10 heteroalkylene; arylalkyl or non-existence;
R₂ is selected from C3-C24 linear alkyl; C4-C24 linear alkenyl;

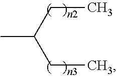

where n1=2-3, n2=3-24, n3=3-24;
X is O or NH;
R₃ is selected from

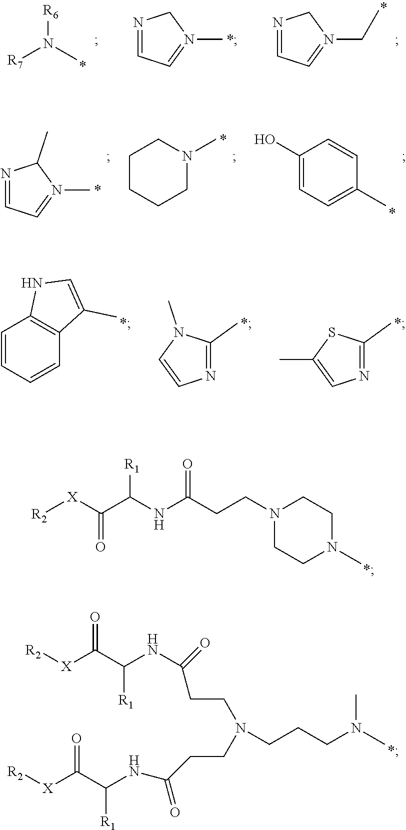

C1-C10 hydroxyalkyl; where R₆ is selected from C1-C4 alkyl, R₇ is selected from C1-C4 hydroxyalkyl;

where R₄ is selected from C1-C4 hydroxyalkyl; R₅ is C1-C4 alkyl, C1-C4 hydroxyalkyl,

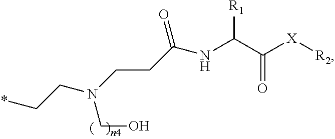

where n4=2-3;
where “*” is a connecting site.

The term “isomer” refers to compounds of the present disclosure that may exist in specific geometric or stereoisomeric forms. The present disclosure envisions all such compounds, including cis isomers and trans isomers, (−)- and (+)-enantiomers, (R)- and (S)-enantiomers, diastereomers, (D)-isomers, (L)-isomers, as well as racemic mixtures and other mixtures, such as enantiomer or diastereomer enriched mixtures, all of which are within the scope of the present disclosure. There may be additional asymmetric carbon atoms in alkyl and other substituents. All of the isomers and mixtures are included within the scope of the present disclosure.

Unless otherwise specified, the terms “enantiomer” or “optical isomer” refer to stereoisomers that are mirror images of each other. Unless otherwise specified, the terms “cis trans isomer” or “geometric isomer” are caused by an inability of double bonds or single bonds of cyclic carbon atoms to rotate freely. Unless otherwise specified, the term “diastereomer” refers to a stereoisomer in which a molecule has two or more chiral centers and is in a non mirror relationship with each other. Unless otherwise specified, “(D)” or “(+)” represents right-handed, “(L)” or “(−)” represents left-handed, and “(DL)” or “(±)” denotes racemic mixtures. Unless otherwise specified, using wedge-shaped solid line key [symbol] and wedge-shaped dotted line key [symbol] to represent an absolute configuration of a solid center.

The term “pharmaceutically acceptable salt” refers to salts of compounds of the present disclosure, prepared from compounds with specific substituents discovered in the present disclosure with relatively non-toxic acids or bases. When the compounds of the present disclosure contain relatively acidic functional groups, base addition salts can be obtained by contacting a sufficient amount of base with a neutral form of such compounds in a pure solution or suitable inert solvent. Pharmaceutically acceptable base addition salts includes sodium, potassium, calcium, ammonium, organic ammonia, magnesium salts, or similar salts. When the compounds of the present disclosure contain relatively basic functional groups, acid addition salts can be obtained by contacting a sufficient amount of acid with a neutral form of such compounds in a pure solution or suitable inert solvent. Examples of pharmaceutically acceptable acid addition salts includes inorganic acid salts, organic acid salts, salts of amino acids such as arginine, as well as salts of organic acids such as glucuronic acid. Where the inorganic acid salts includes hydrochloric acid, hydrobromic acid, nitric acid, carbonic acid, bicarbonate, phosphoric acid, hydrogen phosphate, dihydrogen phosphate, sulfuric acid, hydrogen sulfate, hydroiodate, hypophosphite, etc.; the organic acid salts includes similar acids such as acetic acid, propionic acid, isobutyric acid, maleic acid, malonic acid, benzoic acid, succinic acid, succinic acid, fumaric acid, lactic acid, mandelic acid, phthalic acid, benzenesulfonic acid, p-toluenesulfonic acid, citric acid, tartaric acid, and methanesulfonic acid. Some specific compounds of the present disclosure contain basic and acidic functional groups, which can be converted into any base or acid addition salt.

In some embodiments, a structure of the amino acid backbone ionizes lipid is selected from any one of formula (1) to formula (104):

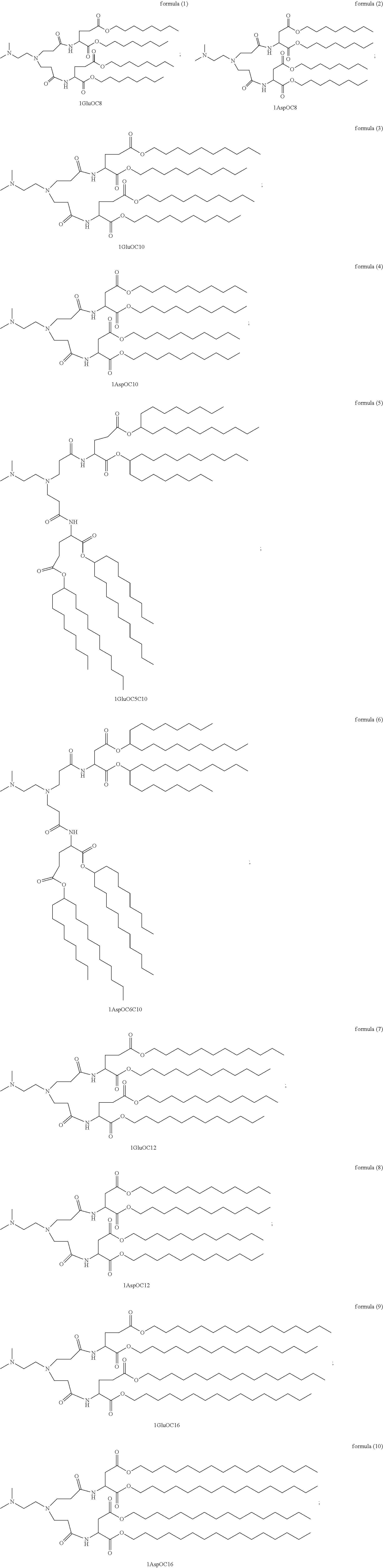

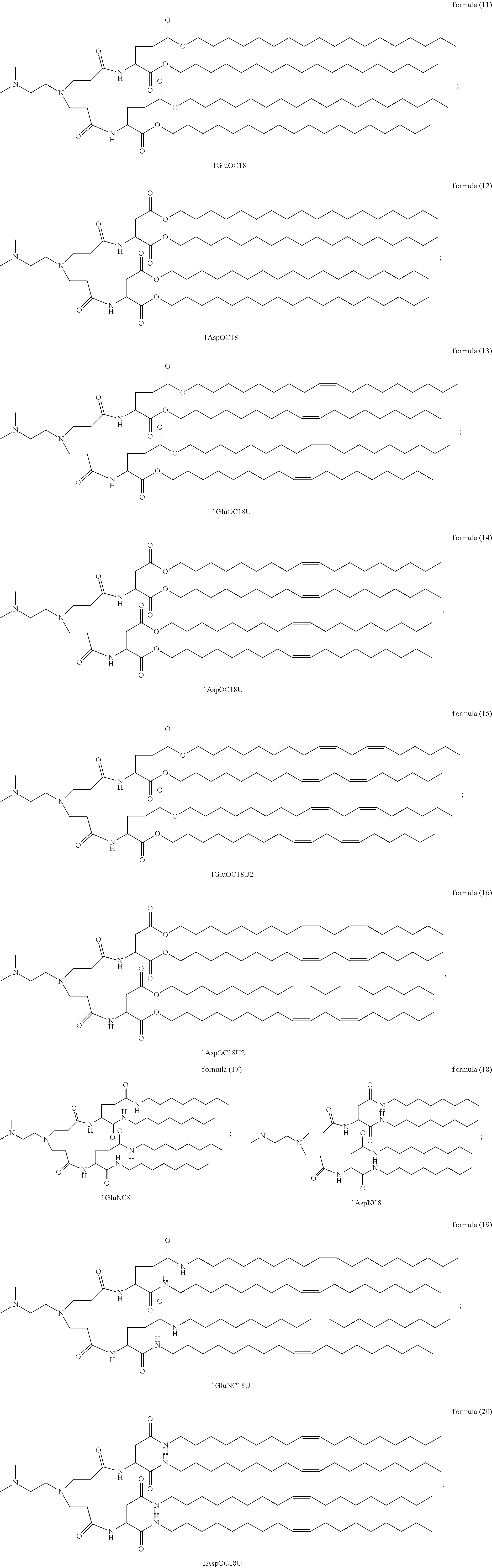

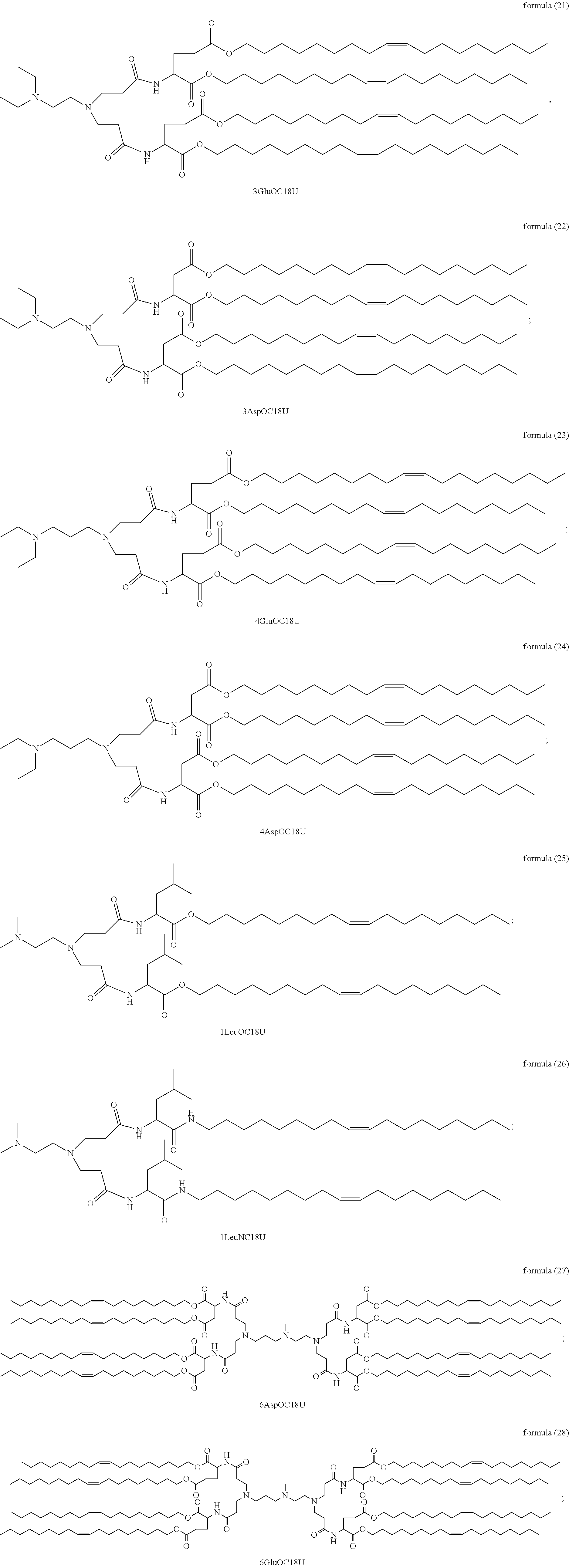

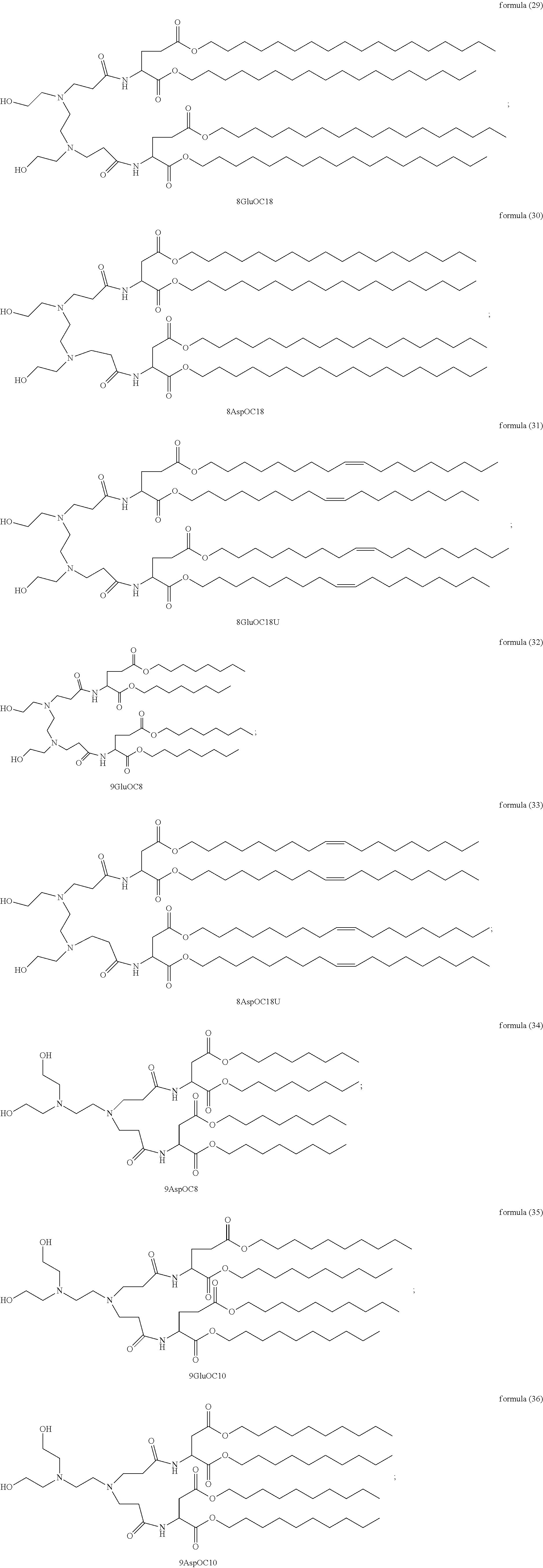

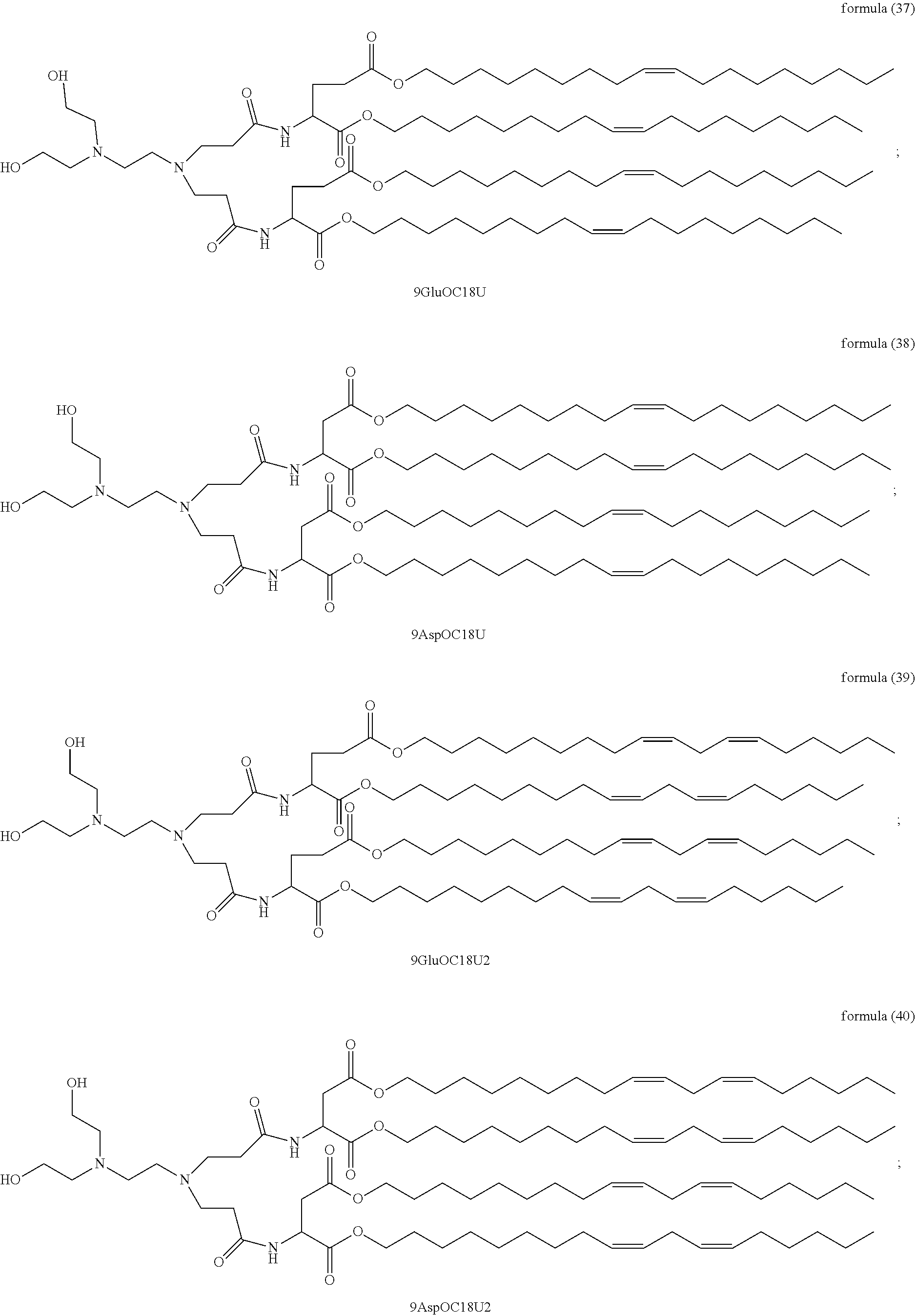

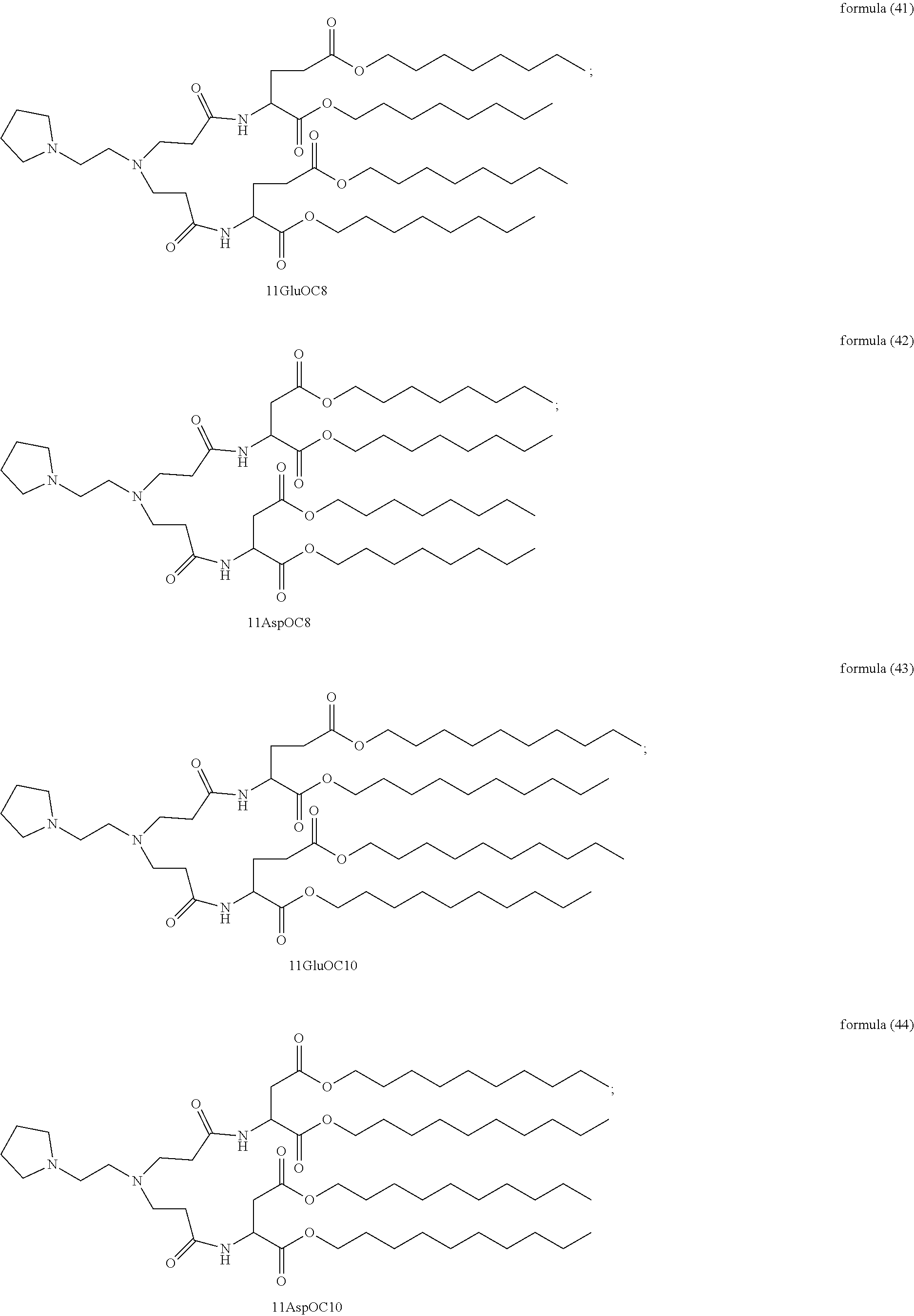 [SMILES: CCCCCCCCOC(=O)CCC(NC(=O)CCN(CCC(=O)NC(CCC(=O)OCCCCCCCC)C(=O)OCCCCCCCC)CCN1CCCC1)C(=O)OCCCCCCCC ; CCCCCCCCOC(=O)CC(NC(=O)CCN(CCC(=O)NC(CC(=O)OCCCCCCCC)C(=O)OCCCCCCCC)CCN1CCCC1)C(=O)OCCCCCCCC ; CCCCCCCCCCOC(=O)CCC(NC(=O)CCN(CCC(=O)NC(CCC(=O)OCCCCCCCCCC)C(=O)OCCCCCCCCCC)CCN1CCCC1)C(=O)OCCCCCCCCCC ; CCCCCCCCCCOC(=O)CC(NC(=O)CCN(CCC(=O)NC(CC(=O)OCCCCCCCCCC)C(=O)OCCCCCCCCCC)CCN1CCCC1)C(=O)OCCCCCCCCCC]

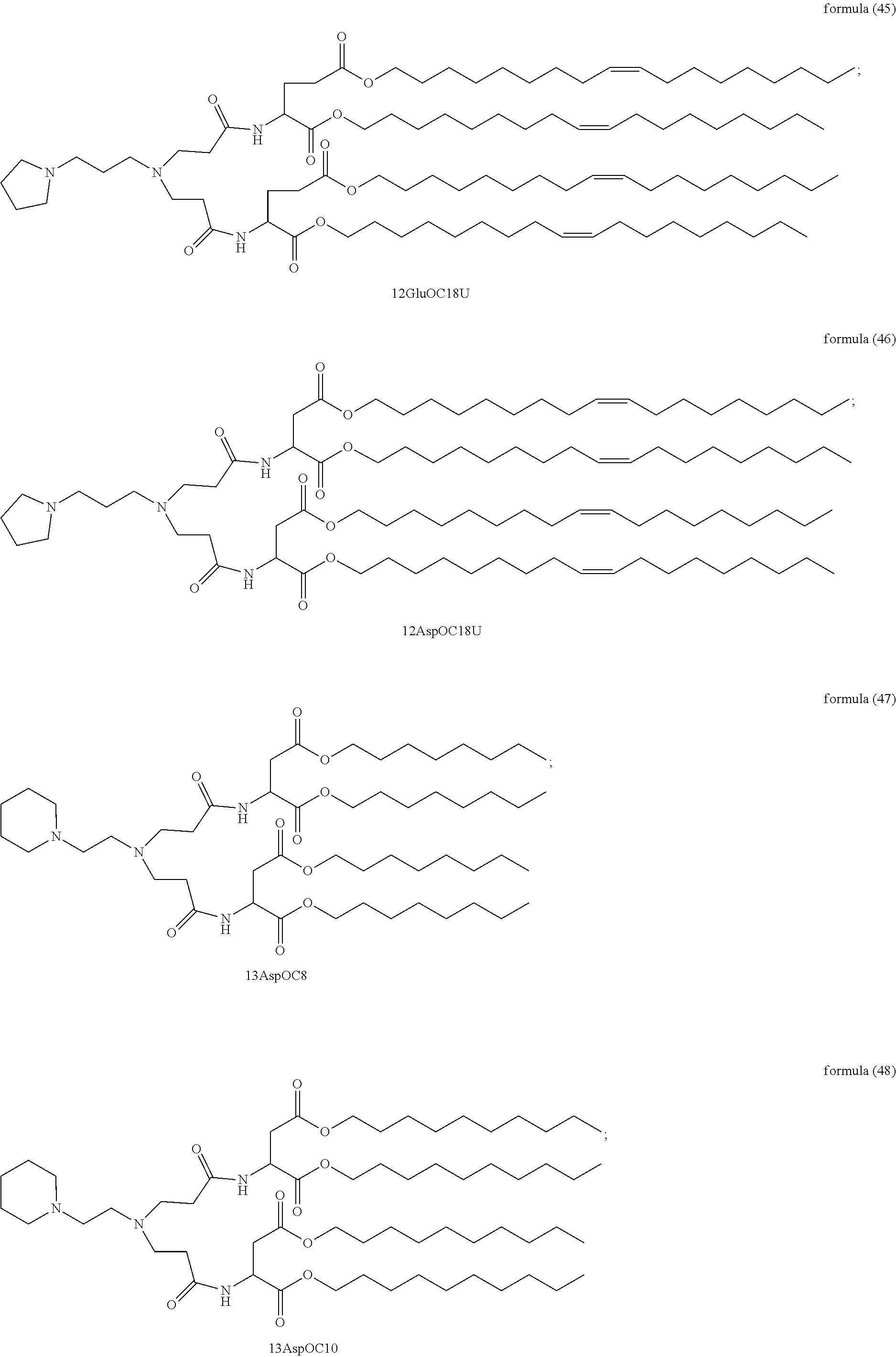 [SMILES: CCCCCCCC/C=C\CCCCCCCCOC(=O)CCC(NC(=O)CCN(CCCN1CCCC1)CCC(=O)NC(CCC(=O)OCCCCCCCC/C=C\CCCCCCCC)C(=O)OCCCCCCCC/C=C\CCCCCCCC)C(=O)OCCCCCCCC/C=C\CCCCCCCC ; CCCCCCCC/C=C\CCCCCCCCOC(=O)CC(NC(=O)CCN(CCCN1CCCC1)CCC(=O)NC(CC(=O)OCCCCCCCC/C=C\CCCCCCCC)C(=O)OCCCCCCCC/C=C\CCCCCCCC)C(=O)OCCCCCCCC/C=C\CCCCCCCC ; CCCCCCCCOC(=O)CC(NC(=O)CCN(CCC(=O)NC(CC(=O)OCCCCCCCC)C(=O)OCCCCCCCC)CCN1CCCCC1)C(=O)OCCCCCCCC ; CCCCCCCCCCOC(=O)CC(NC(=O)CCN(CCC(=O)NC(CC(=O)OCCCCCCCCCC)C(=O)OCCCCCCCCCC)CCN1CCCCC1)C(=O)OCCCCCCCCCC]

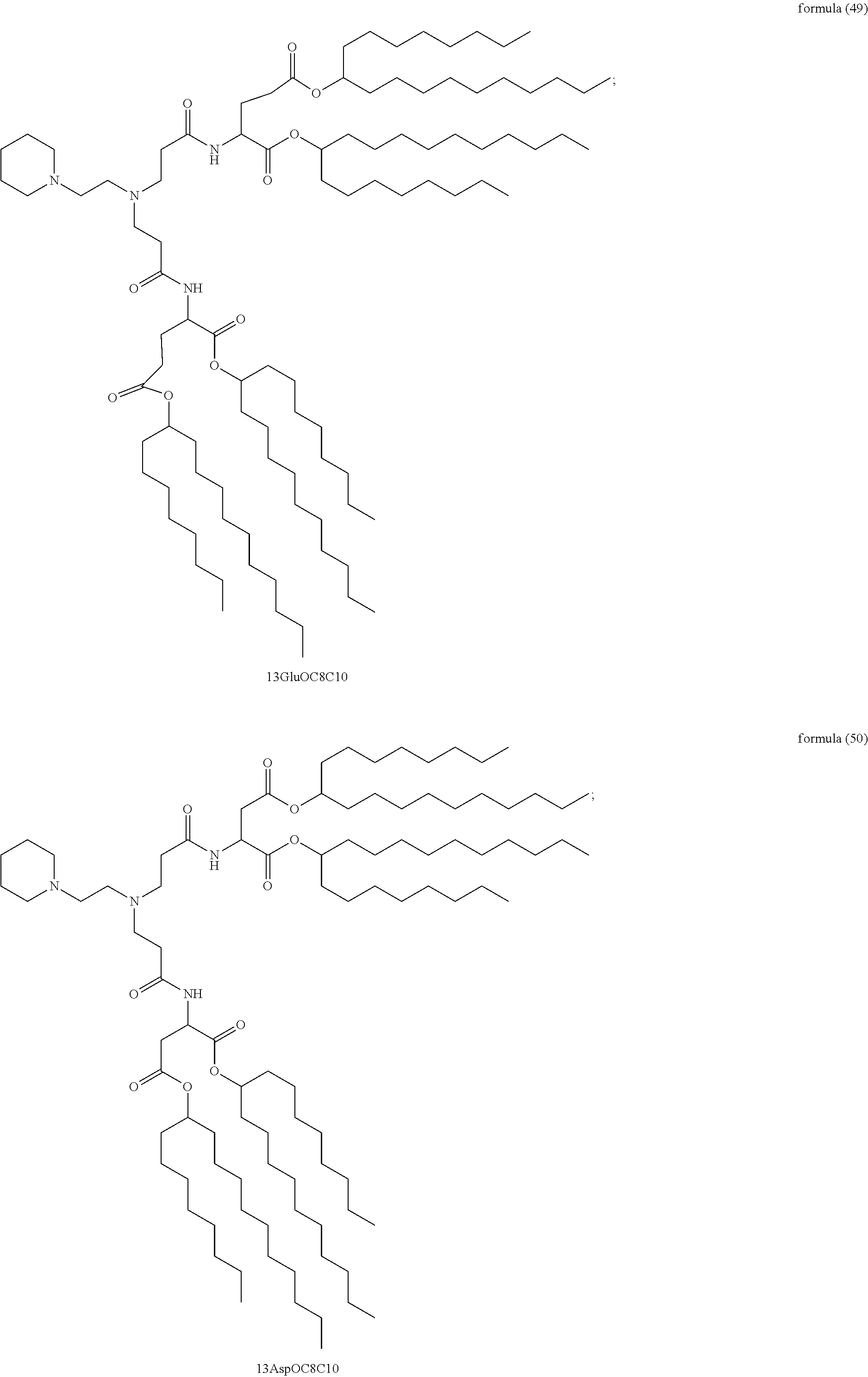 [SMILES: CCCCCCCCCCC(CCCCCCCC)OC(=O)CCC(NC(=O)CCN(CCC(=O)NC(CCC(=O)OC(CCCCCCCC)CCCCCCCCCC)C(=O)OC(CCCCCCCC)CCCCCCCCCC)CCN1CCCCC1)C(=O)OC(CCCCCCCC)CCCCCCCCCC ; CCCCCCCCCCC(CCCCCCCC)OC(=O)CC(NC(=O)CCN(CCC(=O)NC(CC(=O)OC(CCCCCCCC)CCCCCCCCCC)C(=O)OC(CCCCCCCC)CCCCCCCCCC)CCN1CCCCC1)C(=O)OC(CCCCCCCC)CCCCCCCCCC]

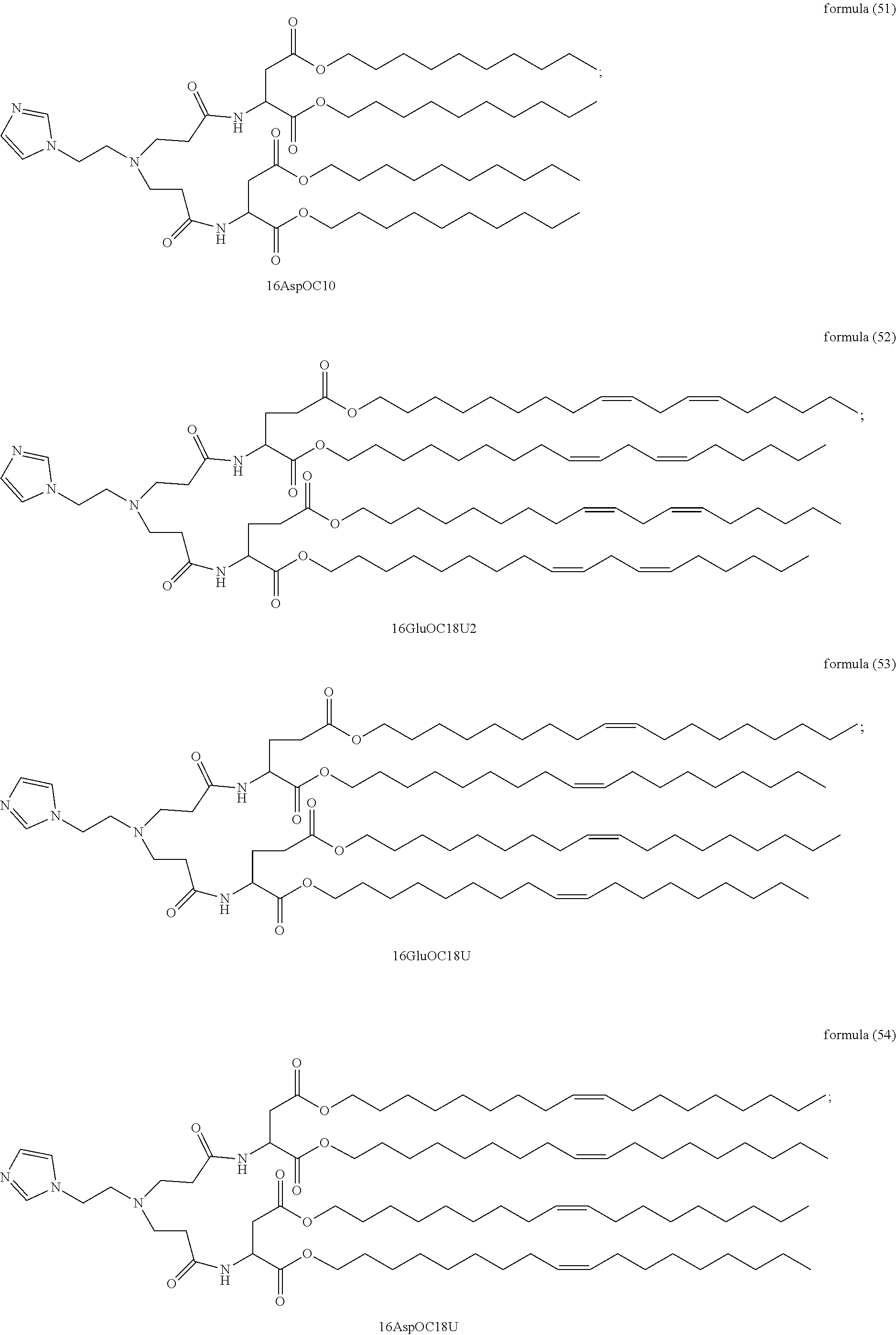

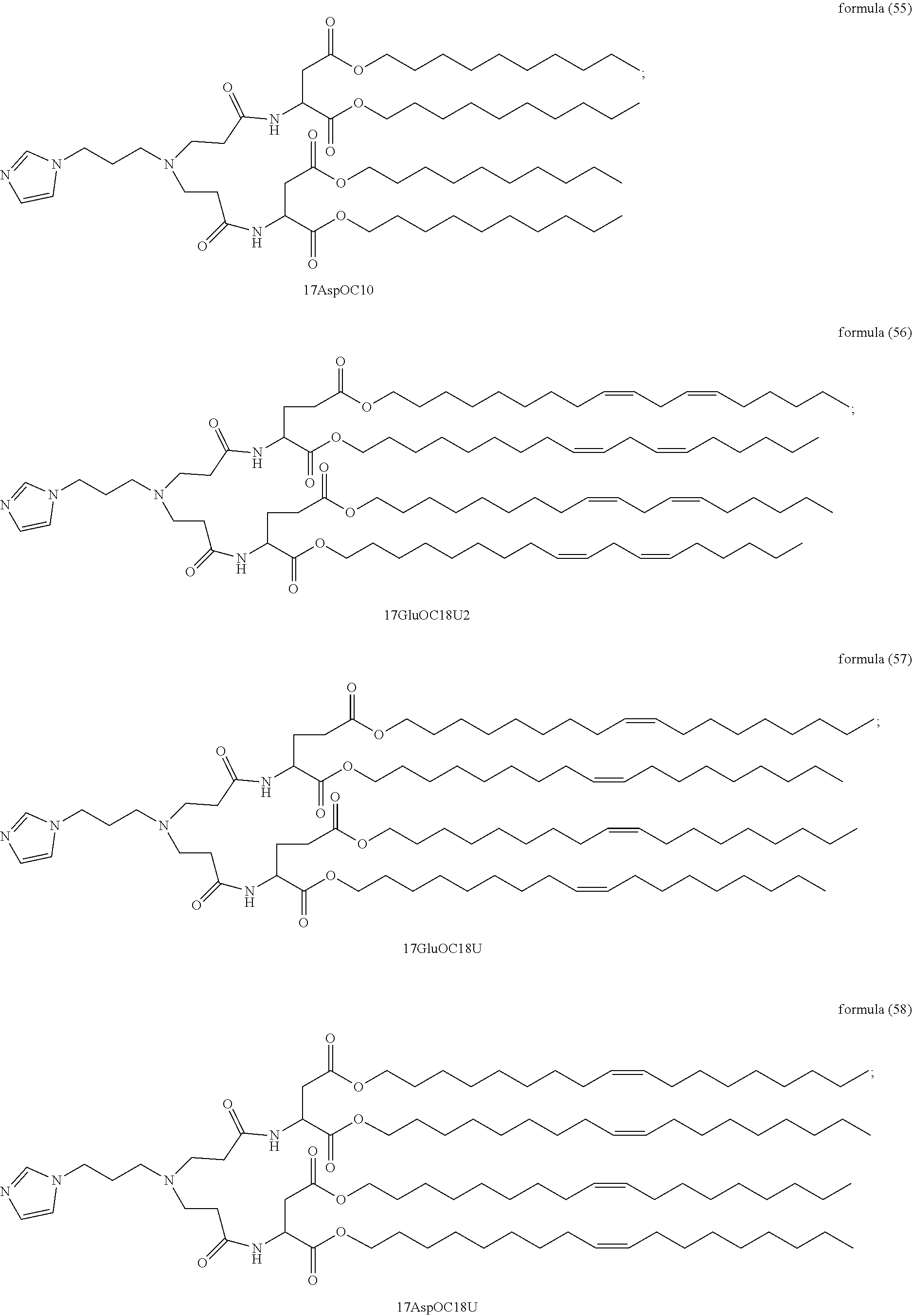

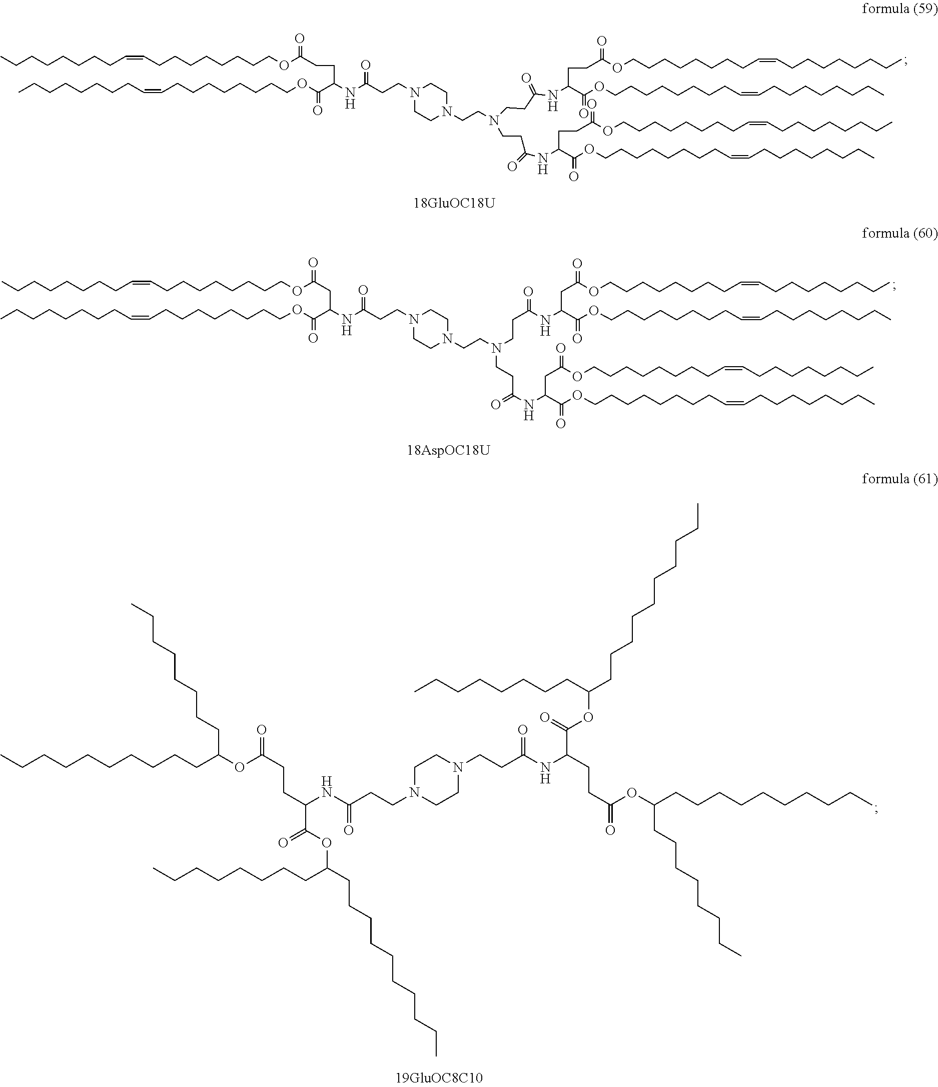

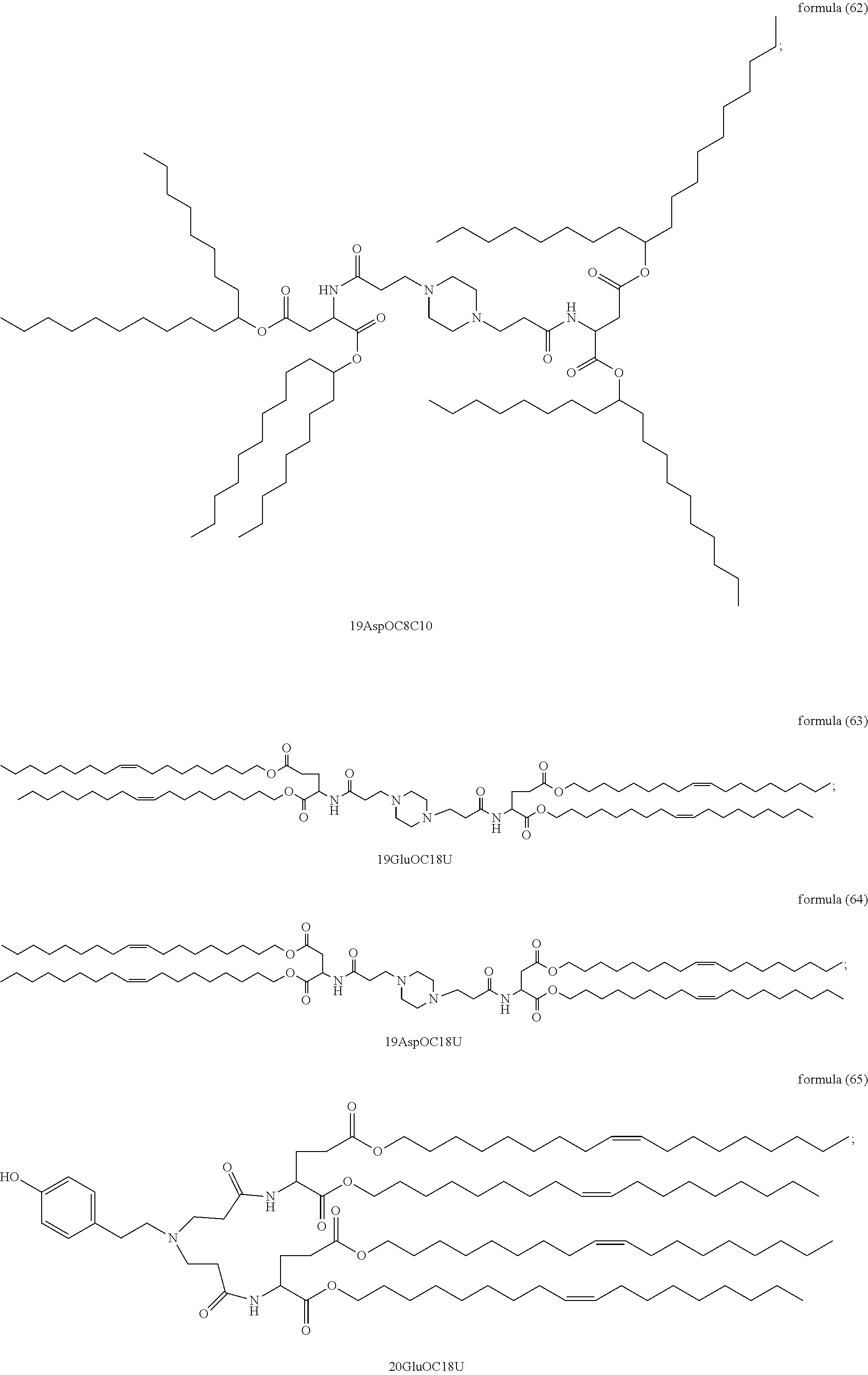

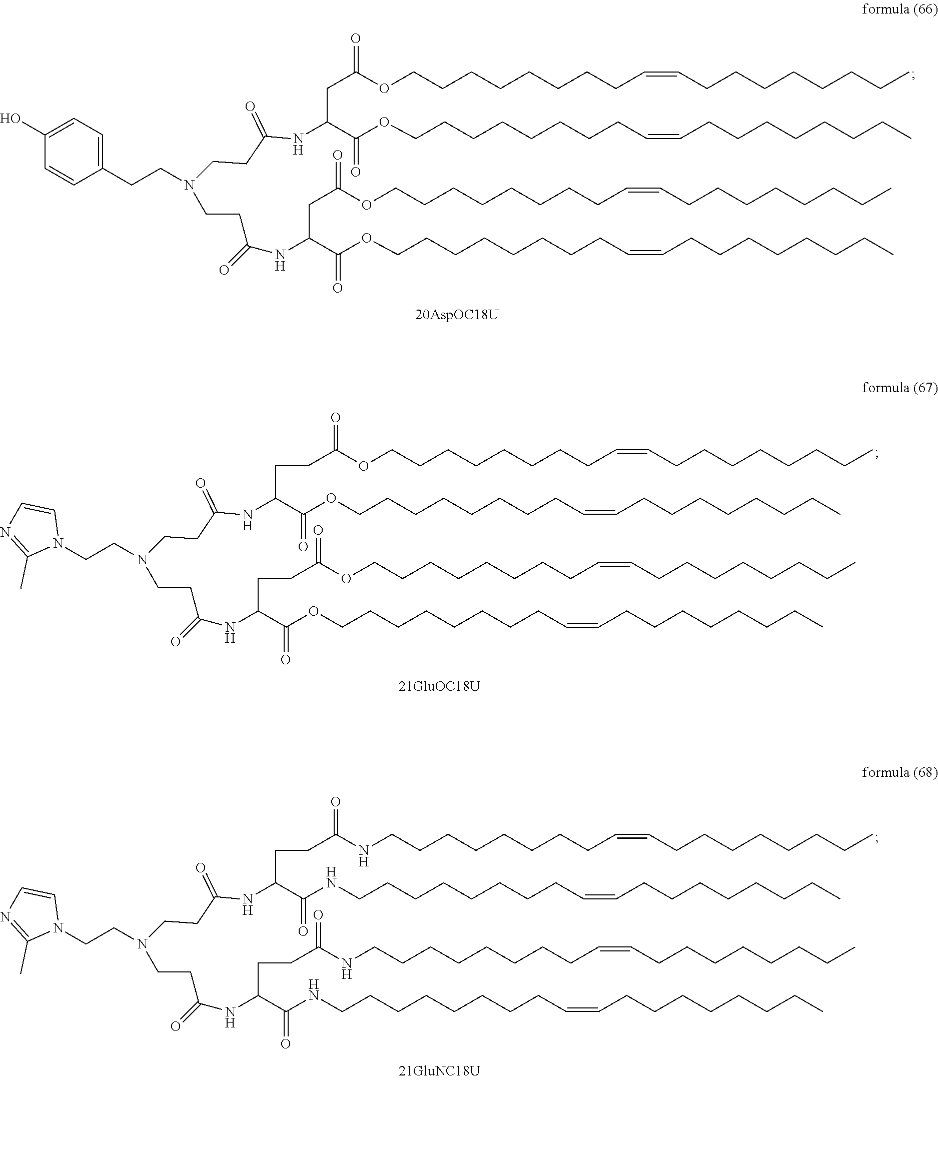 [SMILES: CCCCCCCC/C=C\CCCCCCCCOC(=O)CC(NC(=O)CCN(CCC(=O)NC(CC(=O)OCCCCCCCC/C=C\CCCCCCCC)C(=O)OCCCCCCCC/C=C\CCCCCCCC)CCc1ccc(O)cc1)C(=O)OCCCCCCCC/C=C\CCCCCCCC ; CCCCCCCC/C=C\CCCCCCCCOC(=O)CCC(NC(=O)CCN(CCC(=O)NC(CCC(=O)OCCCCCCCC/C=C\CCCCCCCC)C(=O)OCCCCCCCC/C=C\CCCCCCCC)CCn1ccnc1C)C(=O)OCCCCCCCC/C=C\CCCCCCCC ; CCCCCCCC/C=C\CCCCCCCCNC(=O)CCC(NC(=O)CCN(CCC(=O)NC(CCC(=O)NCCCCCCCC/C=C\CCCCCCCC)C(=O)NCCCCCCCC/C=C\CCCCCCCC)CCn1ccnc1C)C(=O)NCCCCCCCC/C=C\CCCCCCCC]

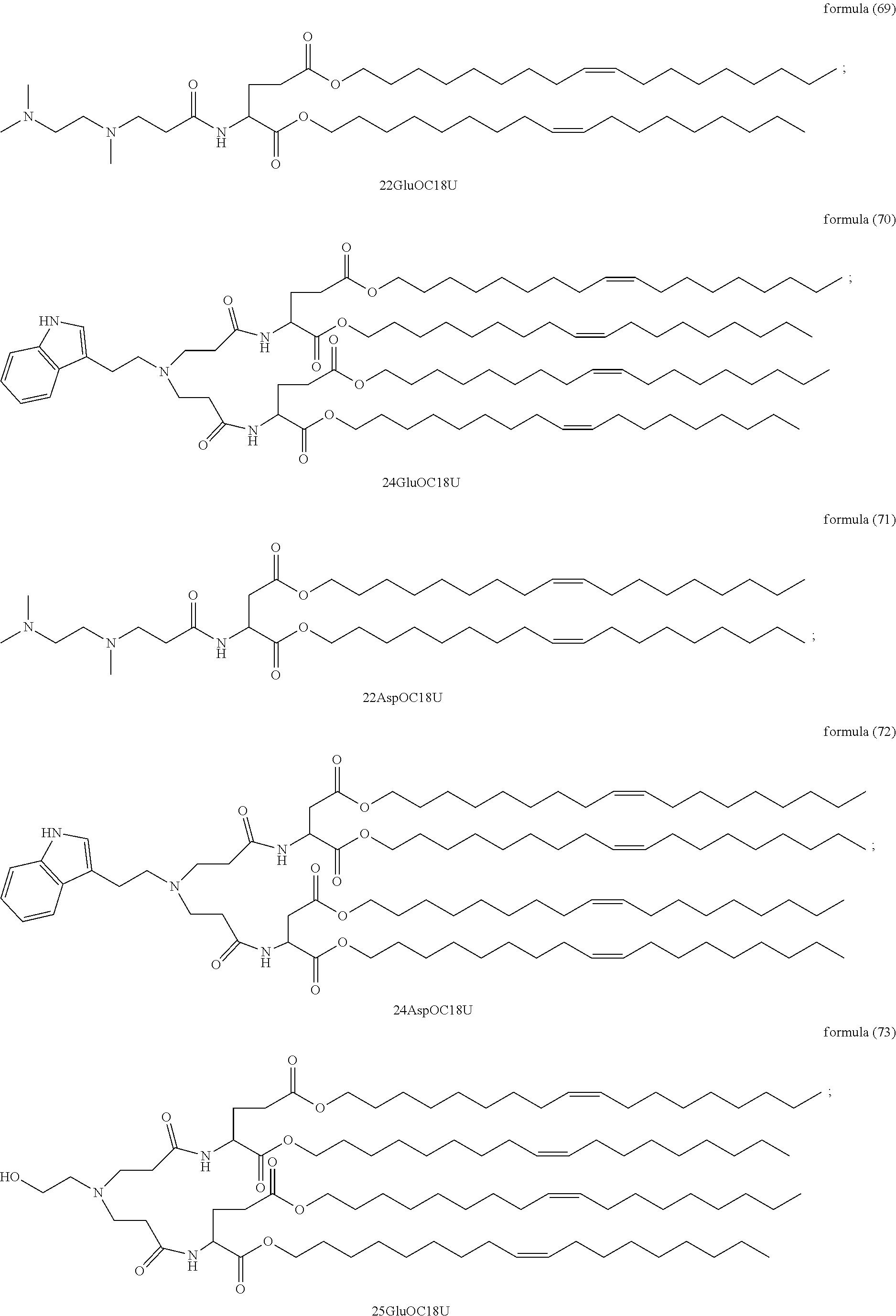

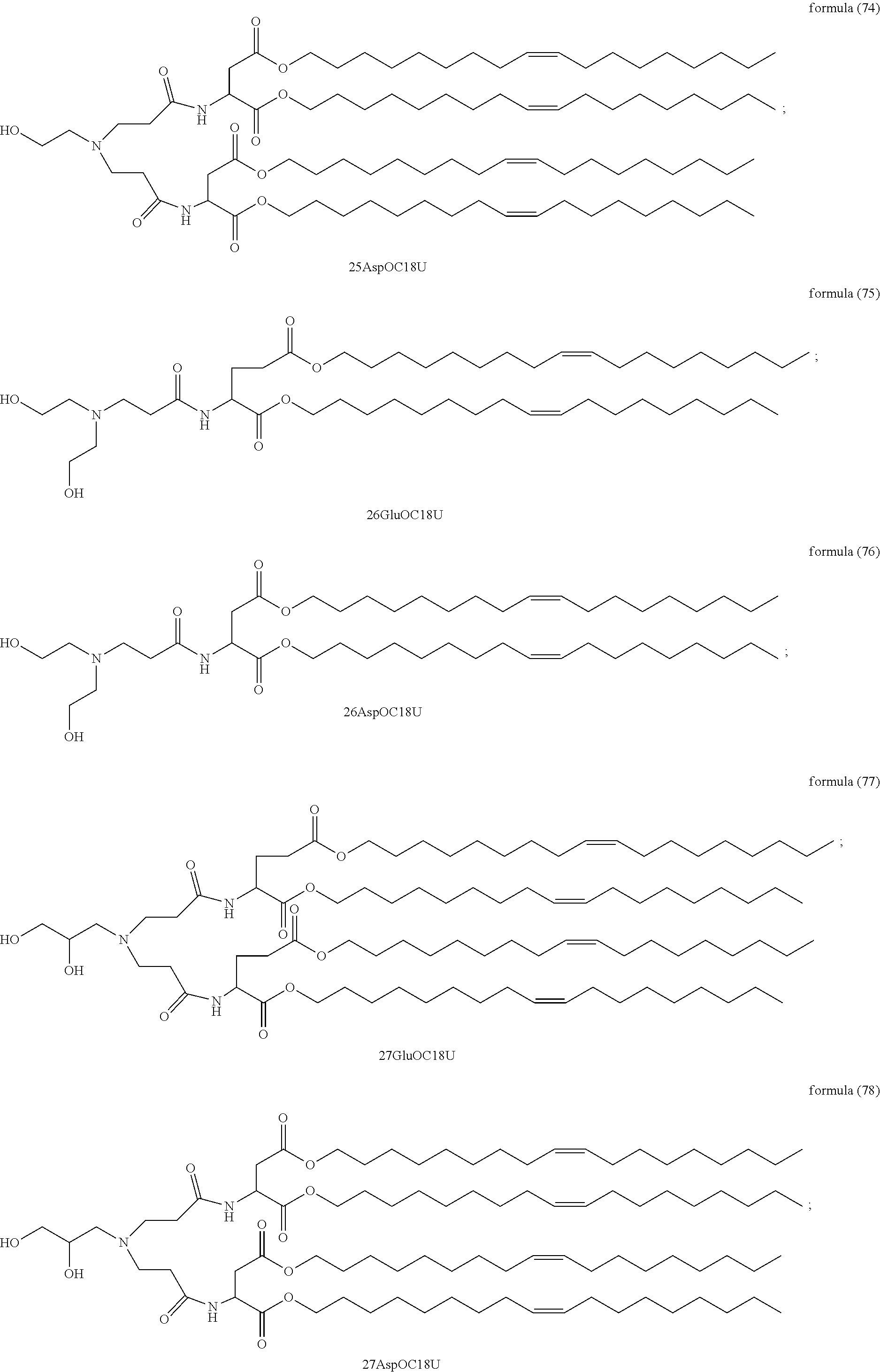

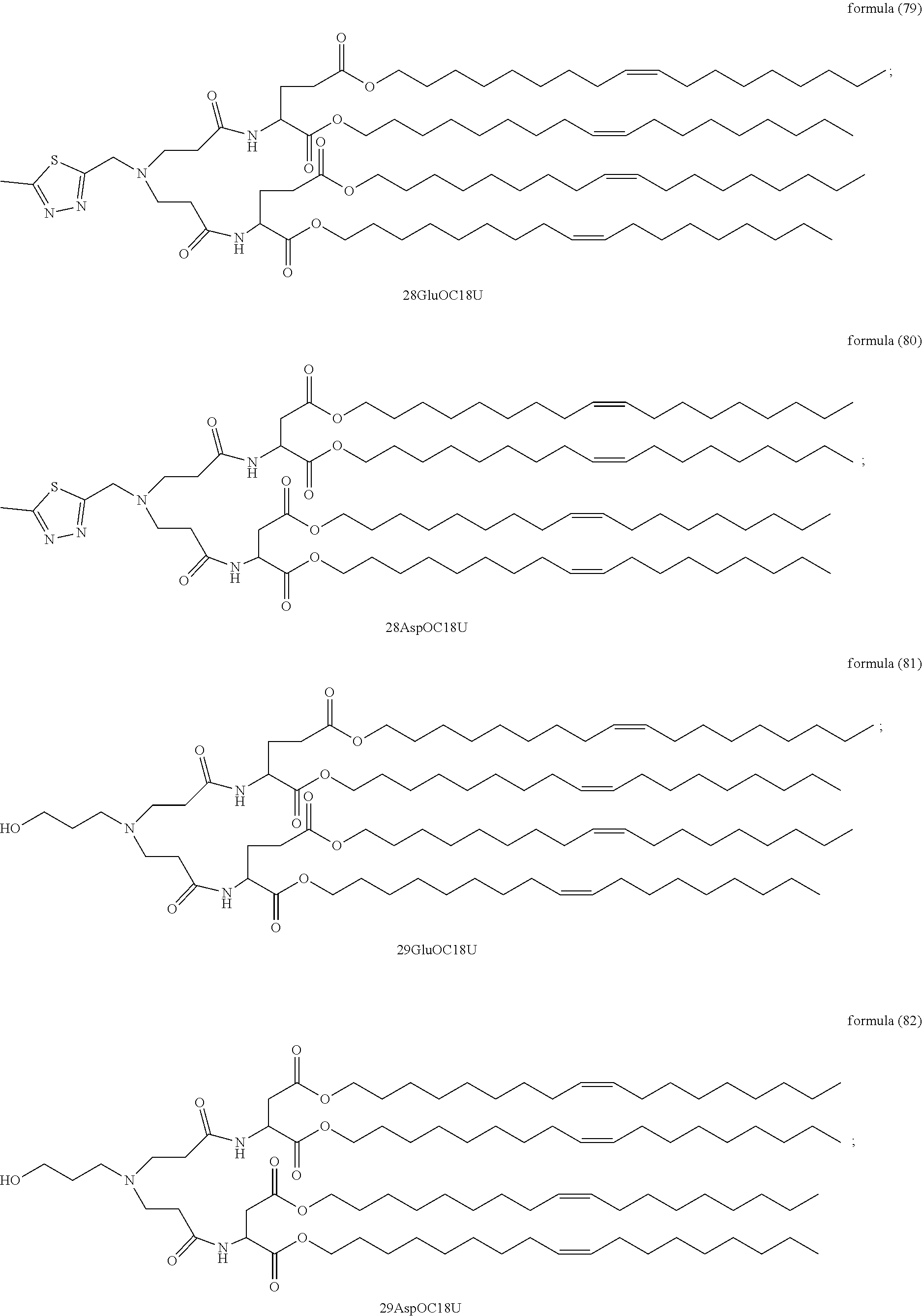

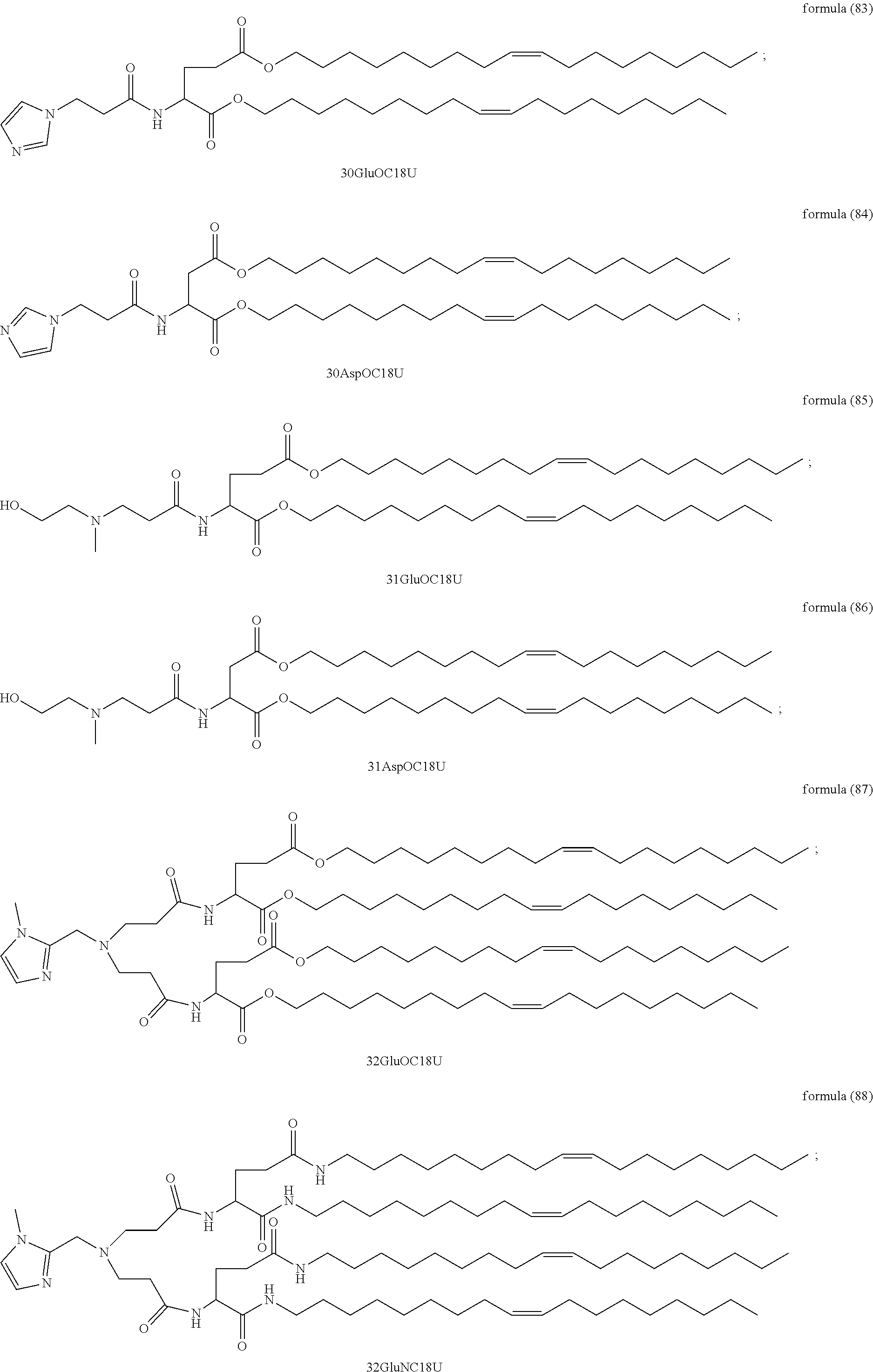

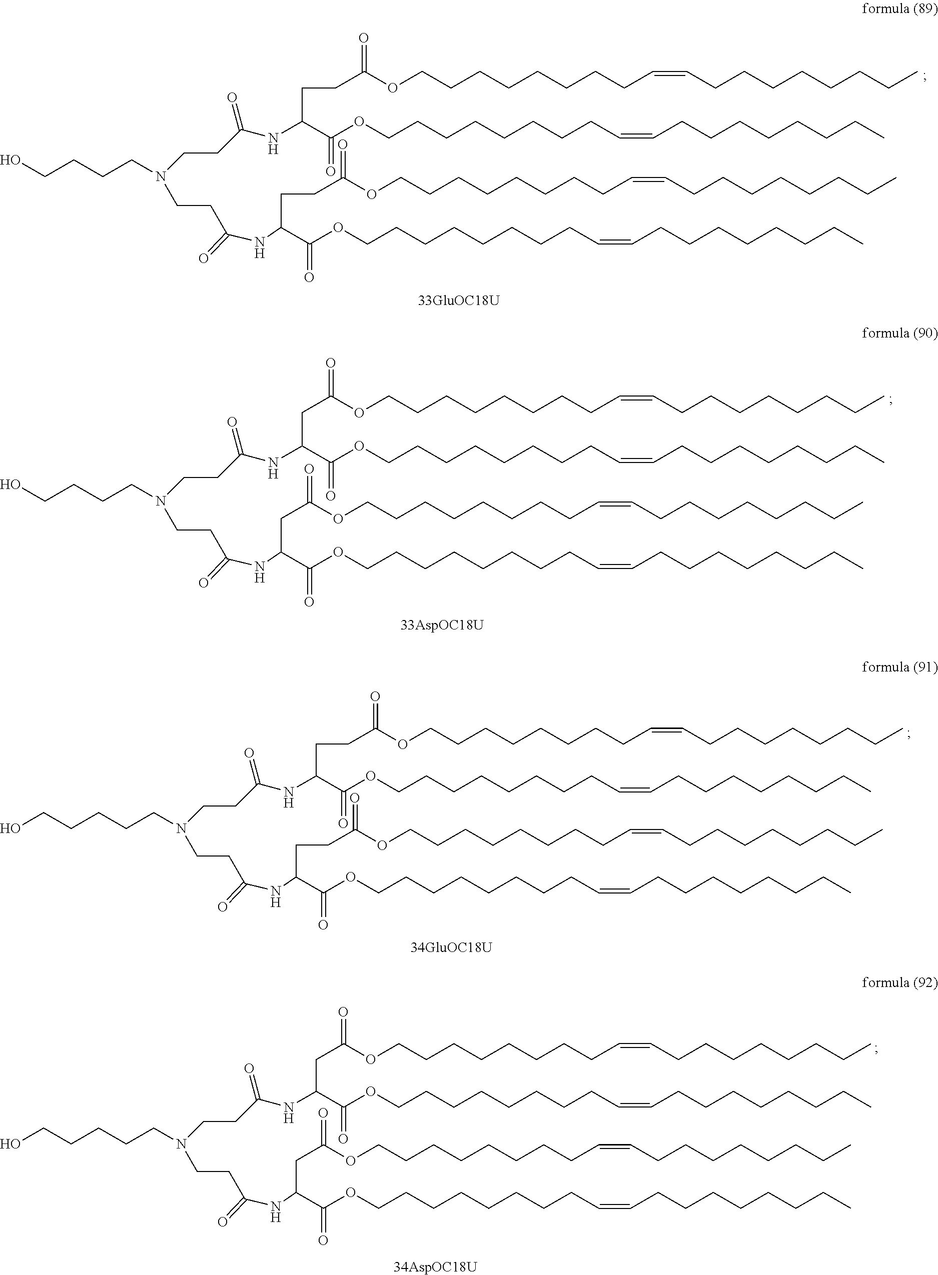

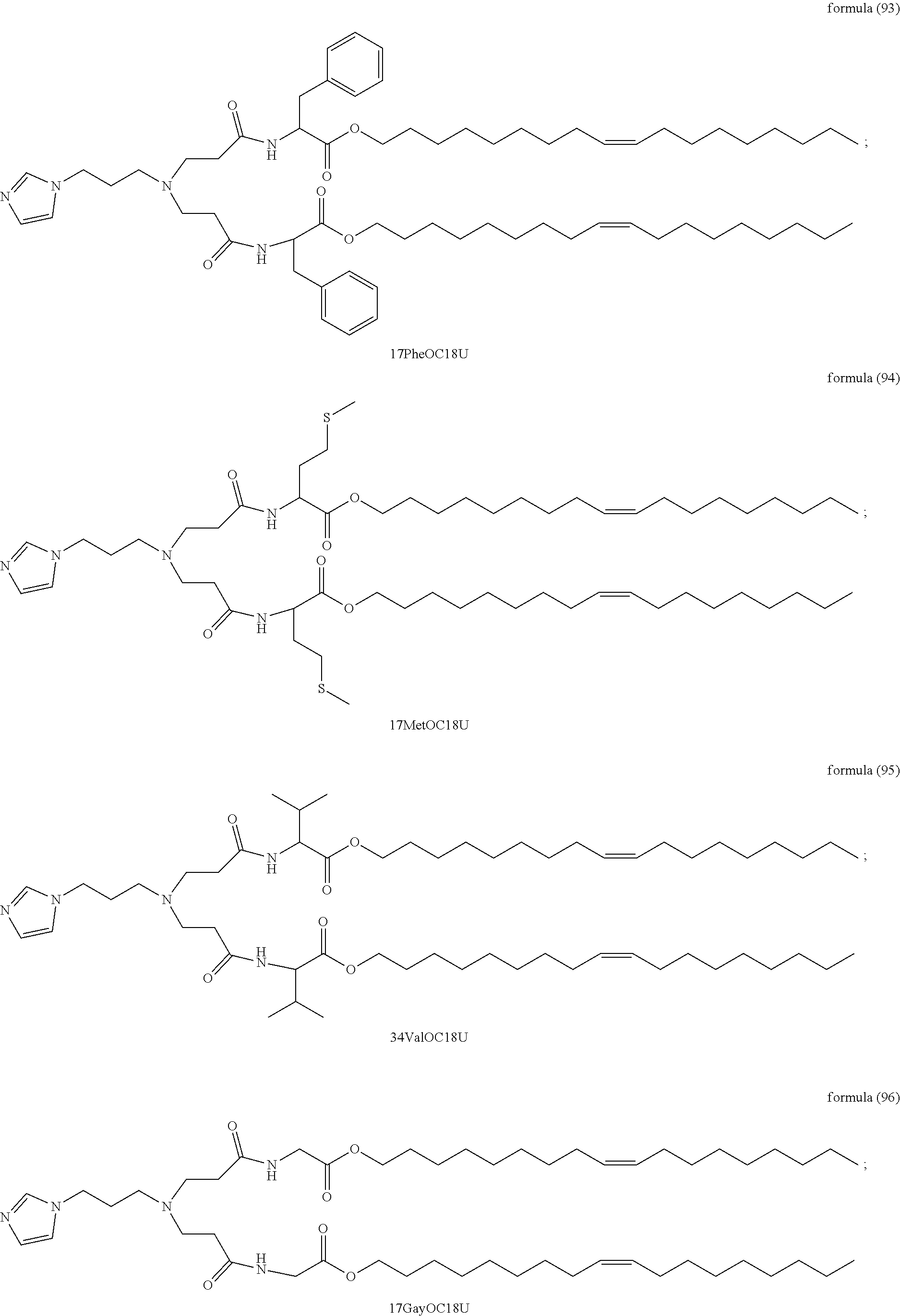

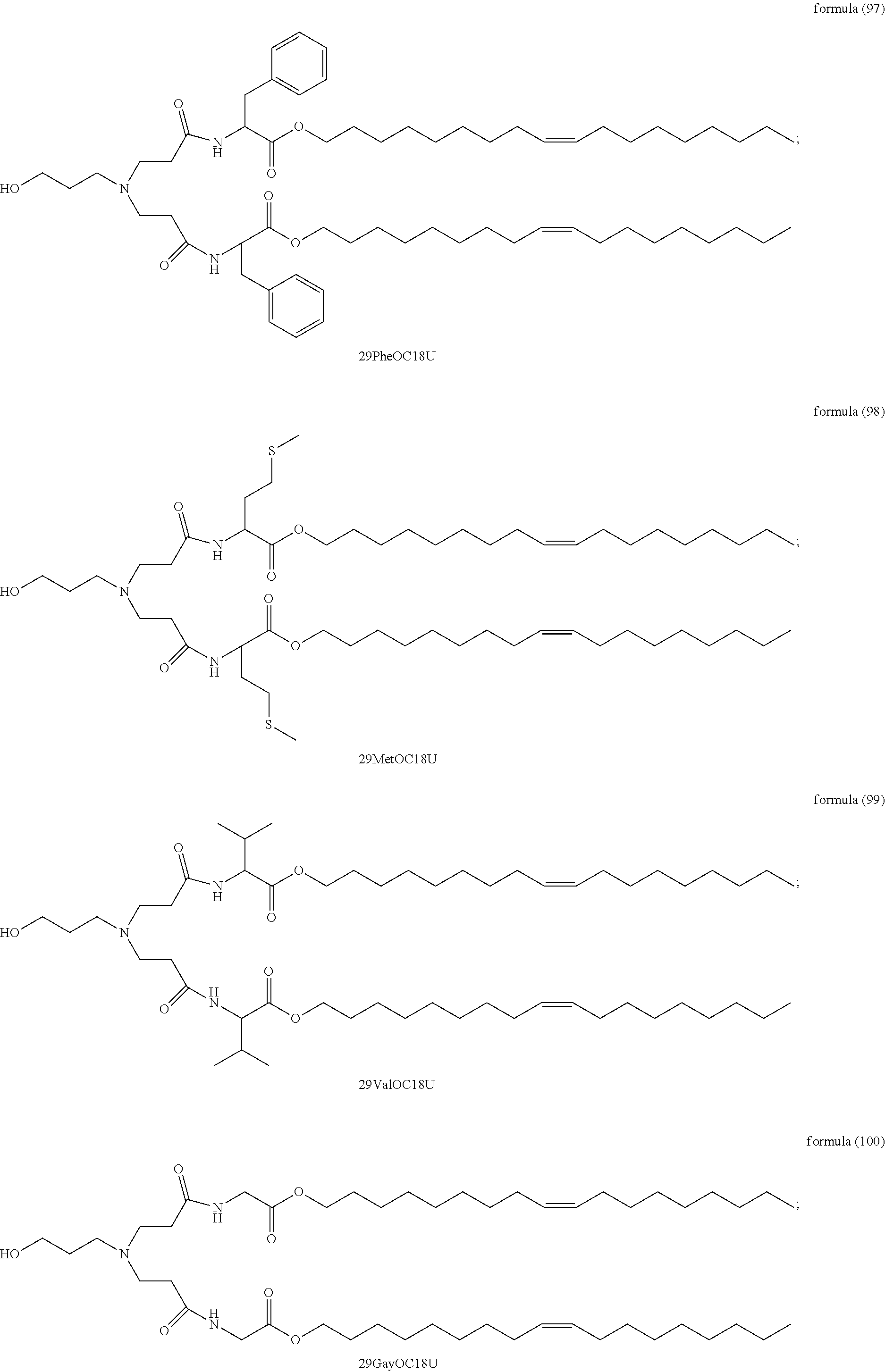 [SMILES: CCCCCCCC/C=C\CCCCCCCCOC(=O)C(Cc1ccccc1)NC(=O)CCN(CCCO)CCC(=O)NC(Cc1ccccc1)C(=O)OCCCCCCCC/C=C\CCCCCCCC ; CCCCCCCC/C=C\CCCCCCCCOC(=O)C(CCSC)NC(=O)CCN(CCCO)CCC(=O)NC(CCSC)C(=O)OCCCCCCCC/C=C\CCCCCCCC ; CCCCCCCC/C=C\CCCCCCCCOC(=O)C(NC(=O)CCN(CCCO)CCC(=O)NC(C(=O)OCCCCCCCC/C=C\CCCCCCCC)C(C)C)C(C)C ; CCCCCCCC/C=C\CCCCCCCCOC(=O)CNC(=O)CCN(CCCO)CCC(=O)NCC(=O)OCCCCCCCC/C=C\CCCCCCCC]

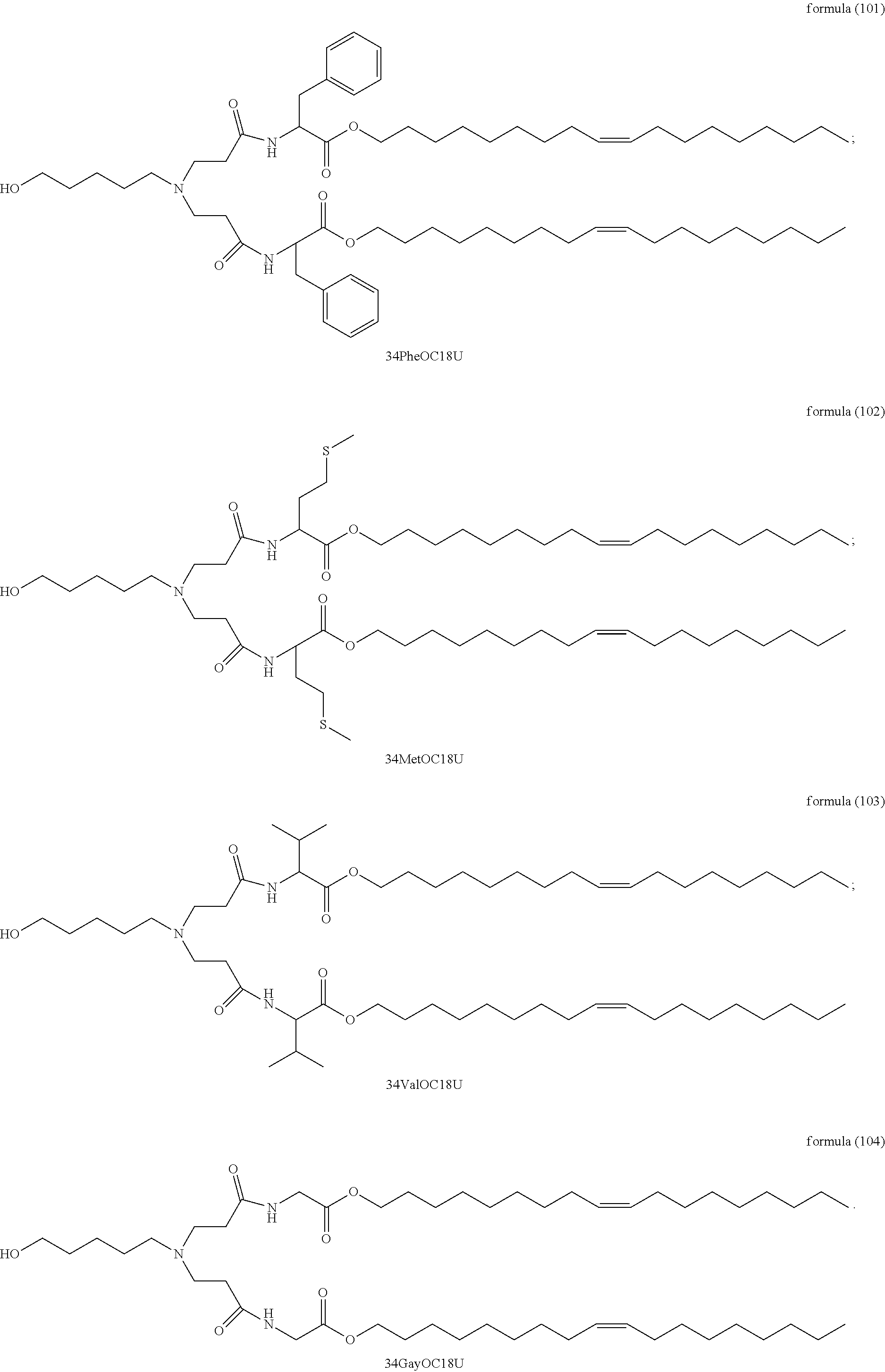 [SMILES: CCCCCCCC/C=C\CCCCCCCCOC(=O)C(Cc1ccccc1)NC(=O)CCN(CCCCCO)CCC(=O)NC(Cc1ccccc1)C(=O)OCCCCCCCC/C=C\CCCCCCCC ; CCCCCCCC/C=C\CCCCCCCCOC(=O)C(CCSC)NC(=O)CCN(CCCCCO)CCC(=O)NC(CCSC)C(=O)OCCCCCCCC/C=C\CCCCCCCC ; CCCCCCCC/C=C\CCCCCCCCOC(=O)C(NC(=O)CCN(CCCCCO)CCC(=O)NC(C(=O)OCCCCCCCC/C=C\CCCCCCCC)C(C)C)C(C)C ; CCCCCCCC/C=C\CCCCCCCCOC(=O)CNC(=O)CCN(CCCCCO)CCC(=O)NCC(=O)OCCCCCCCC/C=C\CCCCCCCC]

The second aspect of the present disclosure provides a preparation method of the amino acid backbone ionizable lipid according to the first aspect, including: tail compounds containing amino acid backbone or stereoisomers, tautomers, reacting with organic amine compounds through Michael addition reactions to produce the amino acid backbone ionizable lipid;
where a structural formula of tail compounds containing amino acid backbone is represented as formula (a):

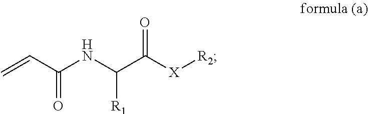

where R₁ is selected from

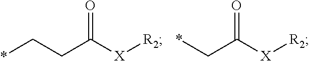

C2-C10 alkyl; C2-C10 heteroalkylene; arylalkyl or non-existence;
R₂ is selected from C3-C24 linear alkyl; C4-C24 linear alkenyl;

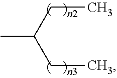

where n1=2-3, n2=3-24, n3=3-24;
X is O or NH.

In some embodiments, a reaction temperature for Michael addition reaction is 70-110° C.

In some embodiments, the reaction temperature for Michael addition reaction is 80-100° C.

In some embodiments, the reaction temperature for Michael addition reaction is 85-95° C.

In some embodiments, a reaction time for Michael addition reaction is 36-60 hours (h).

In some embodiments, the reaction time for Michael addition reaction is 40-56 h.

In some embodiments, the reaction time for Michael addition reaction is 44-52 h.

In some embodiments, the organic amine compound is selected from:

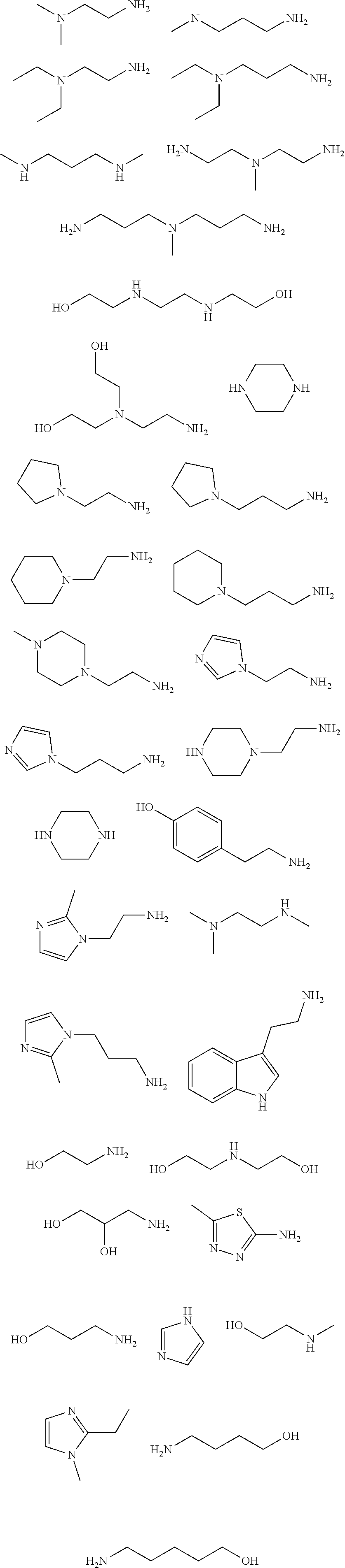

In some embodiments, a preparation method of tail compounds containing amino acid backbone including: preparing tail compounds containing amino acid backbone by esterification reaction
between acryloyl chloride and compound a1

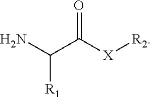

In some embodiments, a reaction temperature for esterification reaction is 15-40° C.

In some embodiments, the reaction temperature for esterification reaction is 20-35° C.

In some embodiments, a reaction time for esterification reaction is 12-24 hours (h).

In some embodiments, a reaction time for esterification reaction is 12-16 h.

In some embodiments, a reaction system of the esterification reaction includes organic base catalyst.

In some embodiments, the organic base catalyst is triethylamine.

In some embodiments, a molar ratio of acryloyl chloride to compound a1 is (1-2):1.

In some embodiments, a molar ratio of acryloyl chloride to compound a1 is (1-1.5):1.

In some embodiments, a preparation method of compound a1 including: reacting Boc amino acid with alkyl alcohols or vinyl alcohols, reaction product undergoes deprotection reaction with trifluoroacetic acid to obtain compound a1.

In some embodiments, reaction system for preparing compound a1 also includes an active agent and an acid promoter.

In some embodiments, the active agent includes 1-(3-dimethylaminopropyl)-3-ethylcarbodiimide hydrochloride (EDCI).

In some embodiments, the acid promoter includes 4-dimethylaminopyridine (DMAP).

In some embodiments, a reaction temperature for preparing compound a1 is 15-40° C.

In some embodiments, a reaction temperature for deprotection reaction is 15-40° C.

The third aspect of the present disclosure provides a lipid synthesis, including the amino acid backbone ionizable lipid, sterol, and PEGylated lipid described in the first aspect.

In some embodiments, the lipid synthesis also includes neutral auxiliary lipid.

In some embodiments, the neutral auxiliary lipid includes at least one of 1,2-distearoyl-sn-glycero-3-phosphocholine (DSPC), 1,2-dioleoyl-sn-glycero-3-phosphocholine (DOPC), Dioleoylphosphatidylethanolamine (DOPE), Dimyristoyl phosphatidylcholine (DMPC), and 1-palmitoyl-2-oleoyl-sn-glycero-3-phosphocholine (POPC).

In some embodiments, the PEGylated lipid includes at least one of PEG modified phosphatidylethanolamine, PEG modified phosphatidic acid, PEG modified ceramides, PEG modified dialkylamine, PEG modified diacylglycerol, or PEG modified dialkylglycerol.

In some embodiments, the sterol includes at least one of cholesterol and β-sitosterol.

In some embodiments, when the lipid synthesis includes cholesterol, auxiliary phospholipid, and PEGylated lipid, a molar ratio of amino acid backbone ionizable lipid: cholesterol:auxiliary phospholipid:PEGylated lipid is (30-50):(35-60):(5-20):(1-5).

The fourth aspect of the present disclosure provides an application method of the amino acid backbone ionizable lipid described in the first aspect, or the lipid synthesis described in the third aspect in preparing a biologically active substance delivery system, where the delivery system includes particles, nanoparticles, liposomes, lipid nanoparticles, or microbubbles.

The fifth aspect of the present disclosure provides a pharmaceutical synthesis, including the lipid synthesis described in the third aspect and active substance; where the active substance includes at least one of nucleic acid molecules, small molecule compounds, peptides, and proteins.

In some embodiments, the pharmaceutical synthesis includes: carrier and active substance, where the carrier includes the lipid synthesis described in the third aspect; the active substance is encapsulated within or bound to a carrier.

In some embodiments, a preparation method of the pharmaceutical synthesis, including:
mixing the amino acid backbone ionizable lipid, sterol, neutral auxiliary lipid, and PEGylated lipid in ethanol solvent to prepare a lipid mixture solution; mixing the active substance with an acidic buffer solution to obtain a first mixture, mixing the first mixture with the lipid mixture solution, incubated to obtain a drug carrier which is the drug synthesis;
or dissolving the amino acid backbone ionizable lipid, sterol, and neutral auxiliary lipid in chloroform to obtain a solvent, blow dry with nitrogen to evaporate the solvent, adding acidic buffer solution or neutral buffer solution and mixing to prepare lipid nanoparticles for later use; mixing cationic peptides with active substance to obtain a second mixture, then mixing the second mixture with the lipid nanoparticles, and adding PEGylated lipid to prepare drug carriers.

In some embodiments, the cationic peptides is protamine.

In some embodiments, the acidic buffer solution pH=3-7; where the acidic buffer solution is sodium acetate buffer or sodium citrate buffer.

In some embodiments, when an active ingredient of the pharmaceutical synthesis includes nucleic acid molecules, a nitrogen to phosphorus ratio of the amino acid backbone ionizable lipid and nucleic acid molecules is (1-100):1.

In some embodiments, when an active ingredient of the pharmaceutical synthesis includes nucleic acid molecules, a nitrogen to phosphorus ratio of the amino acid backbone ionizable lipid and nucleic acid molecules is (1-30):1.

In some embodiments, when an active ingredient of the pharmaceutical synthesis includes nucleic acid molecules, a nitrogen to phosphorus ratio of the amino acid backbone ionizable lipid and nucleic acid molecules is (1-10):1.

In some embodiments, the nucleic acid molecules includes at least one type of siRNA, miRNA, mRNA, circRNA, antisense RNA, CRISPR guide RNAs, replicable RNA, cyclic dinucleotides poly IC, CpG ODN, plasmid DNA and microcyclic DNA.

In some embodiments, the proteins includes at least one of cell colony-stimulating factors, interleukins, lymphotoxins, interferon proteins, tumor necrosis factor, antibodies, and protein antigens.

The sixth aspect of the present disclosure provides an application method of the amino acid backbone ionizable lipid described in the first aspect, or the pharmaceutical synthesis described in the fifth aspect, in preparing nucleic acid drugs, gene vaccines, peptide or protein drugs, and small molecule drugs.

Beneficial effects of the present disclosure are:
(1) The present disclosure proposes an amino acid backbone ionizable lipid, which is modified with amino acids as a core and has many ester groups and peptide bonds. After effectively releasing RNA in vivo, it can be quickly hydrolyzed by enzymes, easily metabolized and cleared in vivo, has biodegradability. The amino acid backbone ionizable lipid has four tail structures, which can increase cross-sectional area of lipid tails, helping RNA and other drugs escape from endosomes, and enhancing transfection efficiency. Charge of the ionizable lipid can change with the pH of the environment, becoming electrically neutral under physiological conditions, reducing cytotoxicity caused by excessive positive charges, thereby increasing stability of lipid nanoparticles, and helping to prolong a circulation time of loaded nucleic acid drugs and improving pharmacokinetic characteristics.
(2) The LNP made from ionizable lipid provided by the present disclosure, auxiliary phospholipid, cholesterol, and PEG lipid has superior nucleic acid carrier performance and can effectively deliver nucleic acid drugs such as siRNA, mRNA, pDNA into cells to exert effects.
(3) The preparation method of the amino acid skeleton ionized lipid of the present disclosure is different from rigorous and complex synthesis route of traditional cationic lipids. Through Michael addition can obtain an ionizable lipid library, which has advantages of simple synthesis route, clear reaction mechanism, and easy high-throughput screening.

### BRIEF DESCRIPTION OF THE DRAWINGS

FIG. 1 shows relative luciferase activity of cells transfected with different lipid nanoparticles;

FIG. 2 (a) shows relative luciferase activity results of lipid nanoparticles with different neutral phospholipids for cell transfection; FIG. 2 (b) shows relative luciferase activity results of cell transfection with lipid nanoparticles of different component ratios;

FIG. 3 (c) shows relative luciferase activity results of lipid nanoparticles with different nitrogen phosphorus ratios for cell transfection; FIG. 3 (d) shows relative luciferase activity of lipid nanoparticles transfected into cells using different buffer solutions;

FIG. 4 shows fluorescence microscopy of cell transfection with different lipid nanoparticles in Jurkat T cell line;

FIG. 5 shows imaging images of different lipid nanoparticles transfected into mice in vivo;

FIG. 6 shows relative luciferase activity of different lipid nanoparticles transfected into mice in vivo;

FIG. 7 shows relative luciferase activity of different lipid nanoparticles transfected into spleen and liver of mice in vivo;

FIG. 8 shows relative luciferase activity ratio of spleen and liver transfected with different lipid nanoparticles in mice.

### DETAILED DESCRIPTION

The following provides further detailed explanations of a content of the present disclosure through specific embodiments. The raw materials used in the following examples, unless otherwise specified, can be obtained from conventional commercial channels or prepared and separated through simple synthesis. The processes used, unless otherwise specified, are all conventional processes in this field.

The specific steps of the preparation method for the amino acid backbone ionizable lipid of the present disclosure are:

### s1: Synthesizing Amino Acid Backbone Hydrophobic Tail

specific steps are: adding 10 mmol Boc amino acid, 150 mL dichloromethane (DCM) and magnetite, 22 mmol 1-(3-dimethylaminopropyl)-3-ethylcarbodiimide hydrochloride (EDCI), 20 mmol 4-dimethylaminopyridine (DMAP) in a 250 mL reaction flask, stirring at room temperature for 15 minutes, then adding 20 mmol alkyl alcohol and react for 24 hours until the reaction is complete to obtain reaction mixture. Transferring the reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent to obtain product A. The product A is separated by thin-layer chromatography column.

Removing amino protecting group: dissolving the product in 30 mL DCM, adding 20 mL trifluoroacetic acid (TFA) and stirring at room temperature for 4 h. Using a vacuum rotary evaporator to remove organic solvents from reaction solution, then dissolving again in 150 mL DCM, washing with saturated sodium bicarbonate (2×100 mL), and extracted with saturated saline solution (2×100 mL). Collecting organic layer, drying with anhydrous magnesium sulfate and filtering, then using a vacuum rotary evaporator to remove organic solvent to obtain product B. The product B obtained does not require further purification and can proceed to next reaction step.

### s2: Synthesizing Connecting Group

adding 5 mmol of the amino acid backbone hydrophobic tail synthesized in s1, 7.5 mmol Triethylamine (TEA), and 20 mL DCM in a three necked flask containing magnetite. The three necked flask is pre-cooled in an ice bath for 30 minutes; slowly adding dropwise 6.25 mmol of acryloyl chloride (pre-mixed in 10 mL of dichloromethane) dropwise using a constant pressure funnel. After the acryloyl chloride is added dropwise, removing the ice bath. The reaction is left overnight at room temperature, then using DCM (30 mL) to dilute and washing with 1M HCl (50 mL). Organic layer is dried over anhydrous magnesium sulfate and filtered to obtain a product C, and the product C is separated by rapid chromatography column.

### s3: Reaction Between Head Group and Tail Group

selecting the alkyl tail synthesized in chemical equivalent s2, 100 mg of amine, and sequentially adding to a 3 mL reaction flask with tetrafluoroethylene lining. Heating the reaction flask at 90° C. for 48 hours. After the reaction is complete, product can be directly subjected to cell transfection experiments or separated by rapid chromatography column.

The Boc amino acid synthesized in the present disclosure using L-type or D-type amino acids to produce ionizable lipid with amino acid backbone containing L-type or D-type amino acid groups.

### Embodiment 1

Embodiment 1 provides an amino acid backbone ionizable lipid, and a preparation method is:

### s1: Synthesizing Amino Acid Backbone Hydrophobic Tail

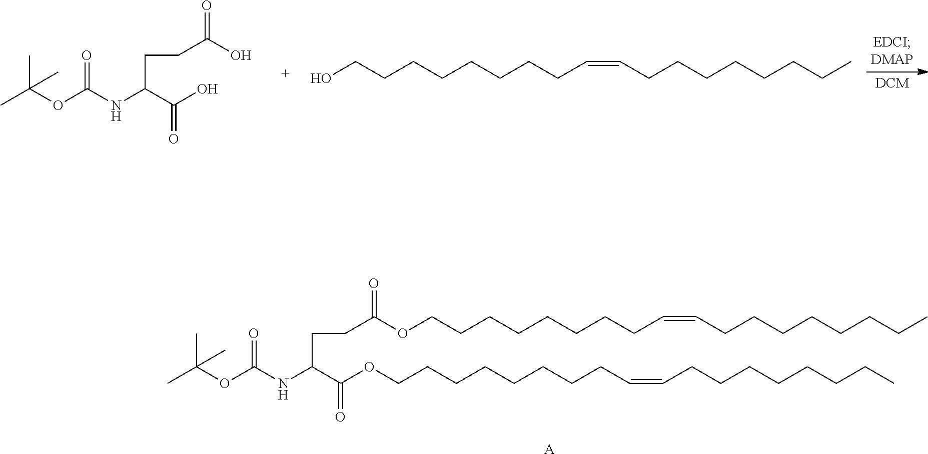

adding 10 mmol Boc amino acid, 150 mL dichloromethane (DCM) and magnetite, 22 mmol 1-(3-dimethylaminopropyl)-3-ethylcarbodiimide hydrochloride (EDCI), 20 mmol 4-dimethylaminopyridine (DMAP) in a 250 mL reaction flask, stirring at room temperature for 15 minutes, then adding 20 mmol oleyl alcohol and react for 24 hours. Using a thin layer chromatography (TLC) to detect progress of the reaction. When the reaction is complete, transferring reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent. Product is separated by a thin-layer chromatography column (petroleum ether:ethyl acetate=20:1) to obtain target product A with a yield of 73%.

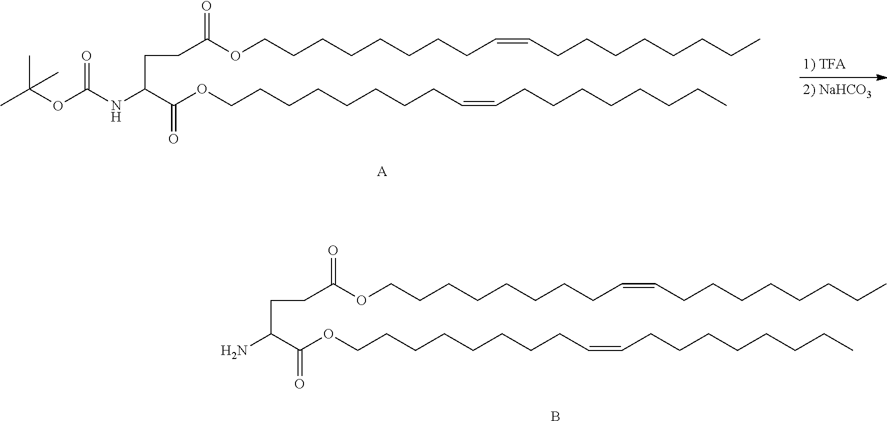

Removing amino protecting group: dissolving the target product A in 30 mL DCM, adding 20 mL TFA and stirring at room temperature for 4 h. Using a vacuum rotary evaporator to remove organic solvents from reaction solution, then dissolving again in 150 mL DCM, washing with saturated sodium bicarbonate (2×100 mL), and extracted with saturated saline solution (2×100 mL). Collecting organic layer, drying with anhydrous magnesium sulfate and filtering, then using a vacuum rotary evaporator to remove organic solvent to obtain product B. The product B does not require further purification and can proceed to next reaction step.

### s2: Synthesizing Connecting Group

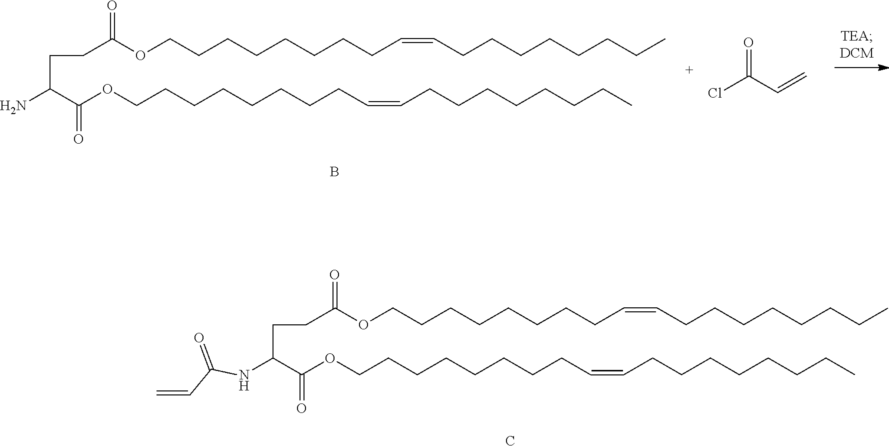

adding 5 mmol of the product B amino acid backbone hydrophobic tail, 7.5 mmol TEA, and 20 mL DCM in a three necked flask containing magnetite. The three necked flask is pre-cooled in an ice bath for 30 minutes; slowly adding dropwise 6.25 mmol of acryloyl chloride (pre-mixed in 10 mL of dichloromethane) dropwise using a constant pressure funnel. After the acryloyl chloride is added dropwise, removing the ice bath. The reaction is left overnight at room temperature, using TLC to detect reaction progress. Wait until the reaction is complete, using DCM (30 mL) to dilute and washing with 1M HCl (50 mL). Collecting organic layer, drying over anhydrous magnesium sulfate and filtering, then using a vacuum rotary evaporator to remove organic solvent. Product is separated by a thin-layer chromatography column (petroleum ether:ethyl acetate=5:1) to obtain target product C with a yield of 95%.

### Hydrogen Spectrum Data of the Obtained Product is:

¹H NMR (400 MHz, CDCl3): 6.37 (d, J=3.6, 1H), 6.30 (d, J=16 Hz, 1H), 5.79 (m, 1H), 5.6 (d, J=10.4 Hz, 1H), 5.38-5.29 (m, 4H), 4.72-4.67 (m, 1H), 4.15-4.02 (dt, J=6.8 Hz, 4H), 2.43-2.32 (m, 2H), 2.27-2.19 (m, 1H), 2.08-1.95 (m, 7H), 1.29-1.25 (m, 42H), 1.55-1.36 (m, 8H), 0.87 (t, J=13.6 Hz, 6H).

### s3: Reaction Between Head Group and Tail Group

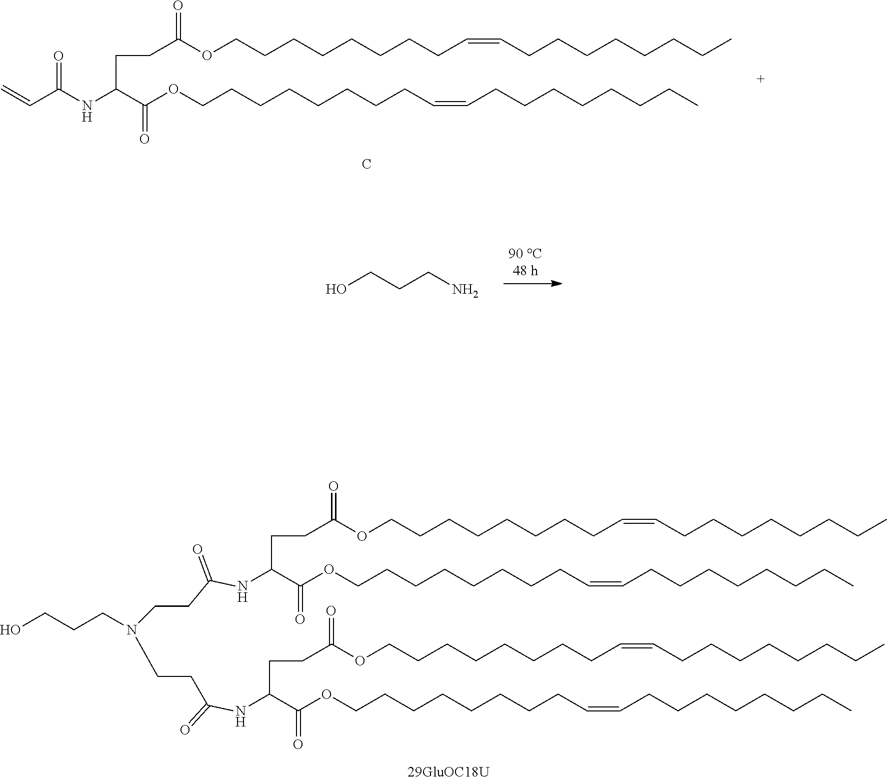

adding 100 mg of 3-aminopropanol and 2 times the chemical equivalent of target product C into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 29GluOC18U.

### Hydrogen Spectrum Data of the Obtained Product is:

¹H NMR (400 MHz, CDCl3): 4.78-4.65 (m, 4H), 4.08-4.03 (m, 4H), 3.19-3.02 (m, 8H), 2.81-2.77 (m, 2H), 2.63-2.42 (m, 10H), 1.80-1.48 (m, 20H), 1.39-1.25 (m, 20H), 0.87 (dd, J₁=6.8 Hz, J₂=5.2 Hz, 12H).

### Embodiment 2

Embodiment 2 provides an amino acid backbone ionizable lipid, and a preparation method is:

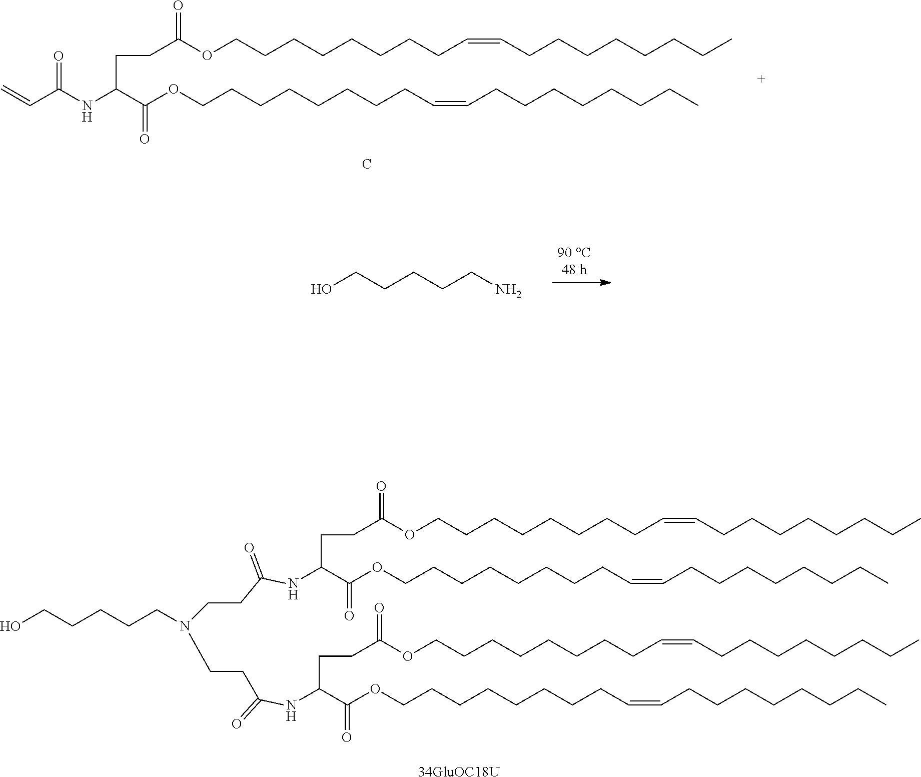

adding 100 mg of 5-aminopentanol and 2 times the chemical equivalent of target product C into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 34GluOC18U.

### Hydrogen Spectrum Data of the Obtained Product is:

¹H NMR (400 MHz, CDCl3): 7.73-7.38 (m, 2H), 5.41-5.35 (m, 8H), 4.58-4.53 (m, 2H), 4.14-4.06 (m, 8H), 3.69-3.66 (m, 2H), 3.16-3.10 (m, 8H), 2.81-2.77 (m, 2H), 2.47-2.41 (m, 4H), 2.24-1.98 (m, 20H), 1.67-1.59 (m, 10H), 1.45-1.41 (m, 4H), 1.36-1.27 (m, 88H), 0.91-0.88 (m, 12H).

### Embodiment 3

Embodiment 3 provides an amino acid backbone ionizable lipid, and a preparation method is:

### s1: Synthesizing Connecting Group

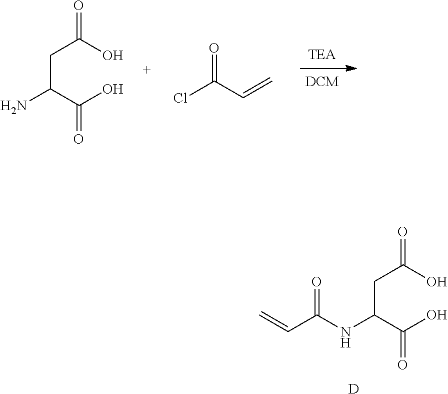

adding 10 mmol of L-aspartic acid, 30 mmol TEA, and 120 mL DCM in a three necked flask containing magnetite. The three necked flask is pre-cooled in an ice bath for 30 minutes; slowly adding dropwise 11 mmol of acryloyl chloride (pre-mixed in 20 mL of dichloromethane) dropwise using a constant pressure funnel. After the acryloyl chloride is added dropwise, removing the ice bath. The reaction is left overnight at room temperature, using TLC to detect reaction progress. Wait until the reaction is complete, using DCM (50 mL) to dilute and washing with 1M HCl (2×100 mL). Collecting organic layer, drying over anhydrous magnesium sulfate and filtering, then using a vacuum rotary evaporator to remove organic solvent. Product D can be used directly without purification.

### s2: Synthesizing Amino Acid Backbone Hydrophobic Tail

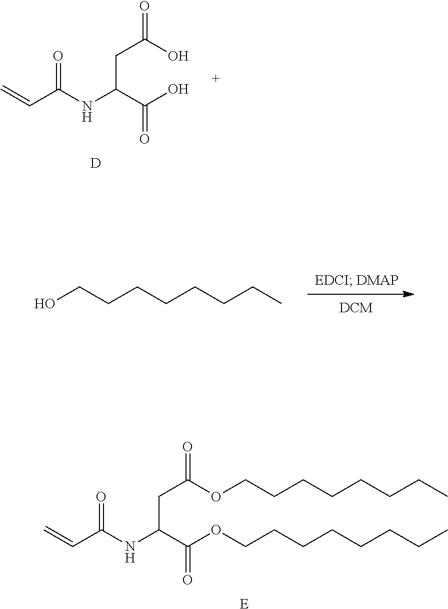

adding 5 mmol product D, 50 mL DCM and magnetite, 11 mmol EDCI, 5 mmol DMAP in a 100 mL reaction flask, stirring at room temperature for 15 minutes, then adding 11 mmol 1-octanol and react for 24 hours. Using TLC to detect progress of the reaction. When the reaction is complete, transferring reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent. Product is separated by a thin-layer chromatography column (petroleum ether:ethyl acetate=20:1) to obtain target product E with a yield of 43%.

### Hydrogen Spectrum Data of the Obtained Product is:

¹H NMR (400 MHz, CDCl3): 6.69 (d, J=8 Hz, 1H), 6.32 (d, J=8 Hz, 1H), 6.2-6.13 (m, 1H), 5.7 (d, J=10.4 Hz, 1H), 4.93-4.89 (m, 1H), 4.19-4.06 (m, 4H), 3.10-2.88 (m, 2H), 1.65-1.59 (m, 4H), 1.34-1.21 (m, 20H), 0.89 (t, J=6 Hz, 6H).

### s3: Reaction Between Head Group and Tail Group

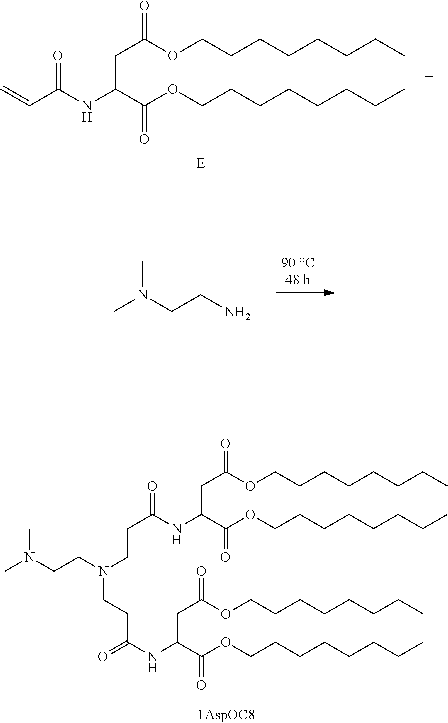

adding 100 mg of N,N-dimethylethylenediamine and 2 times the chemical equivalent of target product E into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 1AspOC8.

### Hydrogen Spectrum Data of the Obtained Product is:

¹H NMR (400 MHz, CDCl3): 4.78-4.65 (m, 4H), 4.08-4.03 (m, 4H), 3.19-3.02 (m, 8H), 2.81-2.77 (m, 2H), 2.63-2.42 (m, 10H), 1.80-1.48 (m, 20H), 1.39-1.25 (m, 20H), 0.87 (dd, J₁=6.8 Hz, J₂=5.2 Hz, 12H).

### Embodiment 4

Embodiment 4 provides an amino acid backbone ionizable lipid, and a preparation method is:

### s1: Synthesizing Amino Acid Backbone Hydrophobic Tail

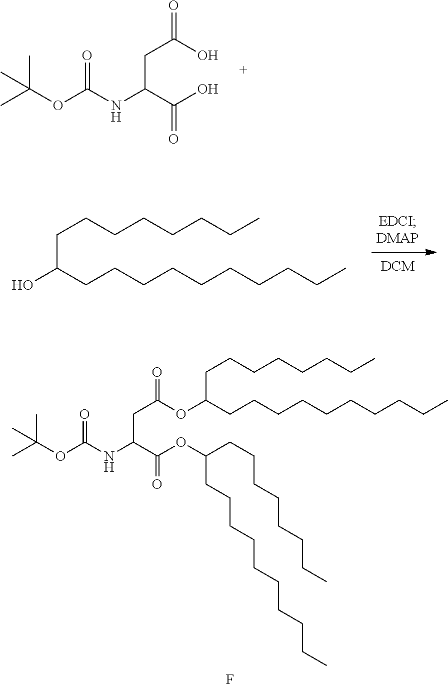

adding 10 mmol BOC-L aspartic acid, 150 mL DCM and magnetite, 22 mmol EDCI, 20 mmol DMAP in a 250 mL reaction flask, stirring at room temperature for 15 minutes, then adding 20 mmol 9-nonadecanol and react for 24 hours. Using TLC to detect progress of the reaction. When the reaction is complete, transferring reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent. Product is separated by a thin-layer chromatography column (petroleum ether:ethyl acetate=30:1) to obtain target product F with a yield of 68%.

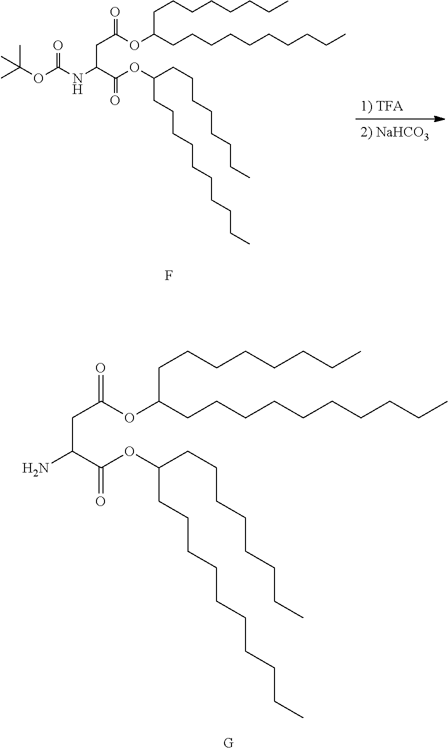

Removing amino protecting group: dissolving the target product F in 30 mL DCM, adding 20 mL TFA and stirring at room temperature for 4 h. Using a vacuum rotary evaporator to remove organic solvents from reaction solution, then dissolving again in 150 mL DCM, washing with saturated sodium bicarbonate (2×100 mL), and extracted with saturated saline solution (2×100 mL). Collecting organic layer, drying with anhydrous magnesium sulfate and filtering, then using a vacuum rotary evaporator to remove organic solvent to obtain product G. The product G does not require further purification and can proceed to next reaction step.

### s2: Synthesizing Connecting Group

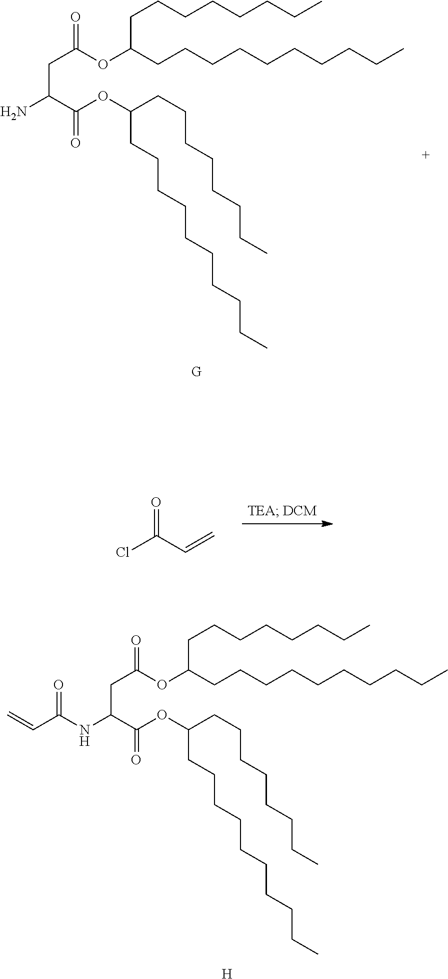

adding 5 mmol of the product G amino acid backbone hydrophobic tail, 7.5 mmol TEA, and 20 mL DCM in a three necked flask containing magnetite. The three necked flask is pre-cooled in an ice bath for 30 minutes; slowly adding dropwise 6.25 mmol of acryloyl chloride (pre-mixed in 10 mL of dichloromethane) dropwise using a constant pressure funnel. After the acryloyl chloride is added dropwise, removing the ice bath. The reaction is left overnight at room temperature, using TLC to detect reaction progress. Wait until the reaction is complete, using DCM (30 mL) to dilute and washing with 1M HCl (50 mL). Collecting organic layer, drying over anhydrous magnesium sulfate and filtering, then using a vacuum rotary evaporator to remove organic solvent. Product is separated by a thin-layer chromatography column (petroleum ether:ethyl acetate=8:1) to obtain target product H with a yield of 88%.

### Hydrogen Spectrum Data of the Obtained Product is:

¹H NMR (400 MHz, CDCl3): 6.69 (m, 1H), 6.32 (d, J=16.8 Hz, 1H), 6.19-6.12 (m, 1H), 5.68 (d, J=10 Hz, 1H), 4.93-4.89 (m, 1H), 4.08-3.93 (m, 4H), 3.10-2.89 (m, 2H), 1.63-1.59 (m, 2H), 1.43-1.26 (m, 66H), 0.88 (t, J=6.4 Hz, 12H).

### s3: Reaction Between Head Group and Tail Group

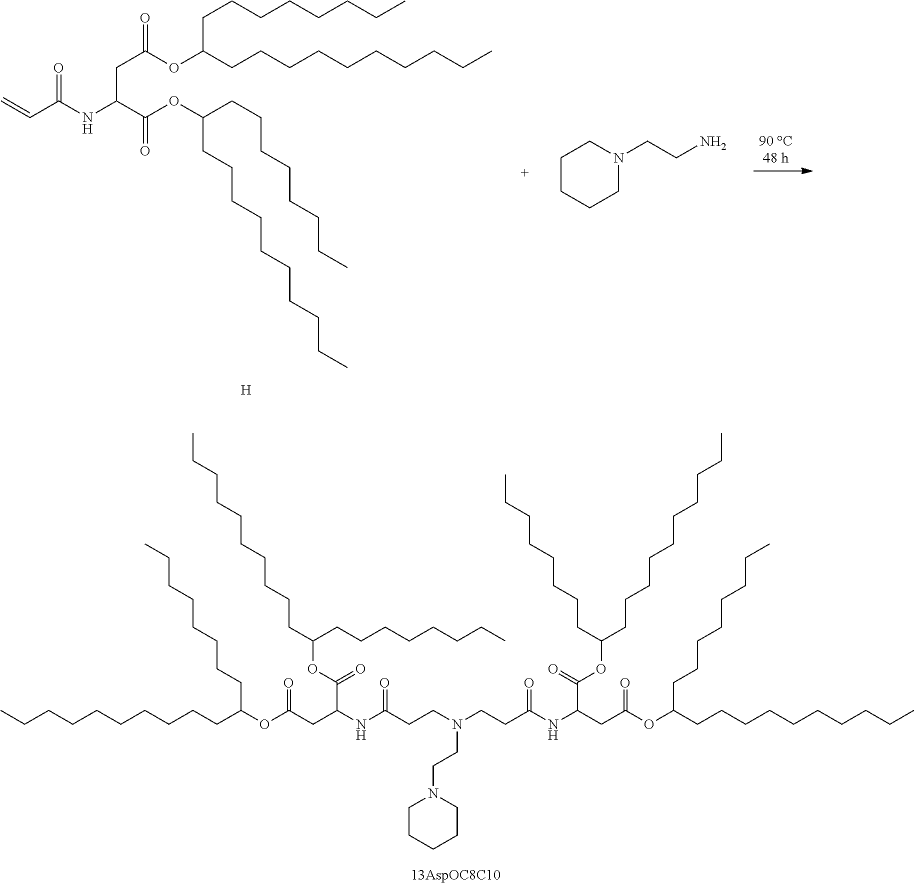

adding 100 mg of 1-(2-aminoethyl) piperidine and 2 times the chemical equivalent of target product H into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 13AspOC8C10.

### Hydrogen Spectrum Data of the Obtained Product is:

1H NMR (400 MHz, CDCl3): 4.78-4.65 (m, 4H), 4.08-4.03 (m, 4H), 3.19-3.02 (m, 8H), 2.81-2.77 (m, 2H), 2.63-2.42 (m, 10H), 1.80-1.48 (m, 20H), 1.39-1.25 (m, 20H), 0.87 (dd, J₁=6.8 Hz, J₂=5.2 Hz, 12H).

### Embodiment 5

Embodiment 5 provides an amino acid backbone ionizable lipid, and a preparation method is:

### s1: Synthesizing Amino Acid Backbone Hydrophobic Tail

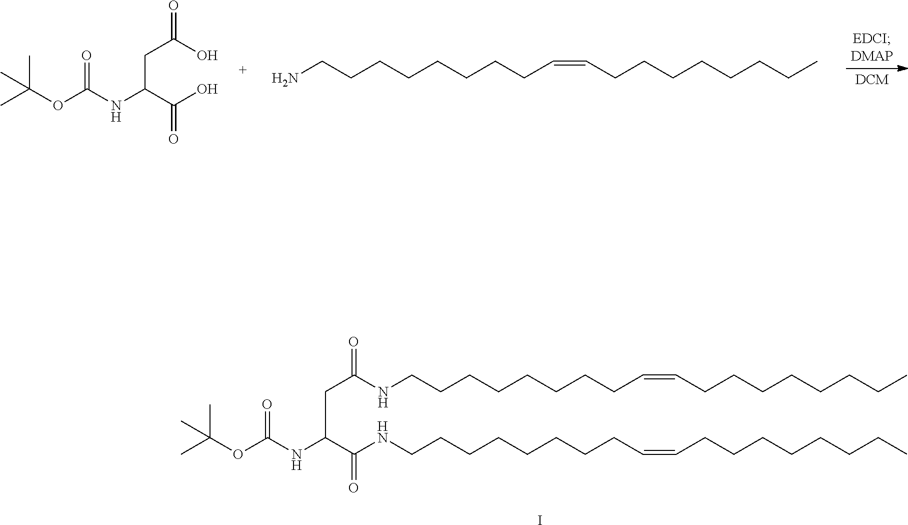

adding 10 mmol BOC-L aspartic acid, 150 mL DCM and magnetite, 22 mmol EDCI, 20 mmol DMAP in a 250 mL reaction flask, stirring at room temperature for 15 minutes, then adding 20 mmol oleylamine and react for 24 hours. Using TLC to detect progress of the reaction. When the reaction is complete, transferring reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent. Product is separated by a thin-layer chromatography column (petroleum ether:ethyl acetate=4:1) to obtain target product I with a yield of 73%.

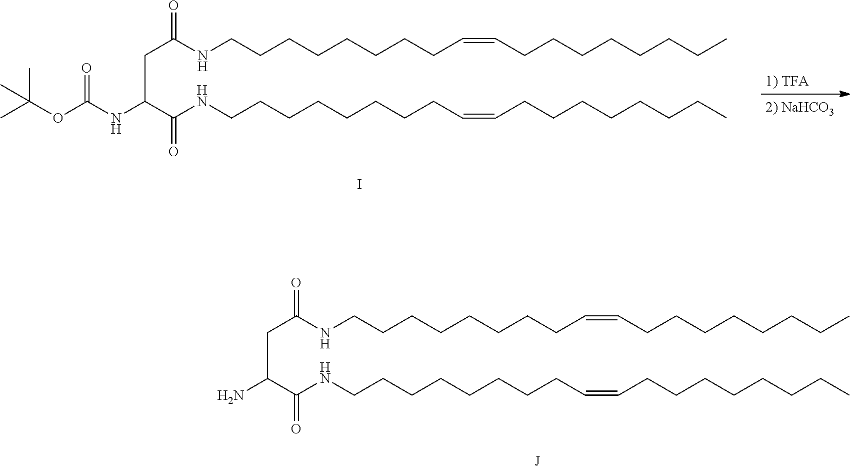

Removing amino protecting group: dissolving the target product F in 30 mL DCM, adding 20 mL TFA and stirring at room temperature for 4 h. Using a vacuum rotary evaporator to remove organic solvents from reaction solution, then dissolving again in 150 mL DCM, washing with saturated sodium bicarbonate (2×100 mL), and extracted with saturated saline solution (2×100 mL). Collecting organic layer, drying with anhydrous magnesium sulfate and filtering, then using a vacuum rotary evaporator to remove organic solvent to obtain product J. The product J does not require further purification and can proceed to next reaction step.

### s2: Synthesizing Connecting Group

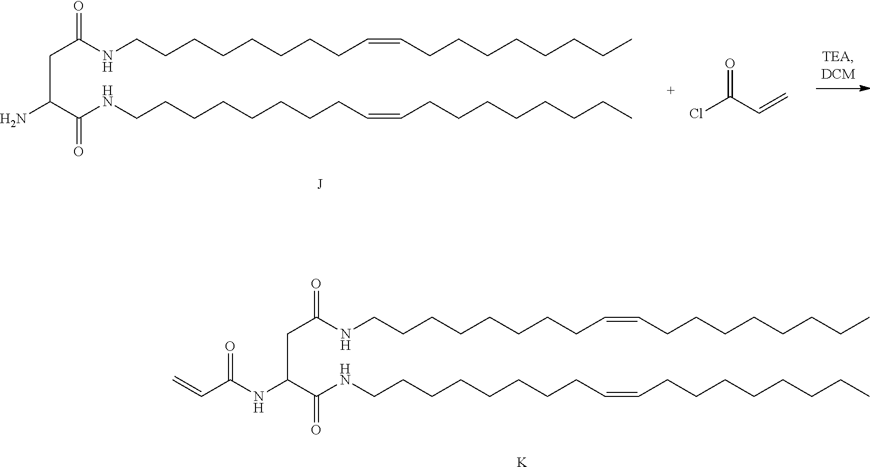

adding 5 mmol of the product G amino acid backbone hydrophobic tail, 7.5 mmol TEA, and 20 mL DCM in a three necked flask containing magnetite. The three necked flask is pre-cooled in an ice bath for 30 minutes; slowly adding dropwise 6.25 mmol of acryloyl chloride (pre-mixed in 10 mL of dichloromethane) dropwise using a constant pressure funnel. After the acryloyl chloride is added dropwise, removing the ice bath. The reaction is left overnight at room temperature, using TLC to detect reaction progress. Wait until the reaction is complete, using DCM (30 mL) to dilute and washing with 1M HCl (50 mL). Collecting organic layer, drying over anhydrous magnesium sulfate and filtering, then using a vacuum rotary evaporator to remove organic solvent. Product is separated by a thin-layer chromatography column (petroleum ether:ethyl acetate=2:1) to obtain target product K with a yield of 86%.

### Hydrogen Spectrum Data of the Obtained Product is:

¹H NMR (400 MHz, CDCl3): 7.88 (d, J=7.2 Hz, 1H), 7.49 (t, J=5.6 Hz, 1H), 6.76 (t, J=5.2 Hz, 1H), 6.33-6.24 (m, 1H), 5.67 (d, J=9.6 Hz, 1H), 5.40-5.33 (m, 4H), 4.82-4.79 (m, 1H), 3.23-3.17 (m, 4H), 2.86-2.81 (m, 1H), 2.57-2.52 (m, 1H), 2.03-2.0 (m, 8H), 1.37-1.18 (m, 48H), 0.88 (dd, J=6.4 Hz, 6H).

### s3: Reaction Between Head Group and Tail Group

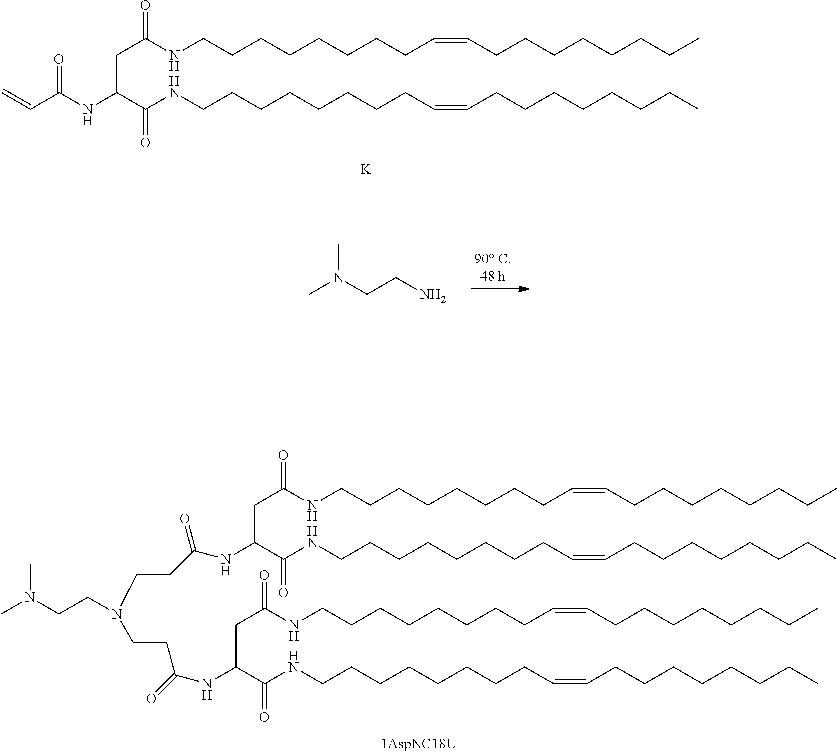

adding 100 mg of 2-(2-methyl-1H-imidazol-1-yl) ethylamine and 2 times the chemical equivalent of target product K into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 1AspNC18U.

### Hydrogen Spectrum Data of the Obtained Product is:

¹H NMR (400 MHz, CDCl3): 4.78-4.65 (m, 4H), 4.08-4.03 (m, 4H), 3.19-3.02 (m, 8H), 2.81-2.77 (m, 2H), 2.63-2.42 (m, 10H), 1.80-1.48 (m, 20H), 1.39-1.25 (m, 20H), 0.87 (dd, J₁=6.8 Hz, J₂=5.2 Hz, 12H).

### Embodiment 6

Embodiment 6 provides an amino acid backbone ionizable lipid, and a preparation method is:

### s1: Synthesizing Amino Acid Backbone Hydrophobic Tail

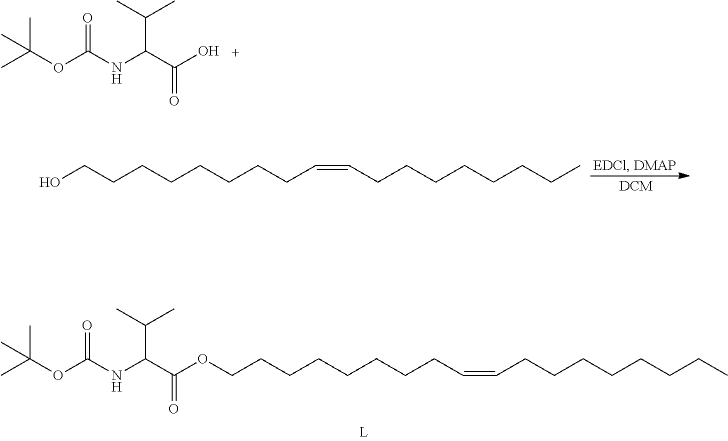

adding 10 mmol BOC-L Valine, 150 mL DCM and magnetite, 11 mmol EDCI, 10 mmol DMAP in a 250 mL reaction flask, stirring at room temperature for 15 minutes, then adding 10 mmol oleyl alcohol and react for 24 hours. Using TLC to detect progress of the reaction. When the reaction is complete, transferring reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent. Product is separated by a thin-layer chromatography column (petroleum ether:ethyl acetate=20:1) to obtain target product L with a yield of 77%.

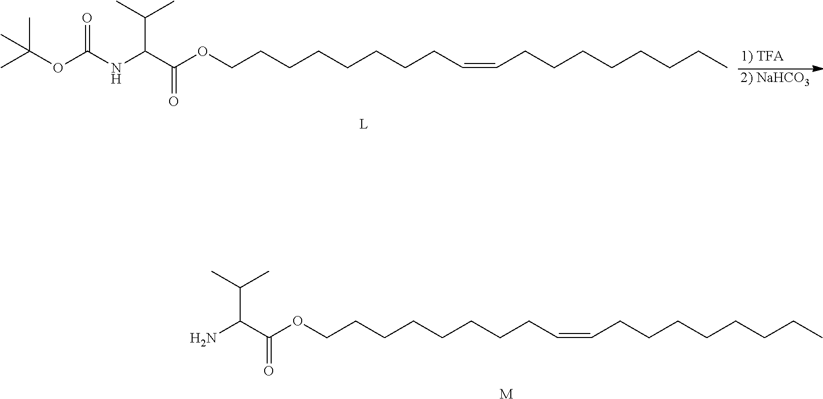

Removing amino protecting group: dissolving the target product L in 30 mL DCM, adding 20 mL TFA and stirring at room temperature for 4 h. Using a vacuum rotary evaporator to remove organic solvents from reaction solution, then dissolving again in 150 mL DCM, washing with saturated sodium bicarbonate (2×100 mL), and extracted with saturated saline solution (2×100 mL). Collecting organic layer, drying with anhydrous magnesium sulfate and filtering, then using a vacuum rotary evaporator to remove organic solvent to obtain product M. The product M does not require further purification and can proceed to next reaction step.

### s2: Synthesizing Connecting Group

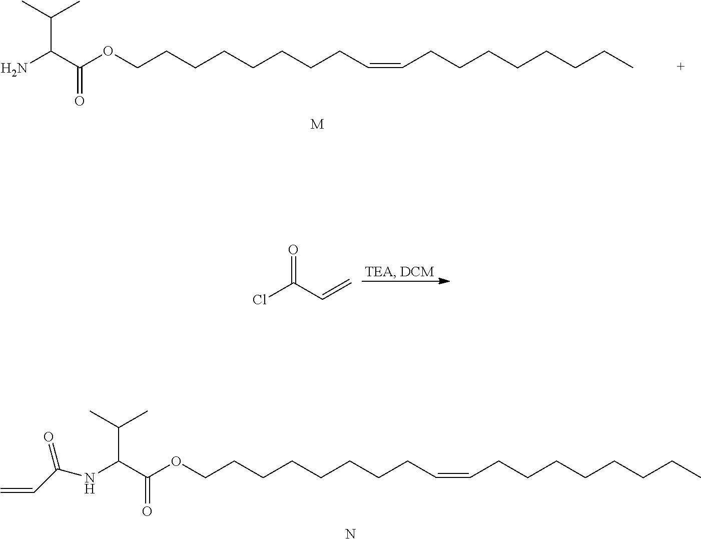

adding 5 mmol of the product M amino acid backbone hydrophobic tail, 7.5 mmol TEA, and 20 mL DCM in a three necked flask containing magnetite. The three necked flask is pre-cooled in an ice bath for 30 minutes; slowly adding dropwise 6.25 mmol of acryloyl chloride (pre-mixed in 10 mL of dichloromethane) dropwise using a constant pressure funnel. After the acryloyl chloride is added dropwise, removing the ice bath. The reaction is left overnight at room temperature, using TLC to detect reaction progress. Wait until the reaction is complete, using DCM (30 mL) to dilute and washing with 1M HCl (50 mL). Collecting organic layer, drying over anhydrous magnesium sulfate and filtering, then using a vacuum rotary evaporator to remove organic solvent. Product is separated by a thin-layer chromatography column (petroleum ether:ethyl acetate=10:1) to obtain target product N with a yield of 98%.

### Hydrogen Spectrum Data of the Obtained Product is:

¹H NMR (400 MHz, CDCl3): 6.41-6.30 (m, 1H), 6.22-6.15 (m, 1H), 5.68-5.66 (m, 1H), 5.41-5.30 (m, 2H), 4.69-4.66 (m, 1H), 4.17-4.10 (m, 1H), 2.23-1.95 (m, 5H), 1.68-1.61 (m, 2H), 1.33-1.26 (m, 22H), 0.97-0.85 (m, 9H).

### s3: Reaction Between Head Group and Tail Group

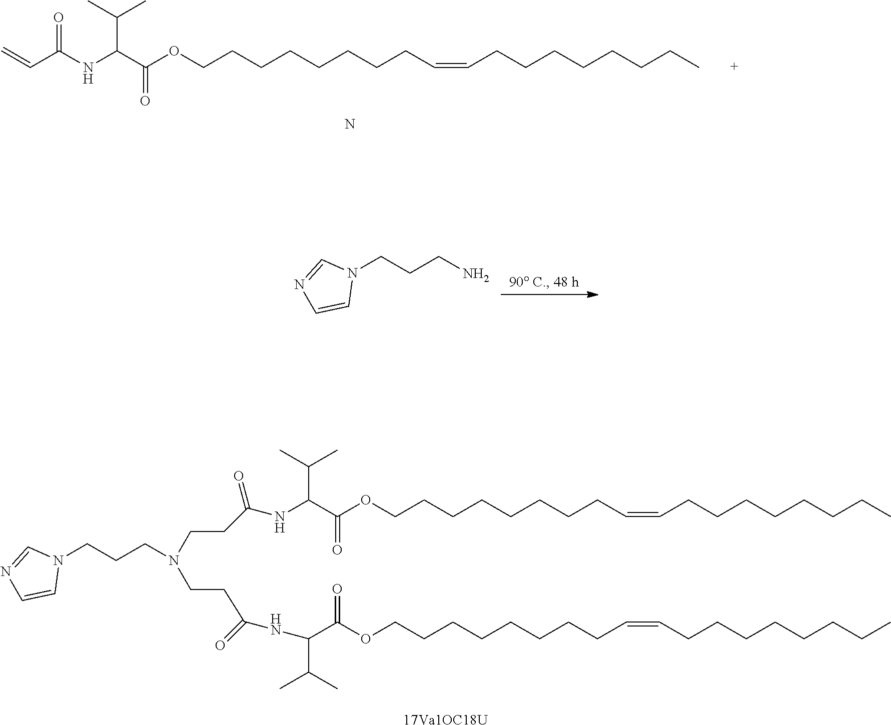

adding 100 mg of 3-aminopropanol and 2 times the chemical equivalent of target product N into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 17ValOC18U.

### Hydrogen Spectrum Data of the Obtained Product is:

¹H NMR (400 MHz, CDCl3): 8.05-7.83 (m, 1H), 7.39-7.37 (m, 1H), 7.21-7.14 (m, 1H), 7.09-7.01 (m, 1H), 5.40-5.34 (m, 4H), 4.58-4.50 (m, 2H), 4.16-4.01 (m, 6H), 3.15-3.10 (q, J=8.0 Hz, 4H), 2.96-2.67 (m, 4H), 2.53-2.40 (m, 4H), 2.23-1.95 (m, 10H), 1.66-1.61 (m, 4H), 1.42-1.26 (m, 44H), 0.97-0.87 (m, 18H).

### Embodiment 7

Embodiment 7 provides an amino acid backbone ionizable lipid, and a preparation method is:

### s1: Synthesizing Amino Acid Backbone Hydrophobic Tail

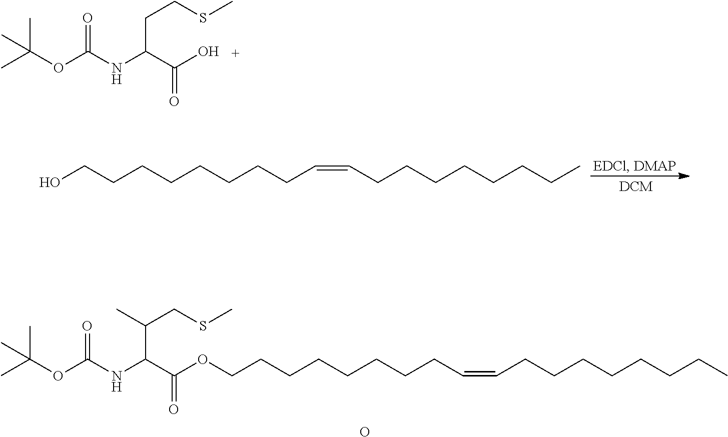

adding 10 mmol BOC-L methionine, 150 mL DCM and magnetite, 11 mmol EDCI, 10 mmol DMAP in a 250 mL reaction flask, stirring at room temperature for 15 minutes, then adding 10 mmol oleyl alcohol and react for 24 hours. Using TLC to detect progress of the reaction. When the reaction is complete, transferring reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent. Product is separated by a thin-layer chromatography column (petroleum ether:ethyl acetate=20:1) to obtain target product O with a yield of 64%.

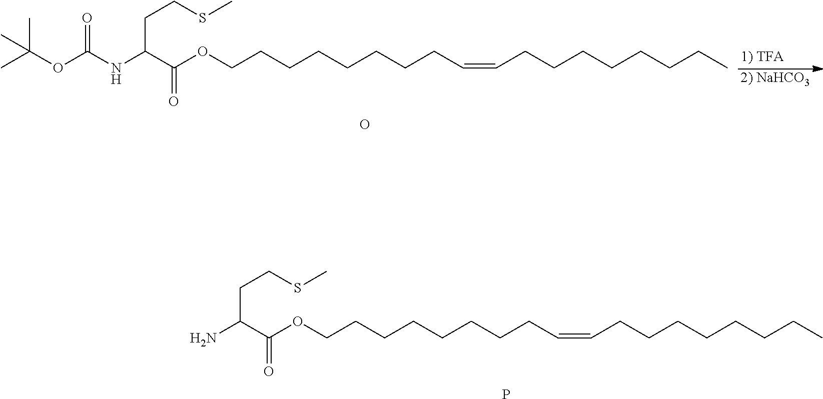

Removing amino protecting group: dissolving the target product O in 30 mL DCM, adding 15 mL TFA and stirring at room temperature for 4 h. Using a vacuum rotary evaporator to remove organic solvents from reaction solution, then dissolving again in 150 mL DCM, washing with saturated sodium bicarbonate (2×100 mL), and extracted with saturated saline solution (2×100 mL). Collecting organic layer, drying with anhydrous magnesium sulfate and filtering, then using a vacuum rotary evaporator to remove organic solvent to obtain product P. The product P does not require further purification and can proceed to next reaction step.

### s2: Synthesizing Connecting Group

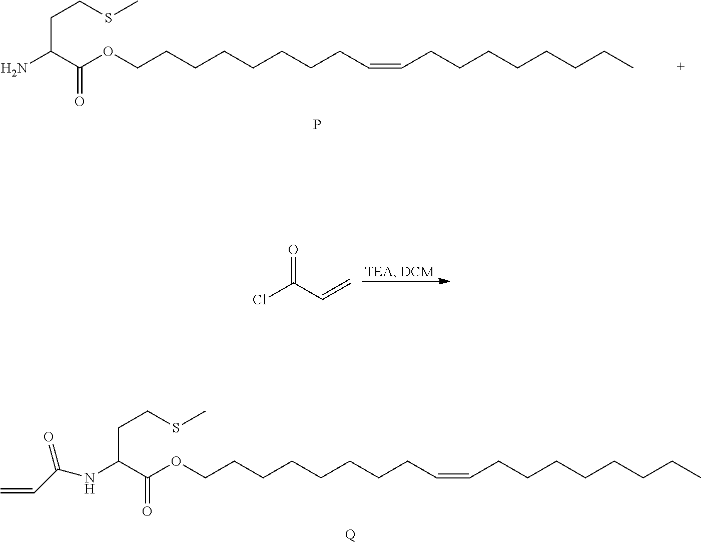

adding 5 mmol of the product M amino acid backbone hydrophobic tail, 7.5 mmol TEA, and 20 mL DCM in a three necked flask containing magnetite. The three necked flask is pre-cooled in an ice bath for 30 minutes; slowly adding dropwise 6.25 mmol of acryloyl chloride (pre-mixed in 10 mL of dichloromethane) dropwise using a constant pressure funnel. After the acryloyl chloride is added dropwise, removing the ice bath. The reaction is left overnight at room temperature, using TLC to detect reaction progress. Wait until the reaction is complete, using DCM (30 mL) to dilute and washing with 1M HCl (50 mL). Collecting organic layer, drying over anhydrous magnesium sulfate and filtering, then using a vacuum rotary evaporator to remove organic solvent. Product is separated by a thin-layer chromatography column (petroleum ether:ethyl acetate=10:1) to obtain target product Q with a yield of 88%.

### Hydrogen Spectrum Data of the Obtained Product is:

¹H NMR (400 MHz, CDCl3): 6.34-6.30 (m, 1H), 6.20-6.13 (m, 1H), 5.70-5.67 (m, 1H), 5.40-5.30 (m, 2H), 4.82-4.77 (m, 2H), 4.16-4.11 (m, 1H), 2.59-2.47 (m, 2H), 2.26-1.94 (m, 9H), 1.68-1.61 (m, 2H), 1.34-1.23 (m, 22H), 0.89-0.85 (m, 3H).

### s3: Reaction Between Head Group and Tail Group

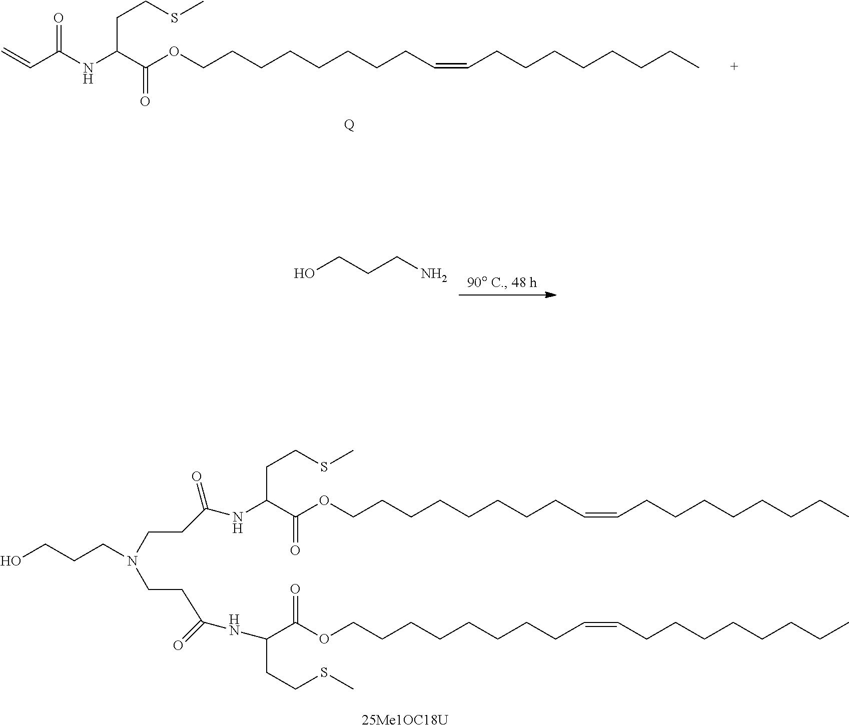

adding 100 mg of 3-aminopropanol and 2 times the chemical equivalent of target product Q into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 17ValOC18U.

### Hydrogen Spectrum Data of the Obtained Product is:

¹H NMR (400 MHz, CDCl3): 7.77-7.69 (m, 2H), 5.41-5.35 (m, 4H), 4.69-4.62 (m, 2H), 4.15-4.11 (m, 4H), 3.80-3.76 (m, 2H), 3.18-3.10 (m, 4H), 2.84-2.71 (m, 4H), 2.62-2.57 (m, 4H), 2.14-1.97 (m, 20H), 1.68-1.64 (m, 4H), 1.45-1.27 (m, 44H), 0.91-0.88 (m, 6H).

### Embodiment 8

Embodiment 8 provides an amino acid backbone ionizable lipid, and a preparation method is:

### s1: Synthesizing Amino Acid Backbone Hydrophobic Tail

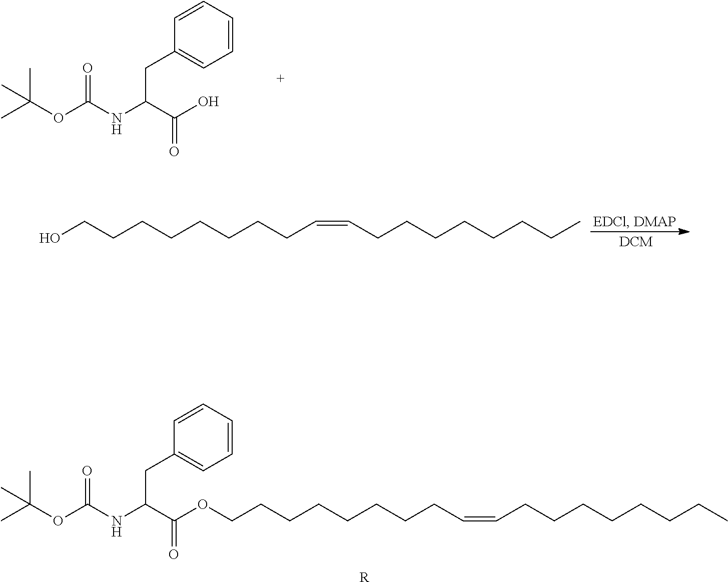

adding 10 mmol BOC-L phenylalanine, 150 mL DCM and magnetite, 11 mmol EDCI, 10 mmol DMAP in a 250 mL reaction flask, stirring at room temperature for 15 minutes, then adding 10 mmol oleyl alcohol and react for 24 hours. Using TLC to detect progress of the reaction. When the reaction is complete, transferring reaction mixture into a separatory funnel, adding DCM (2×100 mL) washing with 1M HCl (2×100 mL), and extracted with saturated saline solution (2×200 mL). Collecting organic layer, drying with anhydrous magnesium sulfate and filtering, then using a vacuum rotary evaporator to remove organic solvent. Product is separated by a thin-layer chromatography column (petroleum ether:ethyl acetate=20:1) to obtain target product R with a yield of 97%.

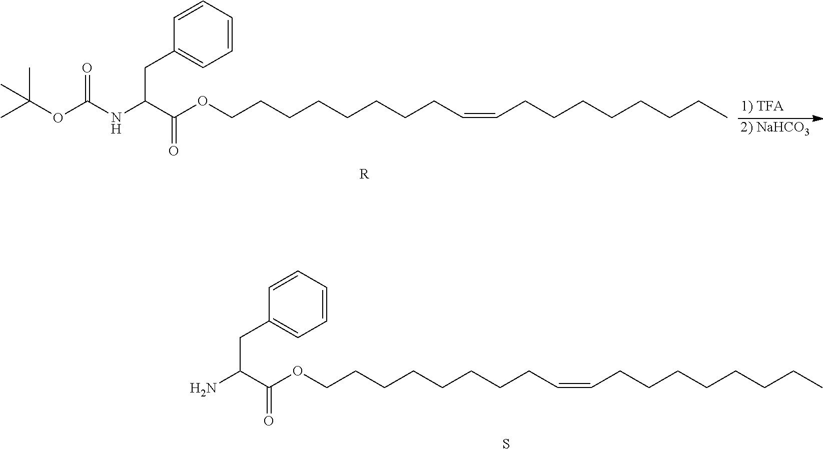

Removing amino protecting group: dissolving the target product R in 30 mL DCM, adding 15 mL TFA and stirring at room temperature for 4 h. Using a vacuum rotary evaporator to remove organic solvents from reaction solution, then dissolving again in 150 mL DCM, washing with saturated sodium bicarbonate (2×100 mL), and extracted with saturated saline solution (2×100 mL). Collecting organic layer, drying with anhydrous magnesium sulfate and filtering, then using a vacuum rotary evaporator to remove organic solvent to obtain product S. The product S does not require further purification and can proceed to next reaction step.

### s2: Synthesizing Connecting Group

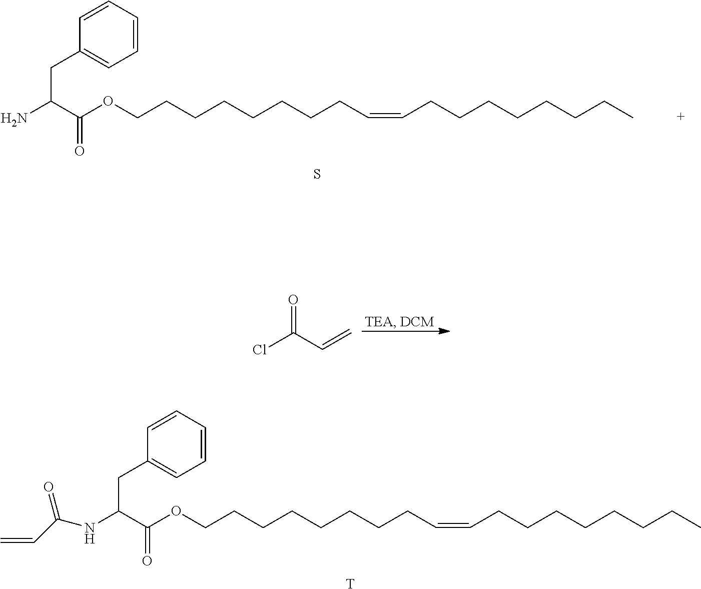

adding 5 mmol of the product S amino acid backbone hydrophobic tail, 7.5 mmol TEA, and 20 mL DCM in a three necked flask containing magnetite. The three necked flask is pre-cooled in an ice bath for 30 minutes; slowly adding dropwise 6.25 mmol of acryloyl chloride (pre-mixed in 10 mL of dichloromethane) dropwise using a constant pressure funnel. After the acryloyl chloride is added dropwise, removing the ice bath. The reaction is left overnight at room temperature, using TLC to detect reaction progress. Wait until the reaction is complete, using DCM (30 mL) to dilute and washing with 1M HCl (50 mL). Collecting organic layer, drying over anhydrous magnesium sulfate and filtering, then using a vacuum rotary evaporator to remove organic solvent. Product is separated by a thin-layer chromatography column (petroleum ether:ethyl acetate=10:1) to obtain target product T with a yield of 69%.

### Hydrogen Spectrum Data of the Obtained Product is:

¹H NMR (400 MHz, CDCl3): 7.31-7.11 (m, 5H), 6.33-6.27 (m, 1H), 6.16-6.09 (m, 1H), 5.68-5.65 (m, 1H), 5.41-5.35 (m, 2H), 4.99-4.95 (m, 1H), 4.15-4.08 (m, 2H), 3.19-3.16 (m, 2H), 2.06-2.03 (m, 4H), 1.63-1.59 (m, 2H), 1.37-1.28 (m, 22H), 0.91-0.87 (m, 3H).

### s3: Reaction Between Head Group and Tail Group

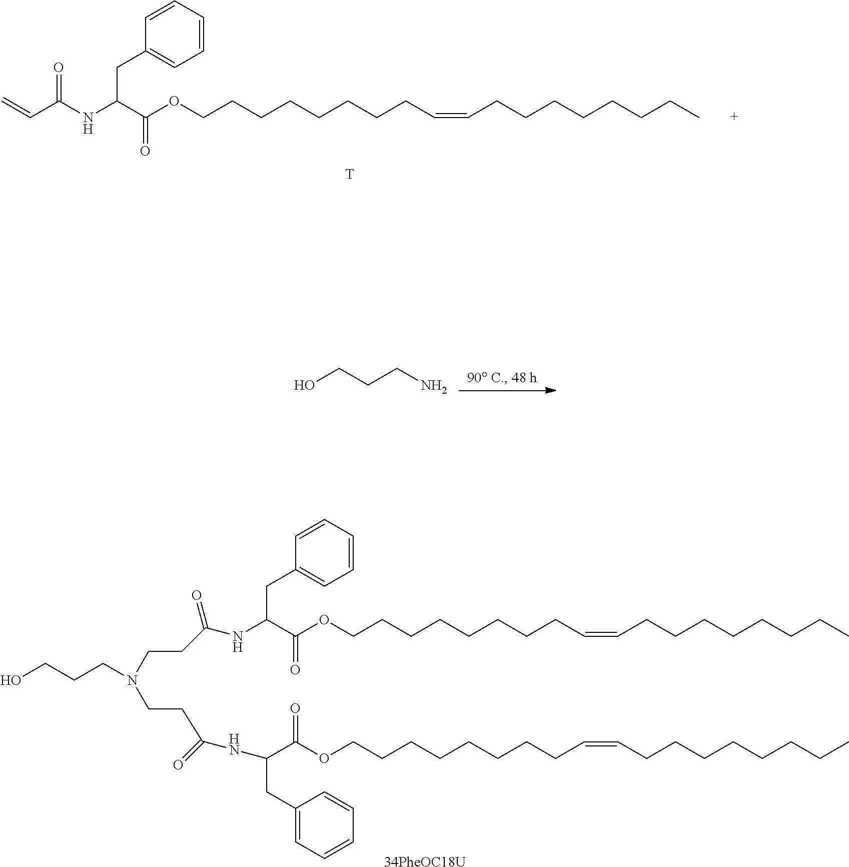

adding 100 mg of 3-aminopropanol and 2 times the chemical equivalent of target product T into a 5 mL reaction bottle containing magnetite (with a tetrafluoroethylene lining on a bottle cap), react at 90° C. for 48 hours. After the reaction is complete, product is separated by a thin-layer chromatography column (DCM:methanol=20:1) to obtain ionizable lipid 34PheOC18U.

### Hydrogen Spectrum Data of the Obtained Product is:

1H NMR (400 MHz, CDCl3): 7.31-7.16 (m, 10H), 5.41-5.35 (m, 4H), 4.84-4.80 (m, 2H), 4.10-4.06 (m, 4H), 3.66-3.63 (m, 4H), 3.66-3.05 (m, 6H), 2.95-2.94 (m, 2H), 2.65-2.52 (m, 4H), 2.08-1.96 (m, 8H), 1.62-1.53 (m, 6H), 1.45-1.27 (m, 48H), 0.91-0.88 (m, 6H).

The amino acid backbone ionizable lipid synthesized by the present disclosure is (refer to general preparation method and Embodiments 1-8 for a preparation method of the amino acid backbone ionizable lipid with other structures):1GluOC8, 1AspOC8, 1GluOC10, 1AspOC10, 1GluOC8C10, 1AspOC8C10, 1GluOC10, 1AspOC10, 1GluOC12, 1AspOC12, 1GluOC16, 1AspOC16, 1GluOC18, 1AspOC18, 1GluOC18U, 1AspOC18U, 1GluOC18U2, 1AspOC18U2, 1GluNC8, 1AspNC8, 1GluNC18U, 1AspNC18U, 3GluOC18U, 3AspOC18U, 4GluOC18U, 4AspOC18U, 1LeuOC18U, 1LeuNC18U, 6AspOC18U, 6GluOC18U, 8GluOC18, 8AspOC18, 8GluOC18U, 9GluOC8, 8AspOC18U, 9AspOC8, 9GluOC10, 9AspOC10, 9GluOC18U, 9AspOC18U, 9GluOC18U2, 9AspOC18U2, 11GluOC8, 11AspOC8, 11GluOC10, 11AspOC10, 12GluOC18U, 12AspOC18U, 13AspOC8, 13AspOC10, 13GluOC8C10, 13AspOC8C10, 16AspOC10, 16GluOC18U2, 16GluOC18U, 16AspOC18U, 17AspOC10, 17GluOC18U2, 17GluOC18U, 17AspOC18U, 18GluOC18U, 18AspOC18U, 19GluOC8C10, 19AspOC8C10, 19GluOC18U, 19AspOC18U, 20GluOC18U, 20AspOC18U, 21GluOC18U, 21GluNC18U, 22GluOC18U, 22AspOC18U, 24GluOC18U, 24AspOC18U, 25GluOC18U, 25AspOC18U, 26GluOC18U, 26AspOC18U, 27GluOC18U, 27AspOC18U, 28GluOC18U, 28AspOC18U, 29GluOC18U, 29AspOC18U, 30GluOC18U, 30AspOC18U, 31GluOC18U, 31AspOC18U, 32GluOC18U, 32GluNC18U, 33GluOC18U, 33AspOC18U, 34GluOC18U, 34AspOC18U, 17PheOC18, 17MetOC18U, 34ValOC18U, 17GayOC18U, 29PheOC18U, 29MetOC18U, 29ValOC18U, 29GayOC18U, 34PheOC18U, 34MetOC18U, 34ValOC18U, 34GayOC18U.

### Characterization of Lipid Experiments

1. Characterization of Enzyme Delivery Efficiency of Fluorescent Proteins and fluoresceinR₁ is Selected from

Validating efficiency of LNP containing ionizable lipids in delivering plasmids encoding Green Fluorescent Protein (GFP) and Firefly Luciferase (Luc) (DNA-GFP-Luc) in 293T cell lines. Using ionizable lipids 1GluOC18, 8GluOC18, 3GluOC18U, 4GluOC18U, 12GluOC18U, 13GluOC18U, 17GluO18U, 1AspOC18U, 3AspOC18U, 1AspOC8, 1AspOC10, 8GluOC18U, 9AspOC8, 9AspOC10, 9GluOC18U, 13AspOC8, 13AspOC10, 13AspOC8C10, 11AspOC8, 11AspOC10, 16AspOC10, 16AspOC18U, 17AspOC10, 17AspOC18U, 19AspOC18U, 19AspOC8C10, 22AspOC18U, 21GluNC18U, 16GluOC18U, 20GluOC18U, 21GluOC18U, 1GluOC18U2, 9GluOC18U2, 16GluOC18U2, 17GluOC18U2, 9GluOC18U, 16GluOC18U, 19GluOC18U, 25GluOC18U, 26GluOC18U, 27GluOC18U, 29GluOC18U, 30GluOC18U, 31GluOC18U, 32GluOC18U, 33GluOC18U, 34GluOC18U, 17GayOC18U, 29GayOC18U, 34GayOC18U, 17PheOC18U, 29PheOC18U, 34PheOC18U, 17ValOC18U, 29ValOC18U, 34ValOC18U, 17MetOC18U, 29MetOC18U, 34MetOC18U and commercial materials ALC-0315 and SM-102 as delivery materials to express DNA-GFP-Luc in cells.

### Specific Steps are:

s1: Cell culture: One day before the experiment, seeding the cultured 293T cells in a 96 well cell culture plate. When cell density grow to 70-80%, performing a cell transfection experiment.
s2: preparation of lipid nanoparticles LNP-DNA-GFP-Luc for cell transfection
dissolving the ionizable lipid and DSPC, cholesterol, DSPE-PEG in anhydrous ethanol at concentrations of 10 mg/mL, 3 mg/mL, 6 mg/mL, and 1 mg/mL, respectively. Mixing evenly in a molar ratio of ionizable lipid: cholesterol:DSPC:DSPE-PEG=40:48:10:2 to obtain lipid mixture solution. At the same time, taking an appropriate amount of DNA-GFP-Luc and dissolving in sodium citrate buffer (a volume of sodium citrate buffer is three times the total volume of the lipid mixture, pH=4.0-4.5) to obtain DNA buffer. Then quickly mixing the DNA buffer with the lipid mixture solution, incubated at room temperature for 15 minutes, assembling a stable LNP (transfection of LNP containing 150 ng DNA-GFP-Luc in each well). Diluted with twice the volume of sterile PBS and added to the 96 well cell culture plate for transfection. Where a nitrogen to phosphorus ratio between ionizable lipids and nucleic acids is 6:1, which is a molar ratio between protonated amino groups and phosphate groups on nucleic acids. (Same below)

Positive control group: using commercial lipid ALC-0315 and SM-102 to assemble LNP according to publicly available preparation method. Specific operation is as follows: dissolving ALC-0315 or SM-102, DSPC, cholesterol, ALC-0159 or DMG-PEG2000 in anhydrous ethanol at concentrations of 5 mg/mL, 1.5 mg/mL, 3 mg/mL, and 1 mg/mL respectively. Mixing at a uniform molar ratio of ALC-0315: cholesterol:DSPC:ALC-0159=46.3:42.7:9.4:1.6 or SM-102: cholesterol:DSPC:DMG-PEG2000=50:38.5:10:1.5. At the same time, taking an appropriate amount of DNA-GFP-Luc and dissolving in sodium citrate buffer (a volume of sodium citrate buffer is three times to the total volume of the lipid mixture, pH=4.0), then rapidly mixing the DNA buffer with the lipid mixture solution and incubated at room temperature for 15 minutes to assemble stable LNP (transfection of LNP containing 150 ng DNA-GFP-Luc per well). Diluting the LNP with twice the volume of sterile PBS and adding to 96 well cell culture plates for transfection. A nitrogen to phosphorus ratio of ALC-0315 and nucleic acid is 6:1, a nitrogen to phosphorus ratio of SM-102, and nucleic acid is 6:1.

Negative control group: culturing 293T cells normally and adding unencapsulated DNA-GFP-Luc.

### s3: Analysis of Cell Transfection Efficiency

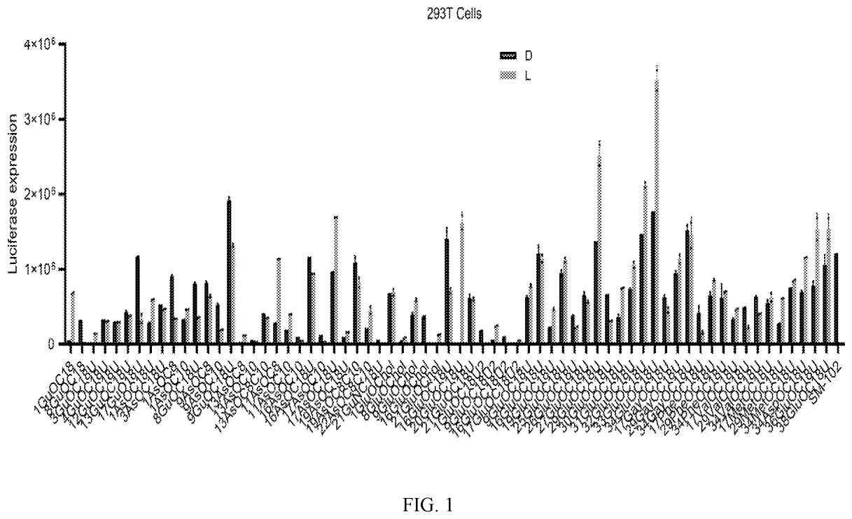

after 36 hours of cell transfection, detecting expression of green fluorescent protein with a fluorescence microscope. Sucking up culture medium of the 96 well cell culture plate, adding cell lysis buffer and lyse the cells on ice for 30 minutes. After centrifugation, taking supernatant and transferring to a white 96 well detection plate. Adding firefly luciferase substrate and using an enzyme-linked immunosorbent assay (ELISA) reader to detect firefly luciferase content (chemiluminescence). Relative luciferase activity results are shown in FIG. 1. The results shows that the ionizable lipid synthesized by the present disclosure can greatly enhance the transfection efficiency of nucleic acids. When a tail of ionizable lipid is GluO18U or AspOC18U, a RNA expression efficiency is highest; when a tail is GluO8 or AspOC8, a RNA expression efficiency is weak. Lipid transfection efficiency represented by 29GluO18U, 34GluO18U, and 17AspOC18U is superior to commercial lipids ALC-0315 and SM-102, with an efficiency increase of about 2-3 times, verifying rationality and high efficiency of the overall chemical structure of the ionizable lipid designed by the present disclosure.

### 2. LNP Component Optimization Experiment

Validating efficiency of delivering saRNA-GFP-Luc using LNP containing ionizable lipids in 293T cell lines. Optimizing composition of LNP using ionizable lipid 17LGluO18U.

### Specific Steps are:

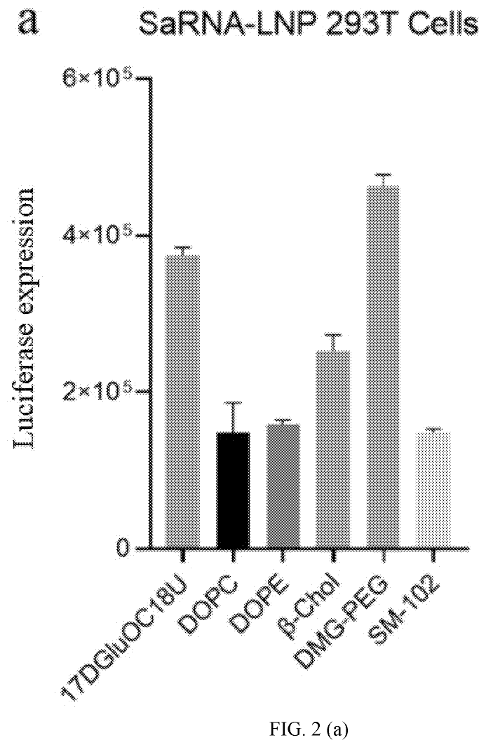

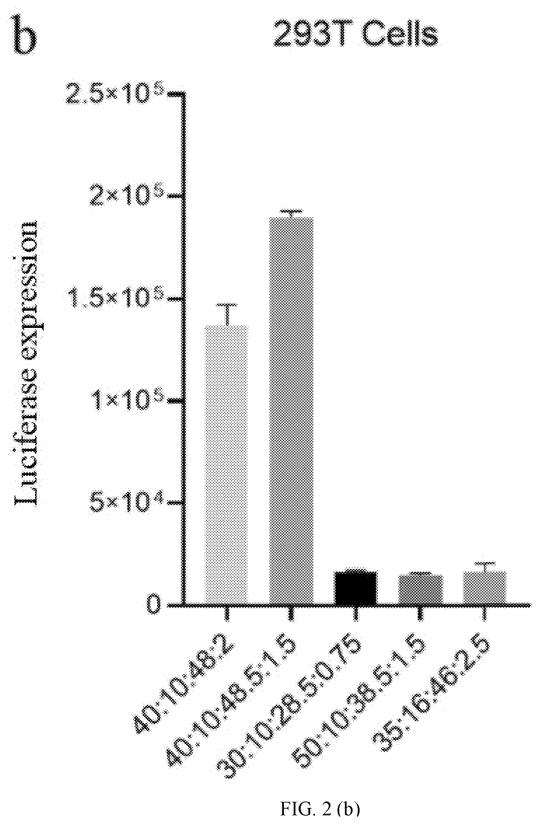

s1: Referring to the above experimental method, an ionizable lipid used in experimental group is 17GluO18U. Dissolving ionizable lipid, DOPE or DSPC or DOPC, cholesterol or 0-sitosterol, DSPE-PEG or DMG-PEG in anhydrous ethanol at concentrations of 10 mg/mL, 3 mg/mL, 6 mg/mL, and 1 mg/mL, respectively. A usage ratio is ionizable lipid 17LGuO18U: (cholesterol or β-sitosterol): (DOPE or DSPC or DOPC): (DSPE-PEG or DMG-PEG)=40:48:10:2.
   s2: analysis of Cell Transfection Efficiency
after 36 hours of cell transfection, detecting expression of green fluorescent protein with a fluorescence microscope. Sucking up culture medium of the 96 well cell culture plate, adding cell lysis buffer and lyse the cells on ice for 30 minutes. After centrifugation, taking supernatant and transferring to a white 96 well detection plate. Adding firefly luciferase substrate and using an enzyme-linked immunosorbent assay (ELISA) reader to detect firefly luciferase content (chemiluminescence). Relative luciferase activity results are shown in FIG. 2 (a). The results shows that chemical structure of neutral auxiliary phospholipid greatly affects RNA delivery efficiency. When the neutral auxiliary phospholipid is DSPC, the delivery efficiency of the three ionizable lipids is significantly better than the neutral auxiliary phospholipid is DOPE or DOPC, so DSPC is the preferred neutral auxiliary phospholipid. cholesterol is preferred over β-sitosterol. A chain length of PEG significantly affects the delivery efficiency of RNA, and DMG-PEG with shorter chain length is preferred.

### 3. Optimization Experiment of LNP Component Ratio

Validating efficiency of delivering saRNA-GFP-Luc using LNP containing ionizable lipids in 293T cell lines. Optimizing proportion of each component in LNP using ionizable lipid 17LGluO18U.

### Specific Steps are:

s1: Referring to the above experimental method, an ionizable lipid used in experimental group is 17LGluO18U. Dissolving ionizable lipid, DSPC, cholesterol,DSPE-PEG in anhydrous ethanol at concentrations of 10 mg/mL, 3 mg/mL, 6 mg/mL, and 1 mg/mL, respectively. Mixing six different molar ratios, where ratio A is ionizable lipid: cholesterol:DSPC:DMG-PEG=40:48:10:2; ratio B is ionizable lipid: cholesterol:DSPC:DMG-PEG=30:28.5:10:0.75; ratio C is ionizable lipid: cholesterol:DSPC:DMG-PEG=50:38.5:10:1.5; ratio D is ionizable lipid: cholesterol:DSPC:DMG-PEG=35:46:16:2.5; ratio E is ionizable lipid: cholesterol:DSPC:DMG-PEG=46.3:42.7:9.4:1.6; ratio F is ionizable lipid: cholesterol:DSPC:DMG-PEG=46.3:42.7:9.4:1.5.

### s2: Analysis of Cell Transfection Efficiency

after 36 hours of cell transfection, detecting expression of green fluorescent protein with a fluorescence microscope. Sucking up culture medium of the 96 well cell culture plate, adding cell lysis buffer and lyse the cells on ice for 30 minutes. After centrifugation, taking supernatant and transferring to a white 96 well detection plate. Adding firefly luciferase substrate and using an ELISA reader to detect firefly luciferase content (chemiluminescence). Relative luciferase activity results are shown in FIG. 2 (b). The results shows that a molar ratio between the components of LNP also affects RNA delivery efficiency to a certain extent, and an optimal ratio F is ionizable lipid: cholesterol:DSPC:DMG-PEG=40:48:10:1.5.

### 4. Optimization Experiment of LNP Nitrogen Phosphorus Ratio

Validating efficiency of delivering saRNA-GFP-Luc using LNP containing ionizable lipids in 293T cell lines. Optimizing nitrogen phosphorus ratio in LNP using ionizable lipid 17LGluO18U.

### Specific Steps are:

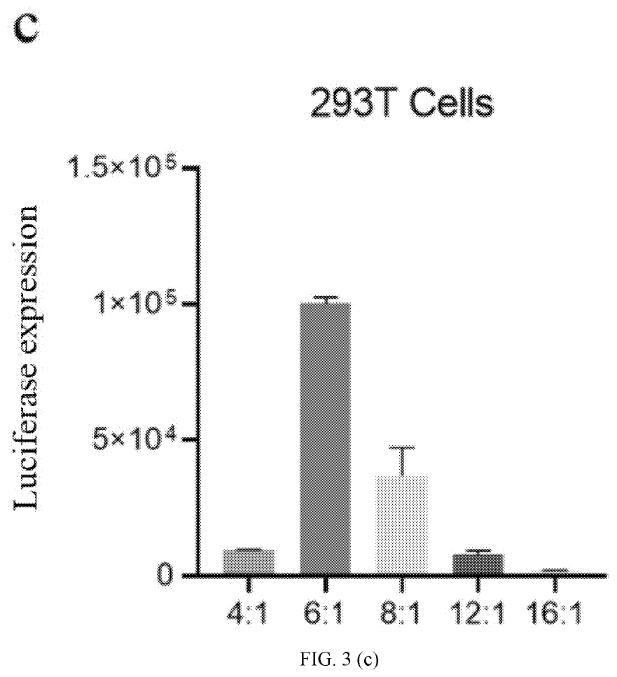

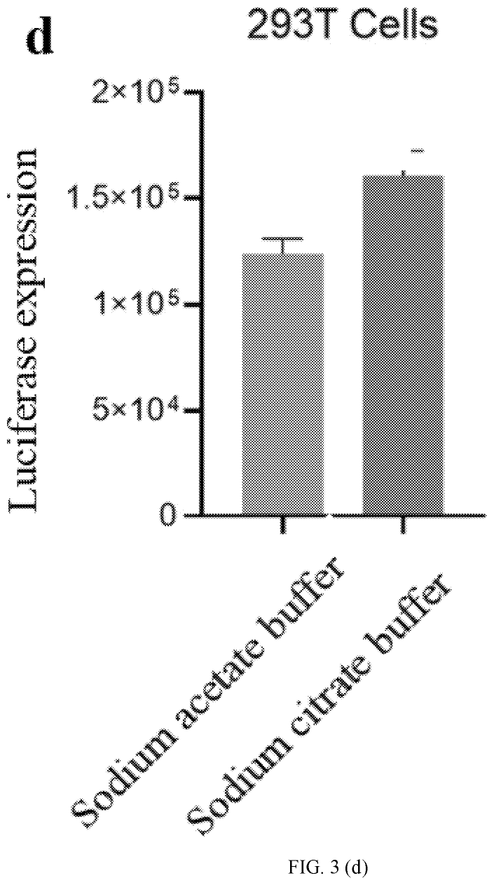

s1: Referring to the above experimental method, an ionizable lipid used in experimental group is 17GluO18U. Dissolving ionizable lipid, DSPC, cholesterol, DMG-PEG in anhydrous ethanol at concentrations of 10 mg/mL, 3 mg/mL, 6 mg/mL, and 1 mg/mL, a usage ratio is ionizable lipid 17GluO18U: cholesterol:DSPC:DMG-PEG=40:48:10:1.5. Where nitrogen phosphorus ratio of LNP in group using ionizable lipid are 4:1, 6:1, 8:1, 12:1, 16:1.
s2: analysis of cell transfection efficiency
after 36 hours of cell transfection, detecting expression of green fluorescent protein with a fluorescence microscope. Sucking up culture medium of the 96 well cell culture plate, adding cell lysis buffer and lyse the cells on ice for 30 minutes. After centrifugation, taking supernatant and transferring to a white 96 well detection plate. Adding firefly luciferase substrate and using an ELISA reader to detect firefly luciferase content (chemiluminescence). Relative luciferase activity results are shown in FIG. 3 (a). Results shows that transfection efficiency of saRNA-GFP-Luc is optimal when nitrogen phosphorus ratio is 6:1.

### 5. Optimization Experiment of LNP Buffer Formula

Validating efficiency of delivering saRNA-GFP-Luc using LNP containing ionizable lipids in 293T cell lines. Optimizing buffer formula in LNP using ionizable lipid 17LGluO18U.

### Specific Steps are:

s1:Referring to the above experimental method, an ionizable lipid used in experimental group is 17LGluO18U. Dissolving ionizable lipid, DSPC, cholesterol, DMG-PEG in anhydrous ethanol at concentrations of 10 mg/mL, 3 mg/mL, 6 mg/mL, and 1 mg/mL, a usage ratio is ionizable lipid 17LGluO18U, 34LGluO18U, or 16AspOC18U: cholesterol:DSPC:DMG-PEG=40:48:10:1.5. Where solution of pre-mixed RNA is sodium acetate buffer or sodium citrate buffer, nitrogen phosphorus ratio for preparing LNP is 6:1.
s2: analysis of cell transfection efficiency
after 36 hours of cell transfection, detecting expression of green fluorescent protein with a fluorescence microscope. Sucking up culture medium of the 96 well cell culture plate, adding cell lysis buffer and lyse the cells on ice for 30 minutes. After centrifugation, taking supernatant and transferring to a white 96 well detection plate. Adding firefly luciferase substrate and using an ELISA reader to detect firefly luciferase content (chemiluminescence). Relative luciferase activity results are shown in FIG. 3 (b). The results shows that when sodium citrate buffer is used to prepare nanoparticles, RNA delivery efficiency is better than that of sodium acetate buffer. Therefore, sodium citrate buffer is preferred.
   6. Characterization of Delivery Efficiency of modRNA-GFP

Validating efficiency of LNP delivery of modRNA-GFP containing ionizable lipids in Jurkat T cell lines. Verifying transfection efficiency of LNP using ionizable lipids: 9LGluOC18U, 17LGluO18U, 25DGluO18U, 29LGluO18U.

### Specific Steps are:

s1: Referring to the above experimental method, Juekat T cell line is used, and ionizable lipids used in the experimental group are: 9LGluOC18U, 17LGluO18U, 25DGluO18U, 29LGluO18U. Ionizable lipids used in control group are SM-10 and ALC-0315, other components of LNP are DSPC, cholesterol, and DMG-PEG. Dissolving the other components of LNP in anhydrous ethanol at concentrations of 10 mg/mL, 3 mg/mL, 6 mg/mL, and 1 mg/mL, a usage ratio ionizable lipid compound: Cholesterol:DSPC:DMG-PEG=40:48:1.5. Where solution of pre-mixed modRNA is sodium citrate buffer, and a nitrogen phosphorus ratio for preparing LNP is 6:1.

### s2: Analysis of Cell Transfection Efficiency

After transfection for 24 hours, detecting expression of green fluorescent protein using a fluorescence microscope. Results are shown in FIG. 4, indicating that the ionizable lipid of the present disclosure can efficiently deliver nucleic acids to immune cells and is superior to commercial lipids.

### 7. Characterization of In Vivo Imaging System

Using LNP containing ionizable lipids 9GluOC18U, 17GluOC18U, 25GluOC18U, 29GluOC18U, 33GluOC18U, 34GluOC18U, and commercial lipids SM-102 and ALC-0315 to deliver chemically synthesized modified messenger ribonucleotides (modRNA-Luc) encoding firefly luciferase in Balb/c mice. After intravenous injection for 6 hours, detecting and reporting a expression of gene luciferase using an in vivo imaging system (IVIS).

### Specific Steps are:

s1: Referring to the above experimental method, ionizable lipids used in the experimental group are 9GluOC18U, 17GluOC18U, 25GluOC18U, 29GluOC18U, 33GluOC18U, and 34GluOC18U. Dissolving ionizable lipids, DSPC, cholesterol, DMG-PEG in anhydrous ethanol at concentrations of 10 mg/mL, 6 mg/mL, 12 mg/mL, and 5 mg/mL. Taking an appropriate amount of modRNA-Luc and dissolving in sodium acetate buffer (a volume of sodium acetate buffer is twice the total volume of lipid mixture, pH=4.0). Taking the sodium acetate buffer containing modRNA-Luc and adding to lipid mixture ethanol solution, quickly mixing evenly to assemble LNP to obtain mixed solution. Incubating the mixed solution at room temperature for 15 minutes, dialyzing in PBS using a dialysis bag (MWCO=14000MW) for 1 hour, and then performing tail vein injection (each injection containing 5 g modRNA Luc of LNP). A usage ratio is ionizable lipids 9GluOC18U, 17GluOC18U, 25GluOC18U, 29GluOC18U, 33GluOC18U, or 34 GluOC18U: cholesterol:DSPC:DMG-PEG=40:48.5:10:1.5. Solution of pre-mixed RNA is sodium citrate buffer, and a nitrogen phosphorus ratio for preparing LNP is 6:1.

Positive control group: using commercial lipids ALC-0315 and SM-102 to assemble a corresponding positive control LNP according to publicly available preparation method. Specific operation is: dissolving ALC-0315 or SM-102, DSPC, cholesterol, ALC-0159 or DMG-PEG2000 in anhydrous ethanol at concentrations of 5 mg/mL, 1.5 mg/mL, 3 mg/mL, and 1 mg/mL respectively. Mixing in a uniform molar ratio: ALC-0315: cholesterol:DSPC:ALC-0159=46.3:42.7:9.4:1.6 or SM-102: cholesterol:DSPC:DMG-PEG2000=50:38.5:10:1.5. At the same time, taking an appropriate amount of modRNA-Luc and dissolving in sodium citrate buffer (a volume of sodium citrate buffer is three times the total volume of the lipid mixture, pH=4.0). Then, buffer containing mRNA is rapidly mixed with a lipid mixture solution, incubated at room temperature for 15 minutes to assemble LNP, dialyzed in PBS using a dialysis bag (MWCO=14000 MW) for 1 hour, performing tail vein injection (each injection containing 5 g modRNA Luc of LNP). A ratio of nitrogen to phosphorus between ALC-0315, SM-102 and mRNA is 6:1.

### s2: Analysis of In Vivo Imaging Results

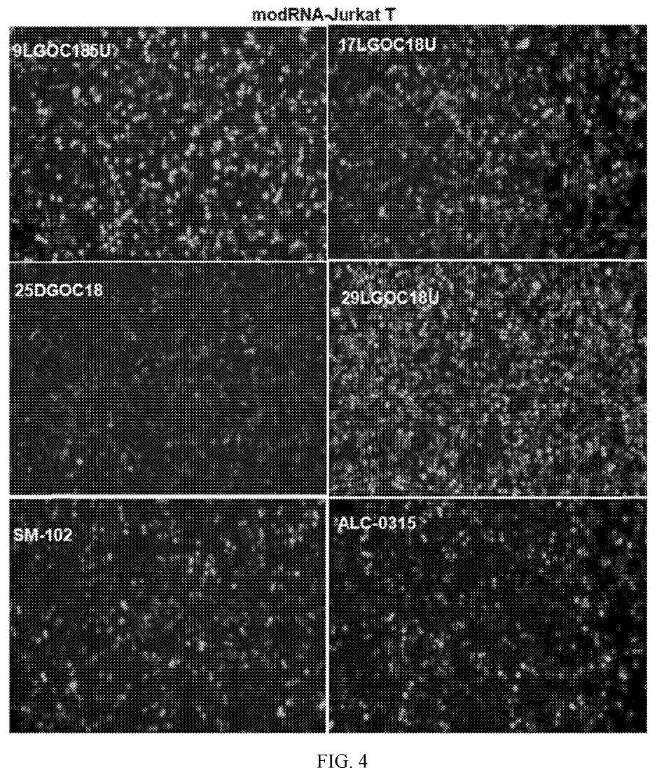

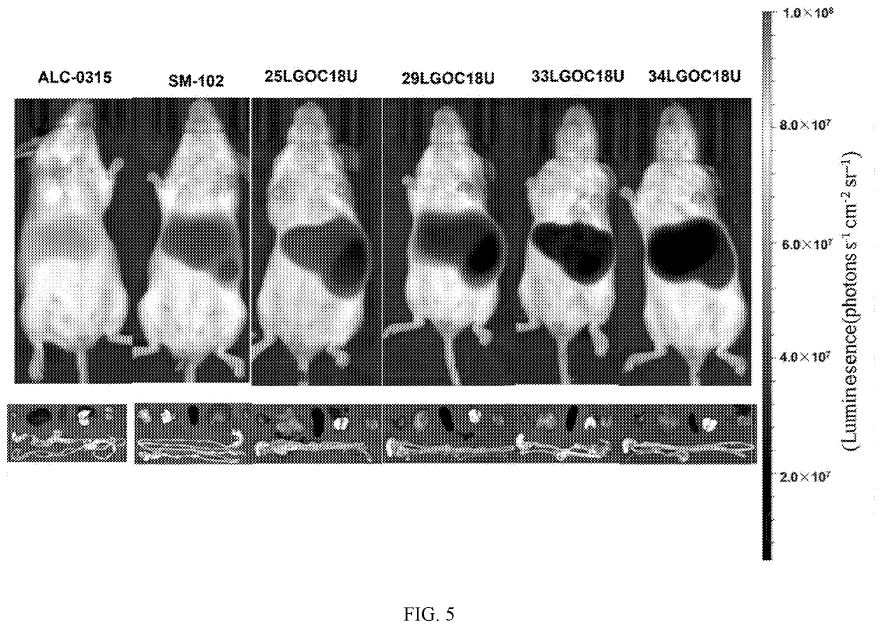

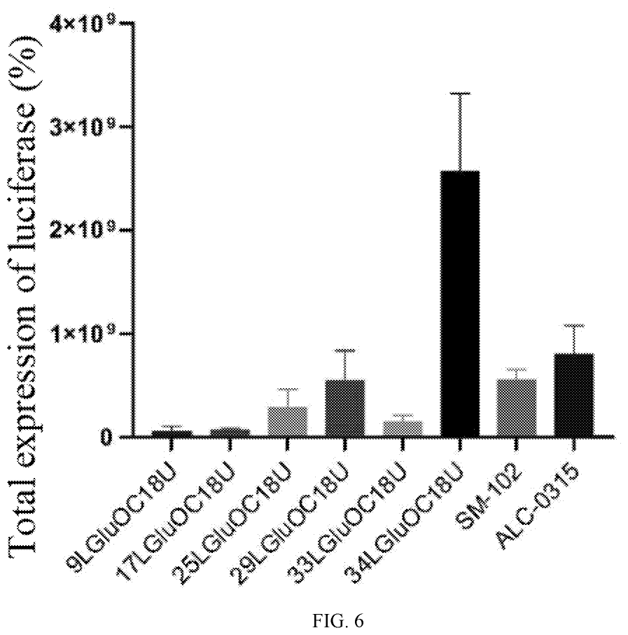

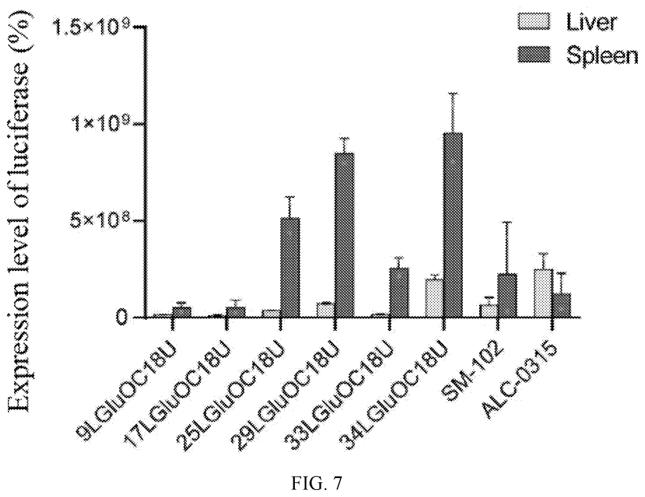

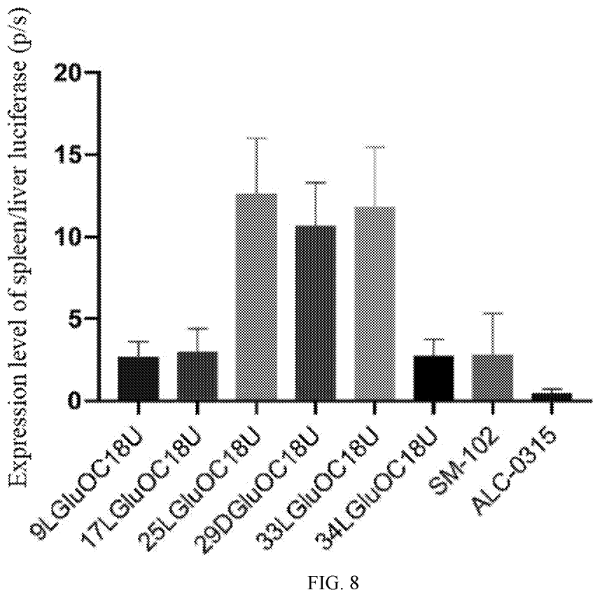

After 6 hours of tail vein injection, IVIS results shows (as shown in FIG. 5, FIG. 6, FIG. 7, FIG. 8) that a expression intensity of ionizable lipids 29GluOC18U and 34GluOC18U of the present disclosure is superior to commercial lipids SM-102 and ALC-0315, and have excellent organ targeting ability, which can accurately target the spleen.

According to literature reports, a vast majority of ionizable lipid loaded nucleic acids are expressed in multiple organs throughout a body after intravenous injection and exhibit liver tropism, while the ionizable lipids of the present disclosure can accurately target the spleen. The spleen is the largest immune organ in the body, and LNP targeting the spleen can bring more significant immune therapeutic effects in nucleic acid therapy applications.

The above is the preferred embodiment of the present disclosure. It should be pointed out that for ordinary technical personnel in this field, several improvements and embellishments can be made without departing from the principles of the present disclosure. These improvements and embellishments are also considered to be within the scope of protection of the present disclosure.

What is claimed is:

1. An amino acid backbone ionizable lipid, wherein is a compound, a pharmaceutically acceptable salt thereof, a stereoisomer thereof, or a tautomer thereof represented by formula (A) or formula (B); 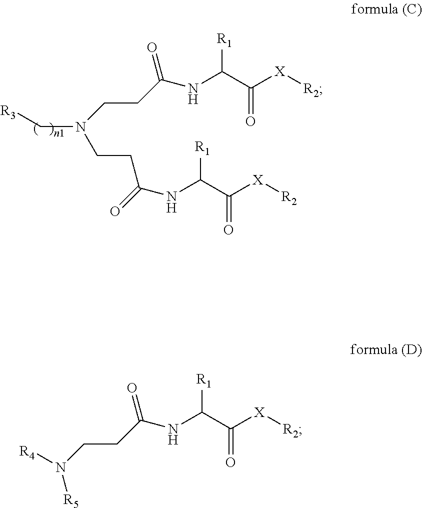
wherein R₁ is selected from 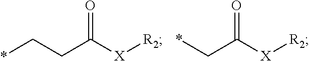
C2-C10 alkyl; C2-C10 heteroalkylene; arylalkyl or non-existence;
R₂ is selected from C3-C24 linear alkyl; C4-C24 linear alkenyl; 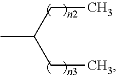
wherein n1=2-3, n2=3-24, n3=3-24;
X is O or NH;
R₃ is selected from 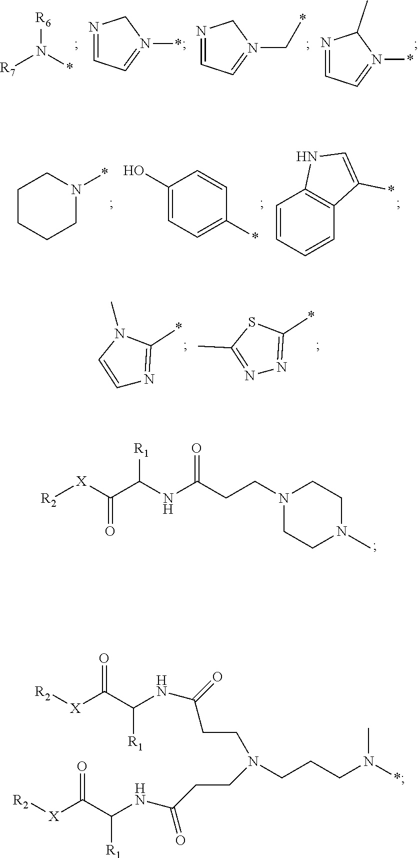
C1-C10 hydroxyalkyl; wherein R₆ is selected from C1-C4 alkyl, R₇ is selected from C1-C4 hydroxyalkyl;
wherein R₄ is selected from C1-C4 hydroxyalkyl; R₅ is C1-C4 alkyl, C1-C4 hydroxyalkyl, 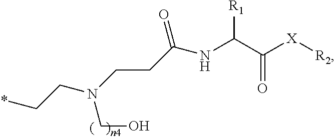
wherein n4=2-3;
wherein “*” is a connecting site.

2. The amino acid backbone ionizable lipid according to claim 1, a structure of the amino acid backbone ionizes lipid is represented by any one of formula (1) to formula (104):

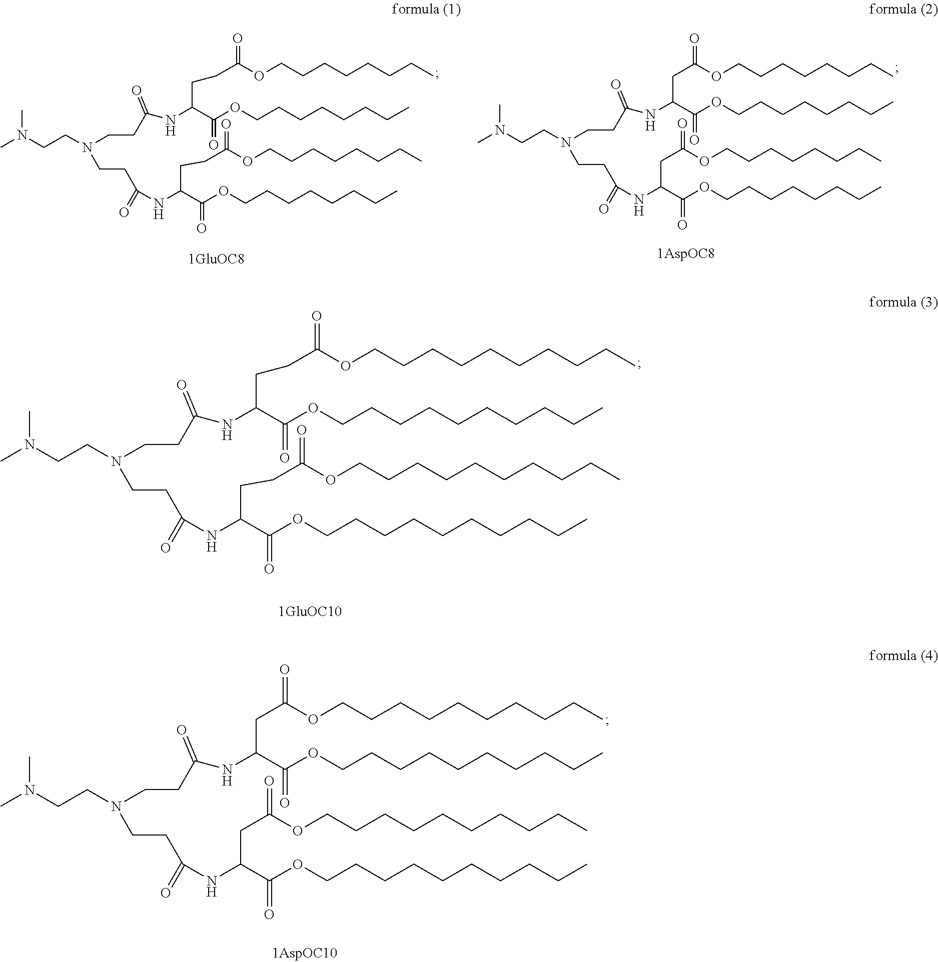

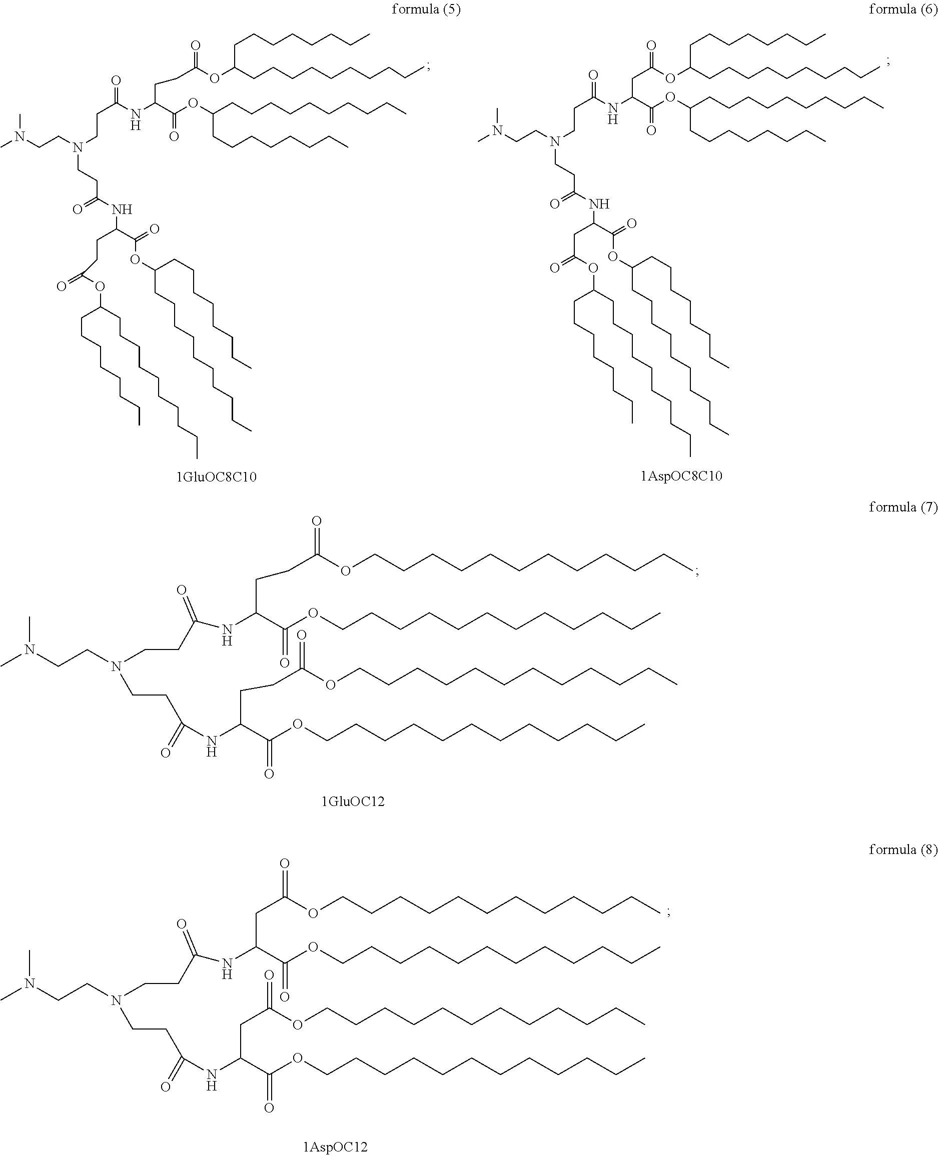

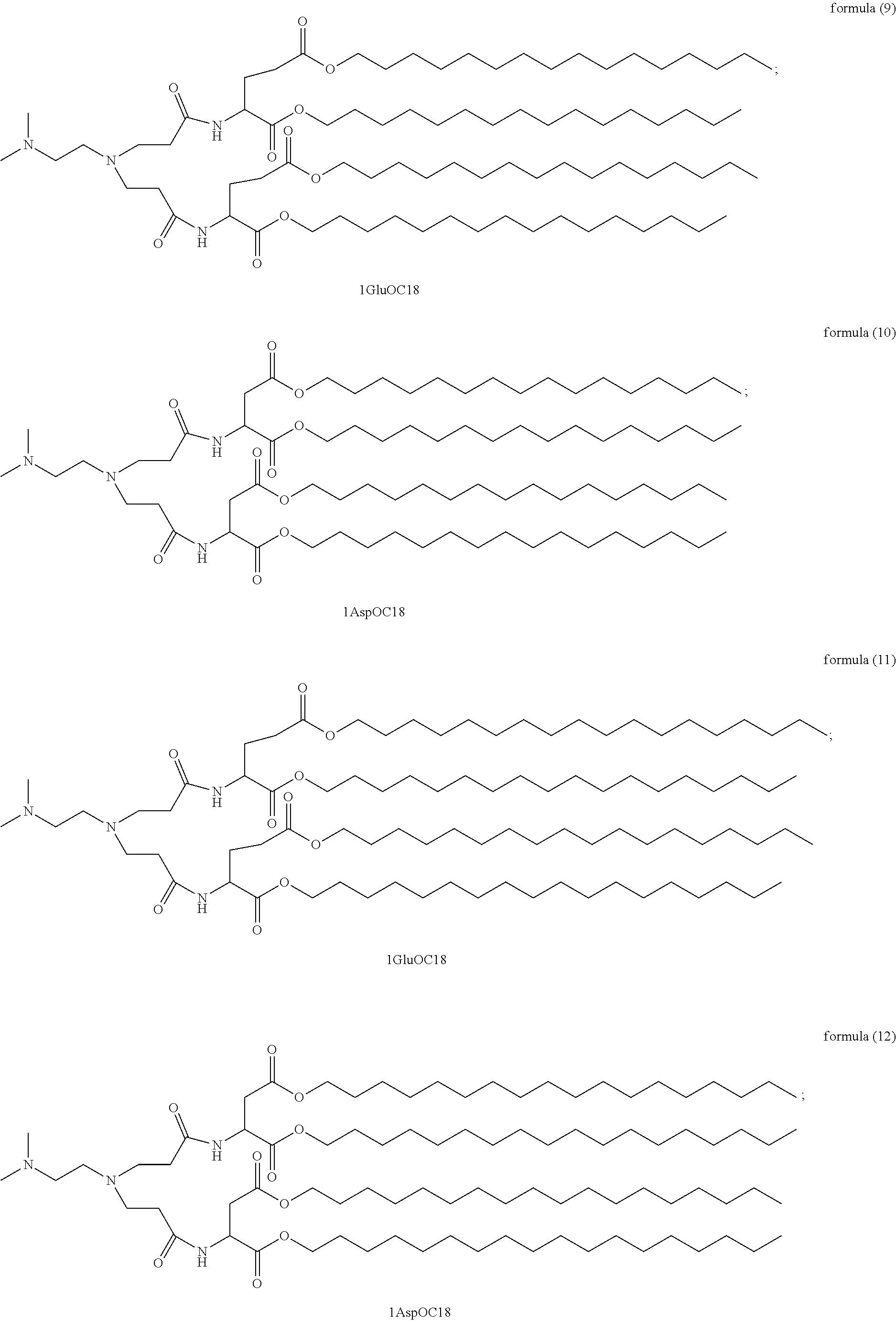

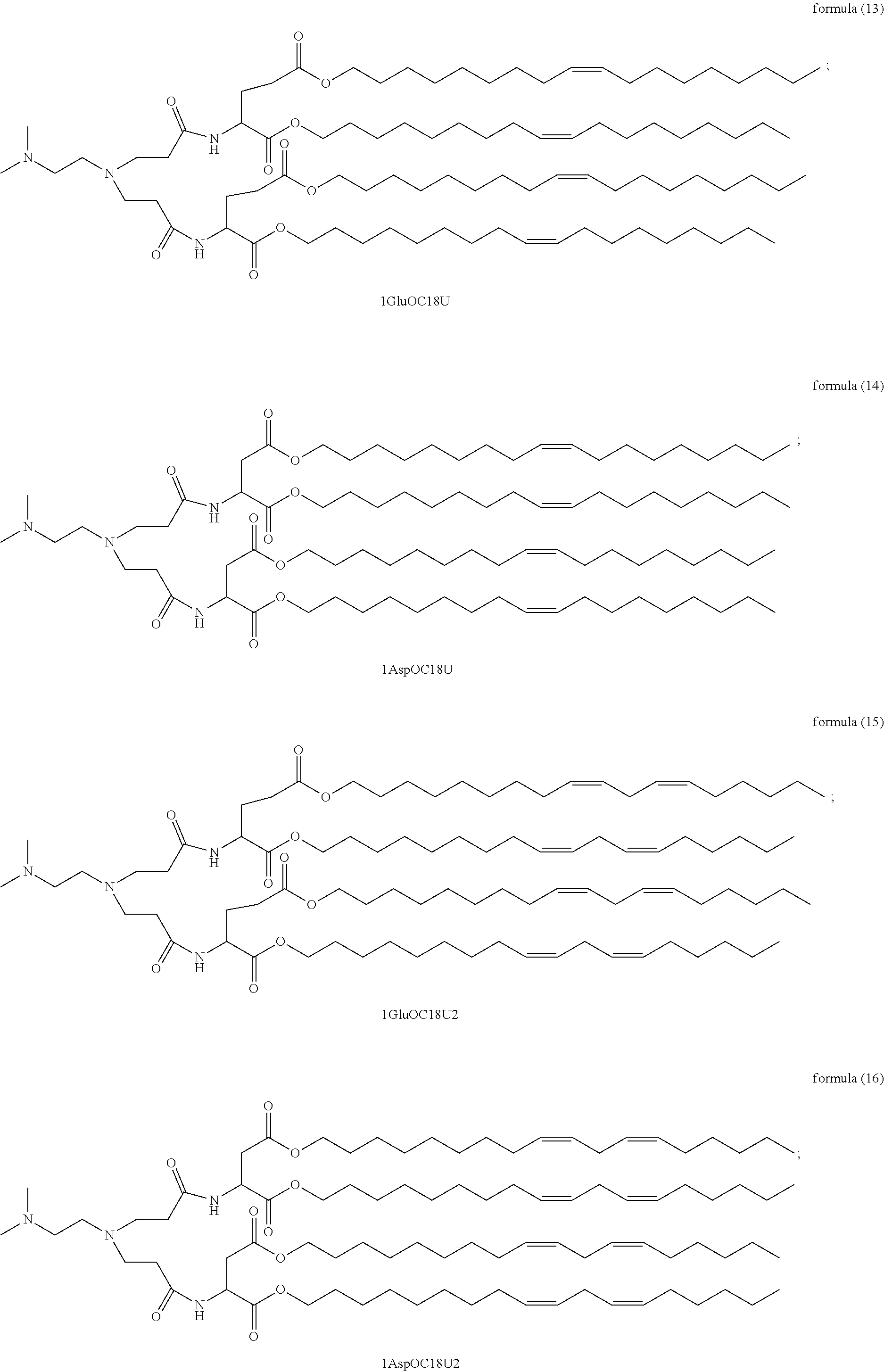

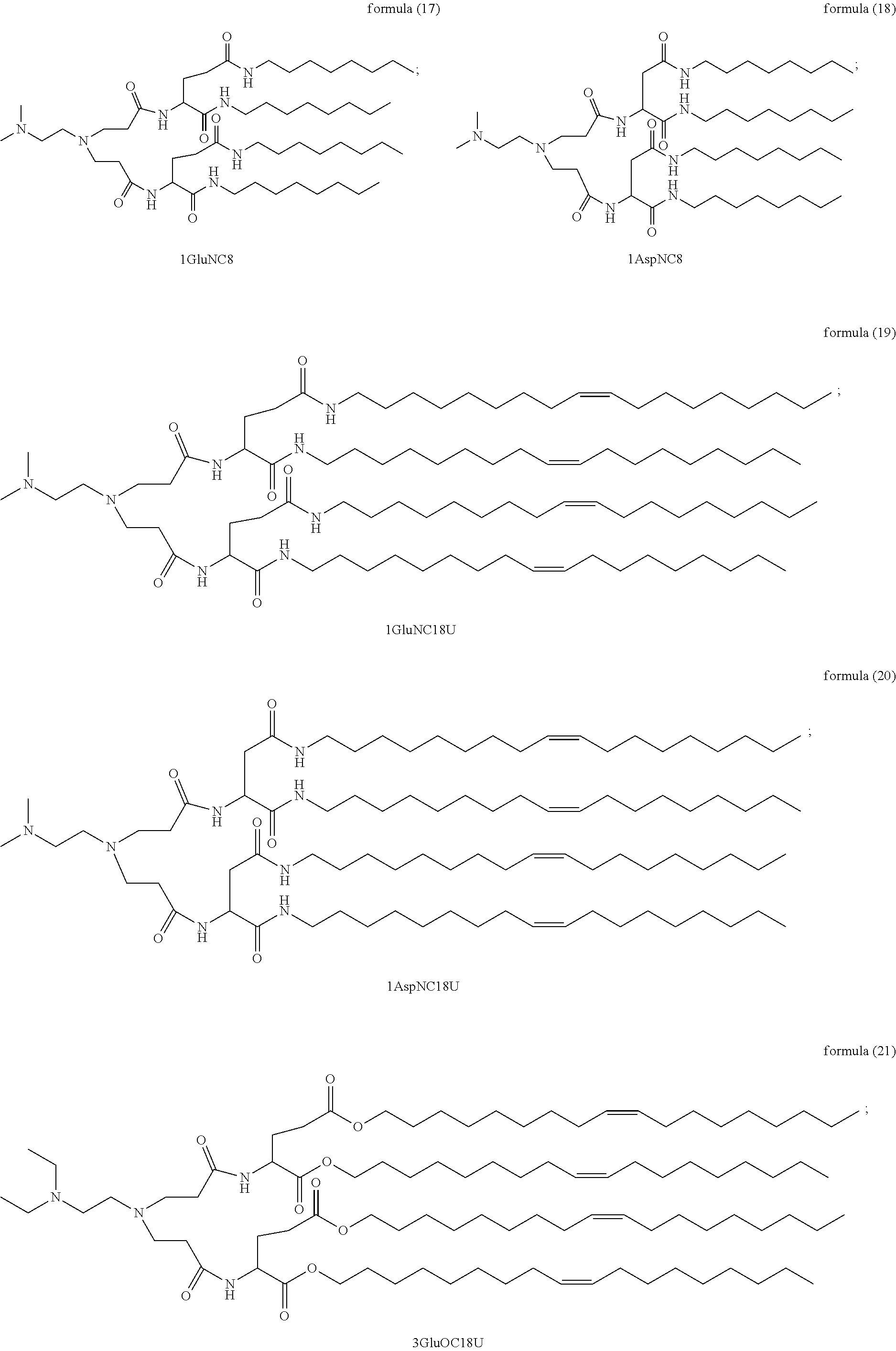

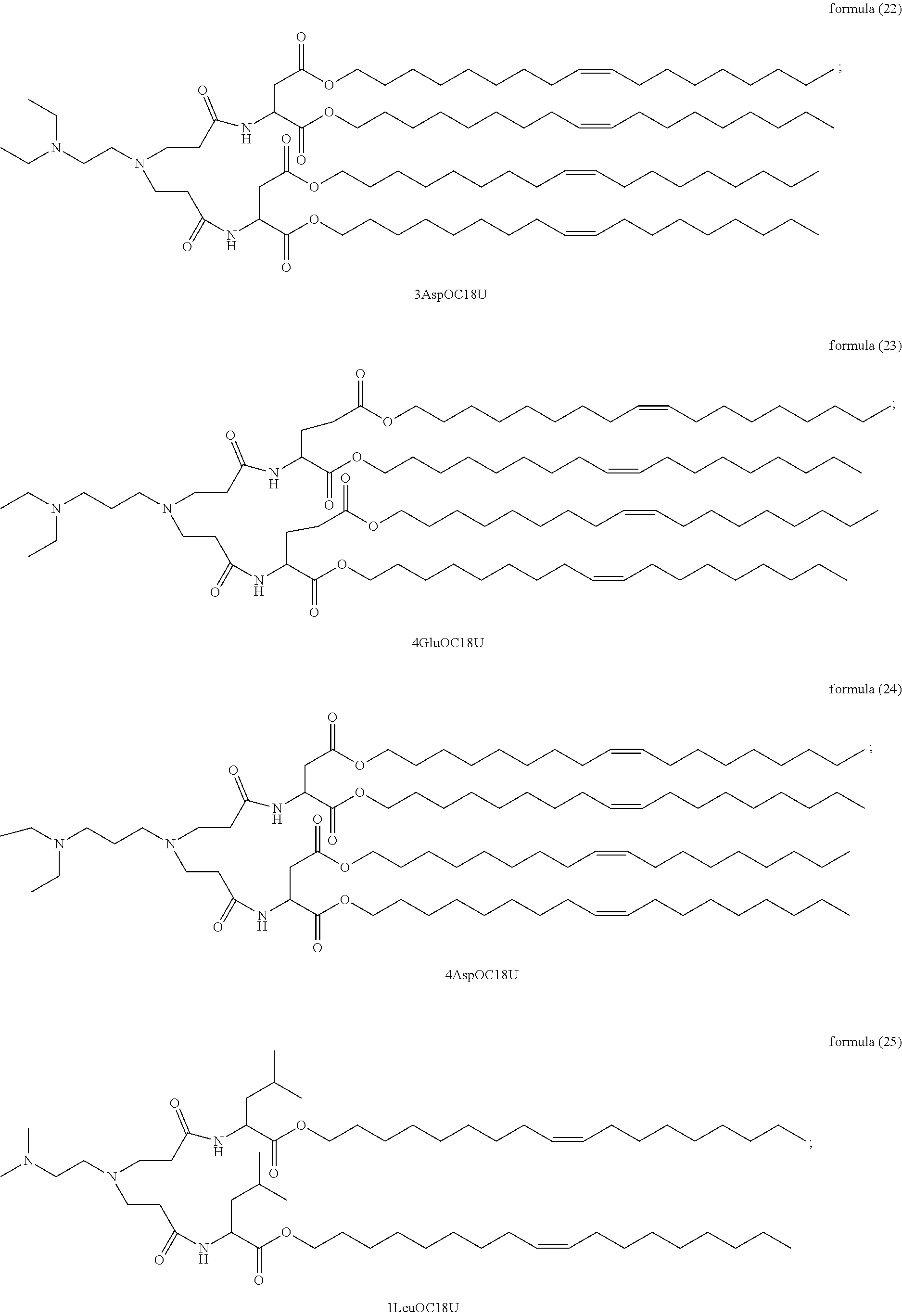

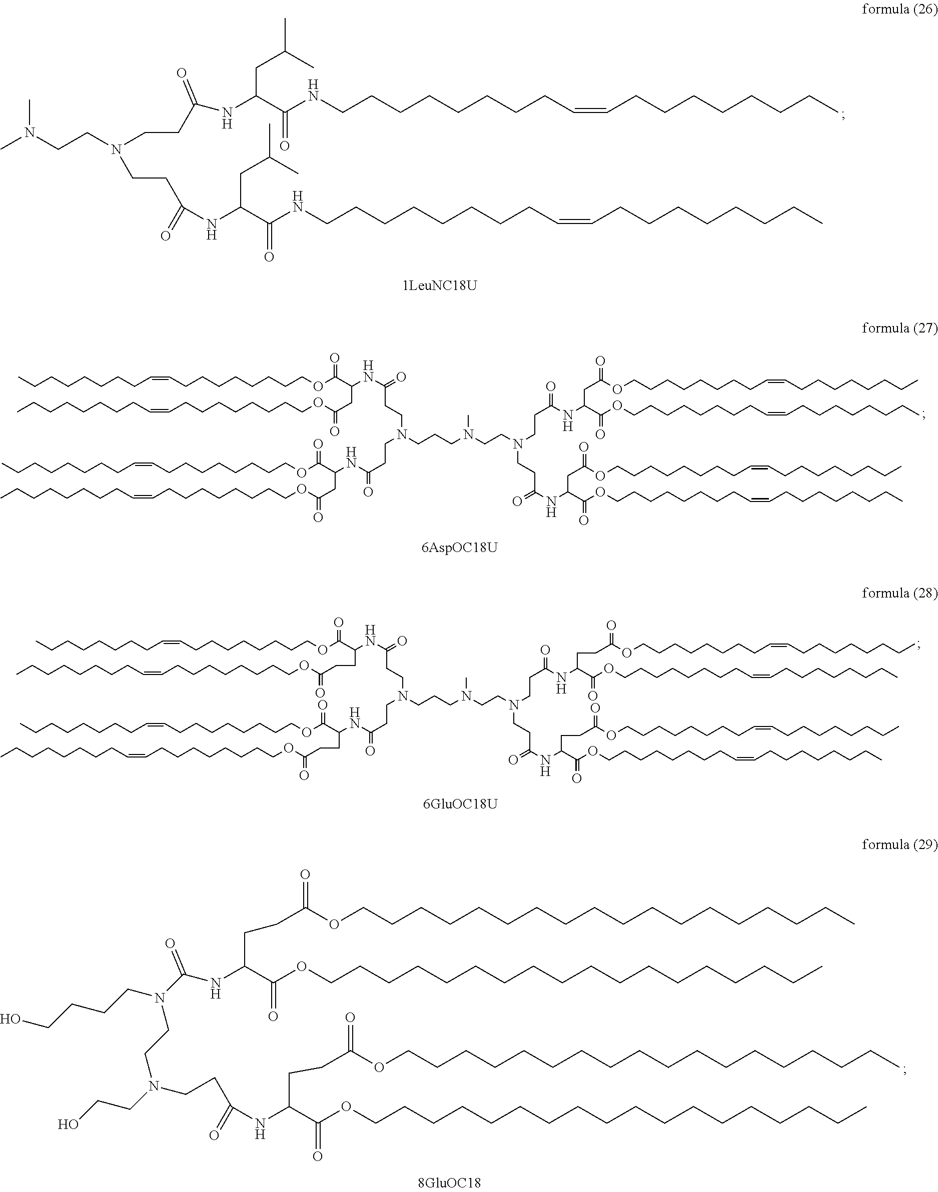

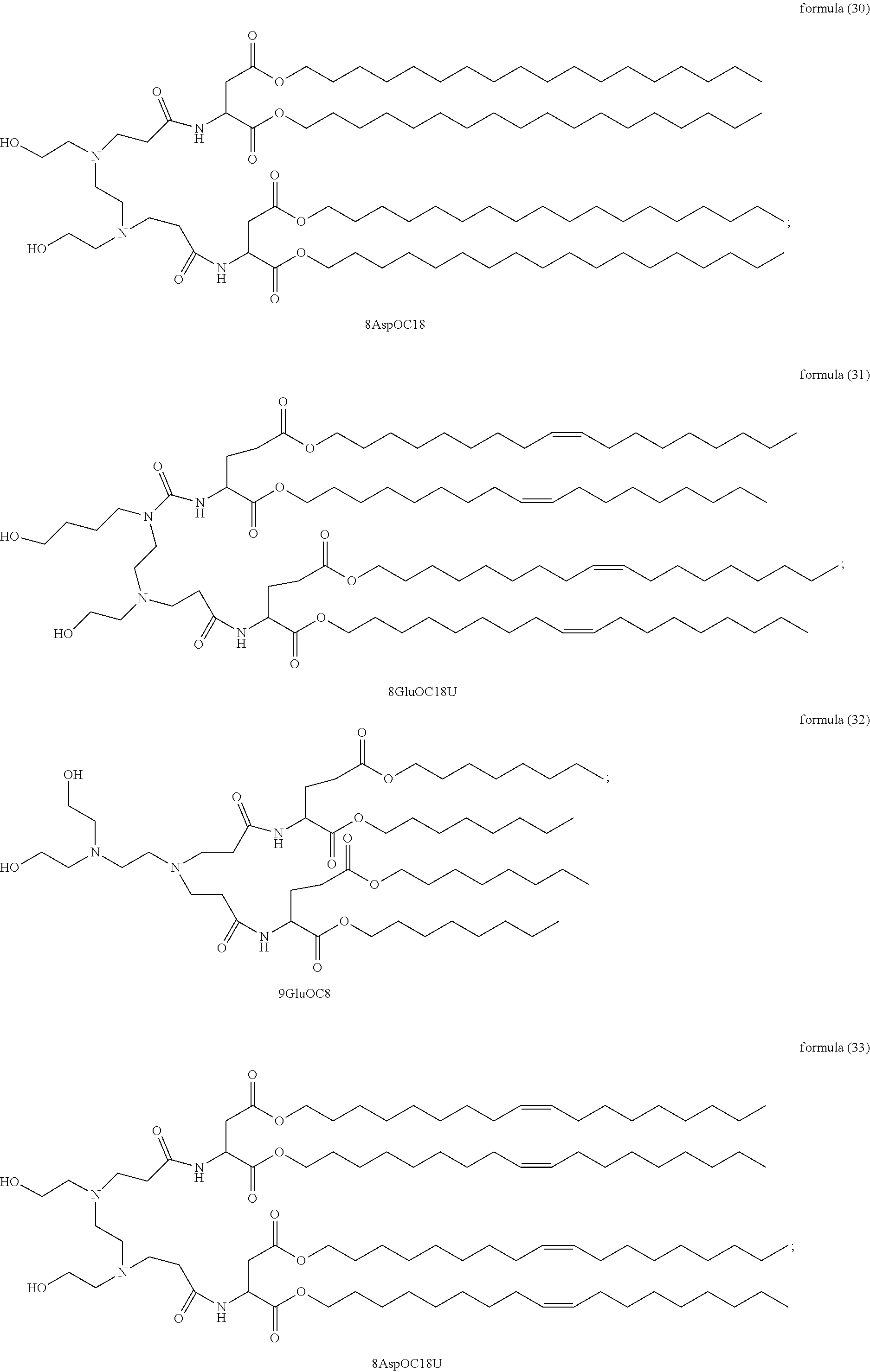

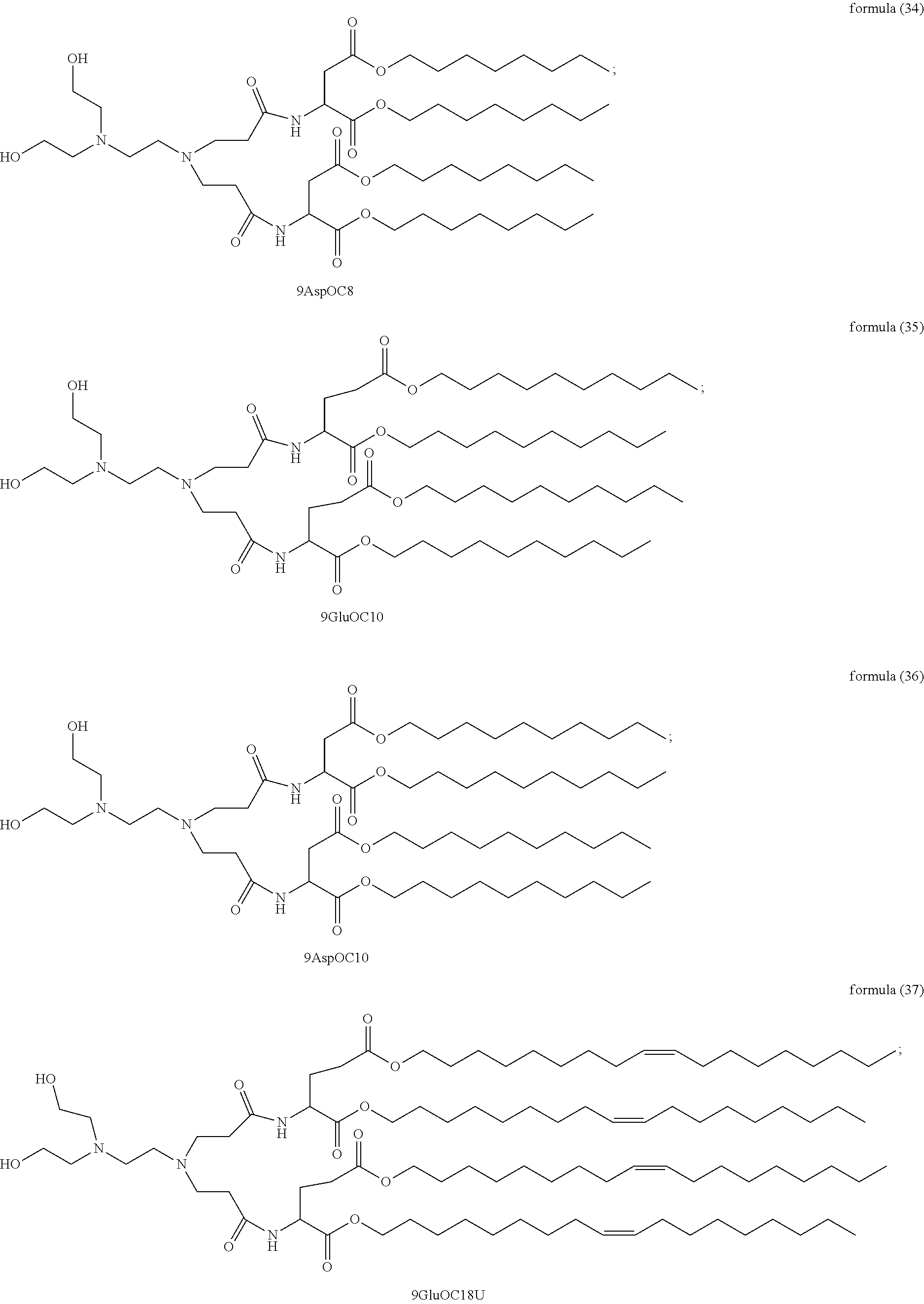 [SMILES: CCCCCCCCOC(=O)CC(NC(=O)CCN(CCC(=O)NC(CC(=O)OCCCCCCCC)C(=O)OCCCCCCCC)CCN(CCO)CCO)C(=O)OCCCCCCCC ; CCCCCCCCCCOC(=O)CCC(NC(=O)CCN(CCC(=O)NC(CCC(=O)OCCCCCCCCCC)C(=O)OCCCCCCCCCC)CCN(CCO)CCO)C(=O)OCCCCCCCCCC ; CCCCCCCCCCOC(=O)CC(NC(=O)CCN(CCC(=O)NC(CC(=O)OCCCCCCCCCC)C(=O)OCCCCCCCCCC)CCN(CCO)CCO)C(=O)OCCCCCCCCCC ; CCCCCCCC/C=C\CCCCCCCCOC(=O)CCC(NC(=O)CCN(CCC(=O)NC(CCC(=O)OCCCCCCCC/C=C\CCCCCCCC)C(=O)OCCCCCCCC/C=C\CCCCCCCC)CCN(CCO)CCO)C(=O)OCCCCCCCC/C=C\CCCCCCCC]

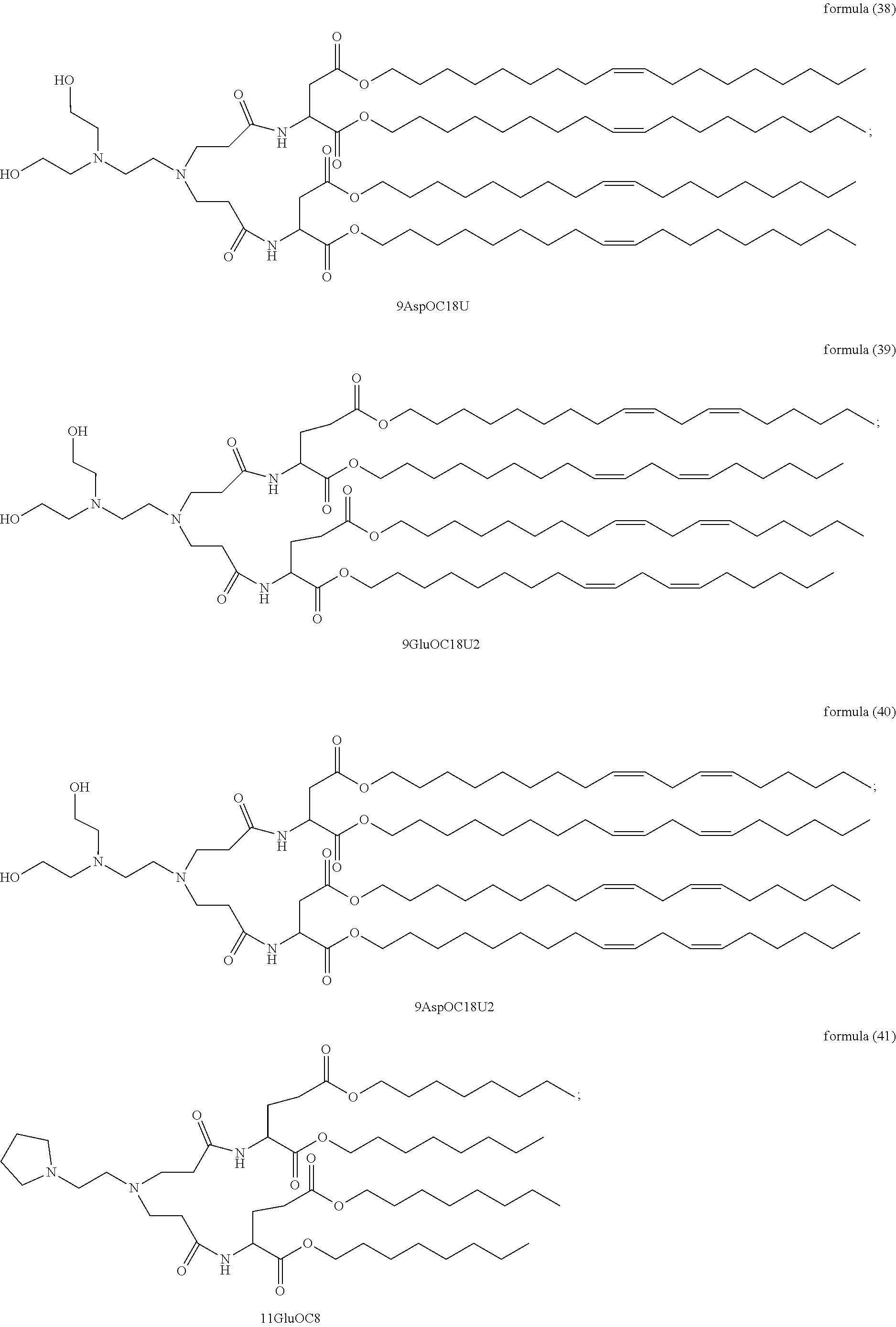

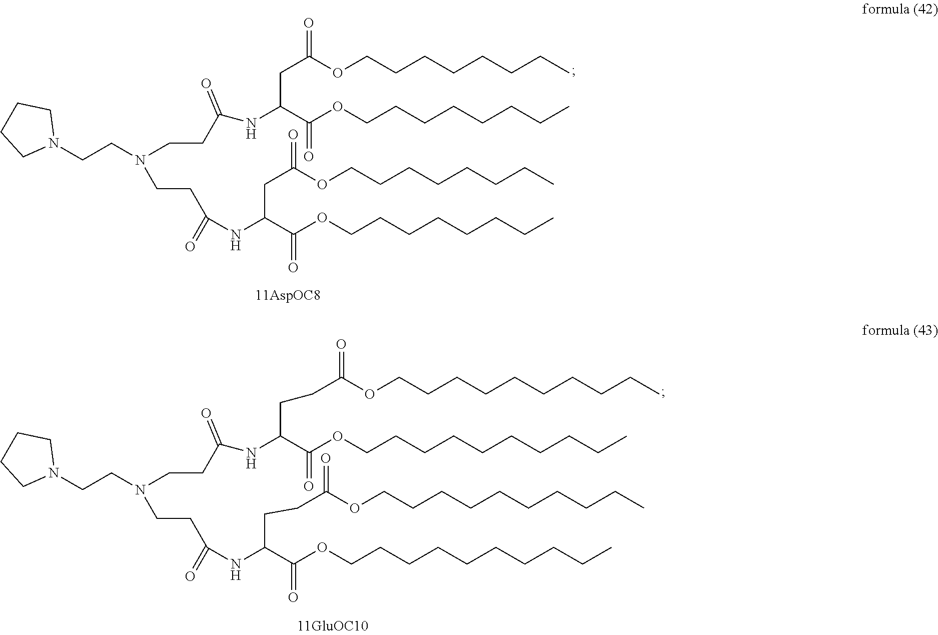 [SMILES: CCCCCCCCOC(=O)CC(NC(=O)CCN(CCC(=O)NC(CC(=O)OCCCCCCCC)C(=O)OCCCCCCCC)CCN1CCCC1)C(=O)OCCCCCCCC ; CCCCCCCCCCOC(=O)CCC(NC(=O)CCN(CCC(=O)NC(CCC(=O)OCCCCCCCCCC)C(=O)OCCCCCCCCCC)CCN1CCCC1)C(=O)OCCCCCCCCCC]

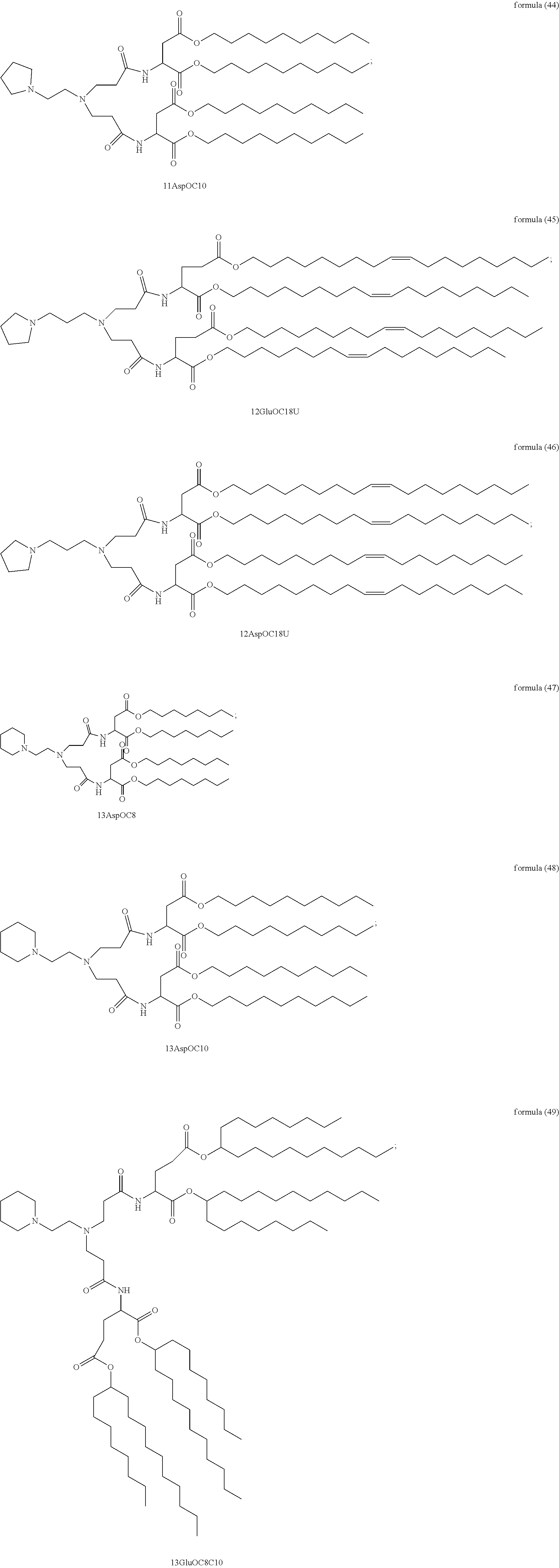

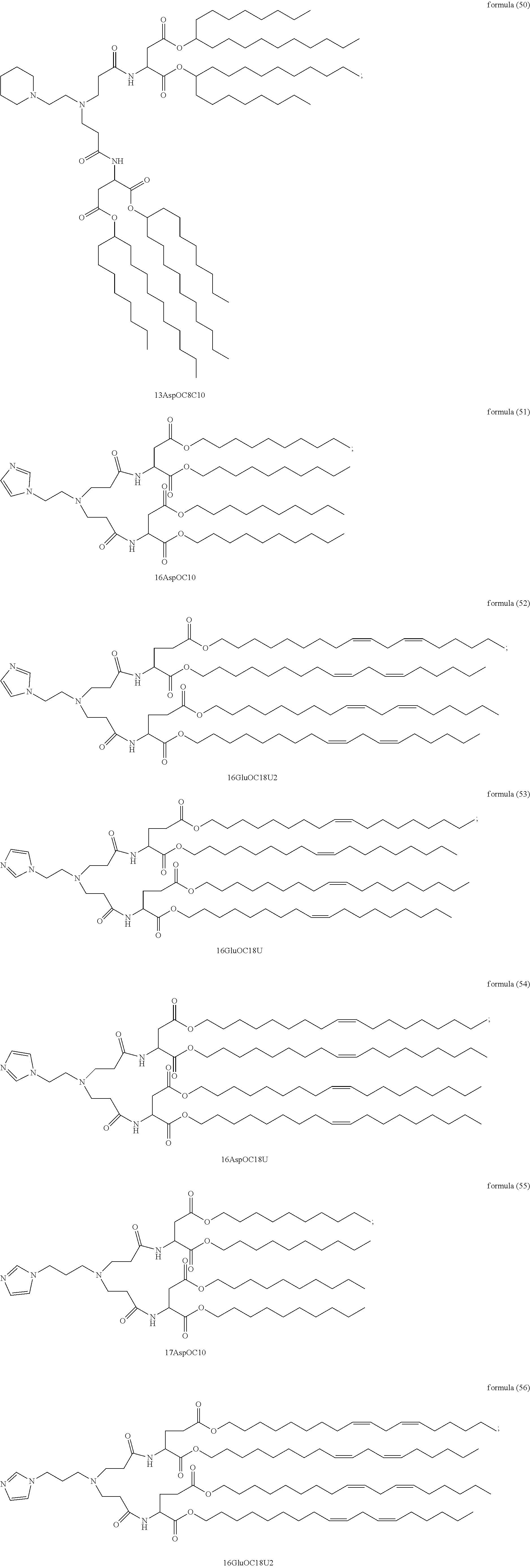

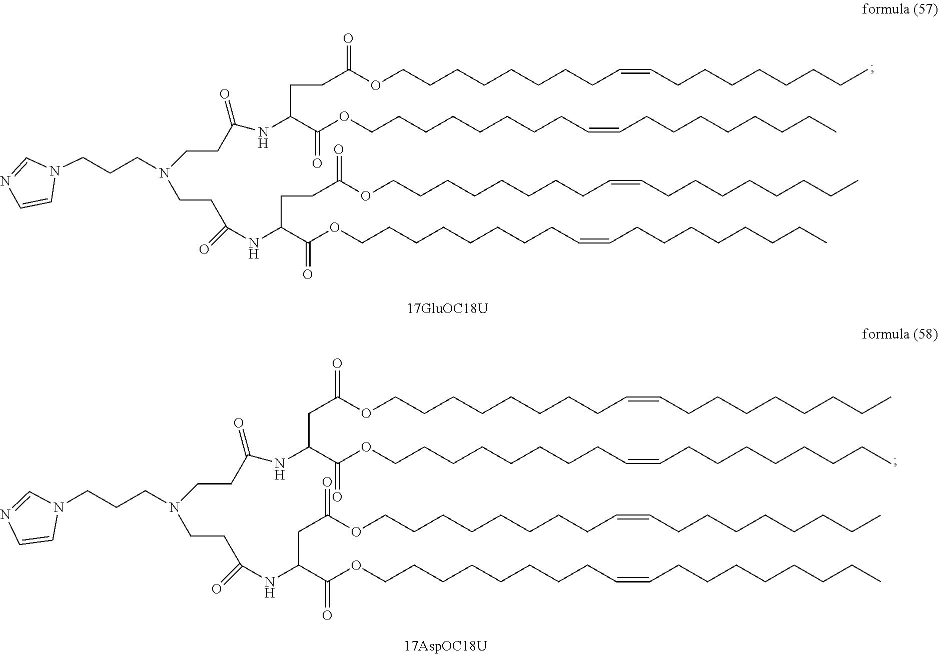 [SMILES: CCCCCCCC/C=C\CCCCCCCCOC(=O)CCC(NC(=O)CCN(CCCn1ccnc1)CCC(=O)NC(CCC(=O)OCCCCCCCC/C=C\CCCCCCCC)C(=O)OCCCCCCCC/C=C\CCCCCCCC)C(=O)OCCCCCCCC/C=C\CCCCCCCC ; CCCCCCCC/C=C\CCCCCCCCOC(=O)CC(NC(=O)CCN(CCCn1ccnc1)CCC(=O)NC(CC(=O)OCCCCCCCC/C=C\CCCCCCCC)C(=O)OCCCCCCCC/C=C\CCCCCCCC)C(=O)OCCCCCCCC/C=C\CCCCCCCC]

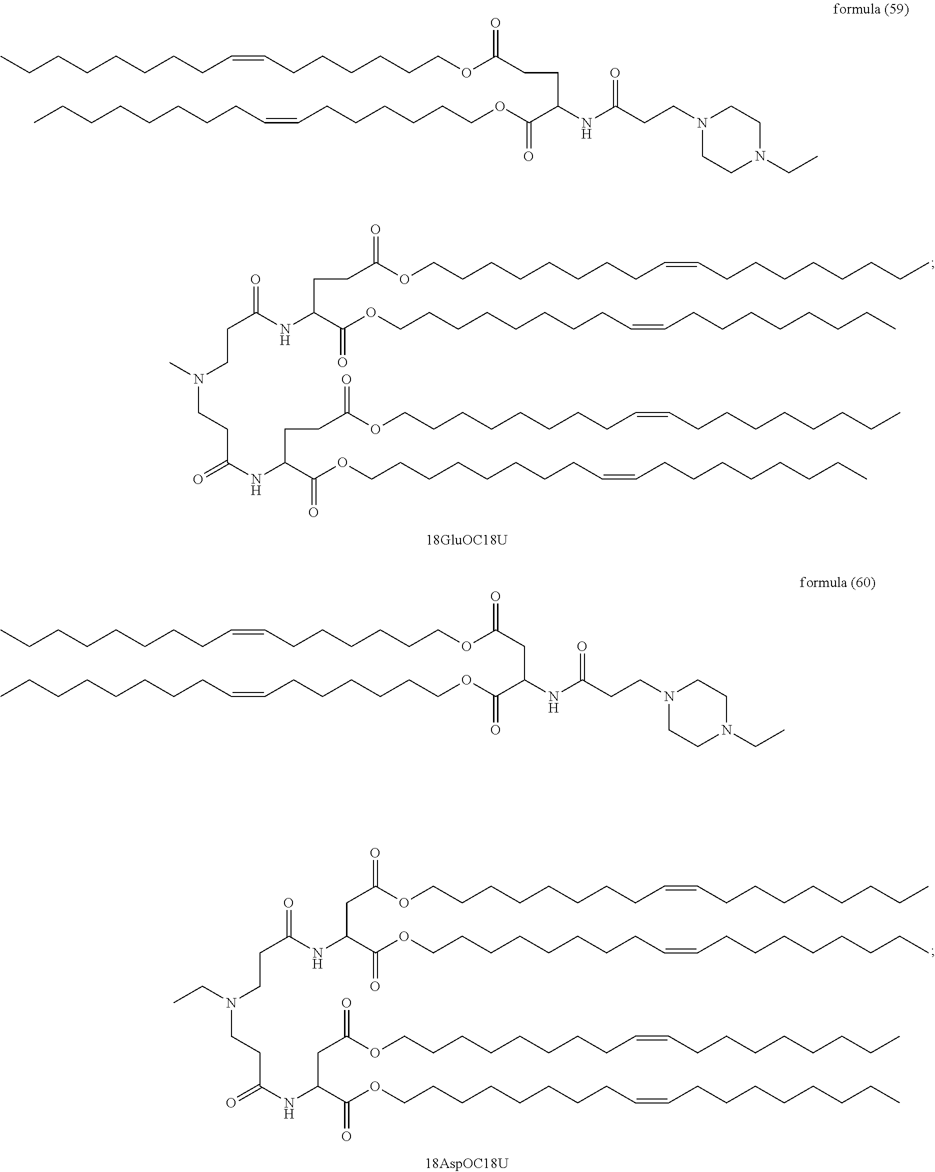

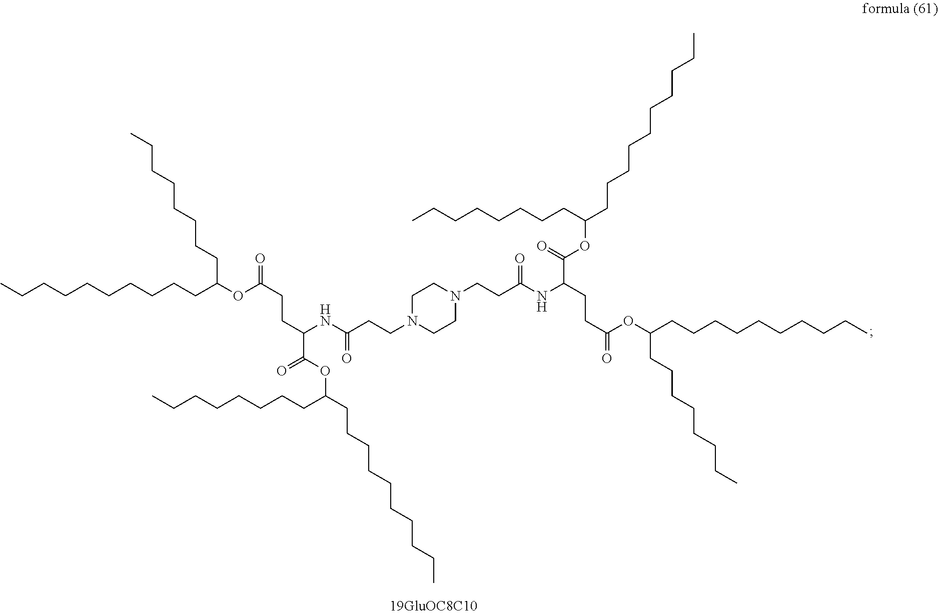 [SMILES: CCCCCCCCCCC(CCCCCCCC)OC(=O)CCC(NC(=O)CCN1CCN(CCC(=O)NC(CCC(=O)OC(CCCCCCCC)CCCCCCCCCC)C(=O)OC(CCCCCCCC)CCCCCCCCCC)CC1)C(=O)OC(CCCCCCCC)CCCCCCCCCC]

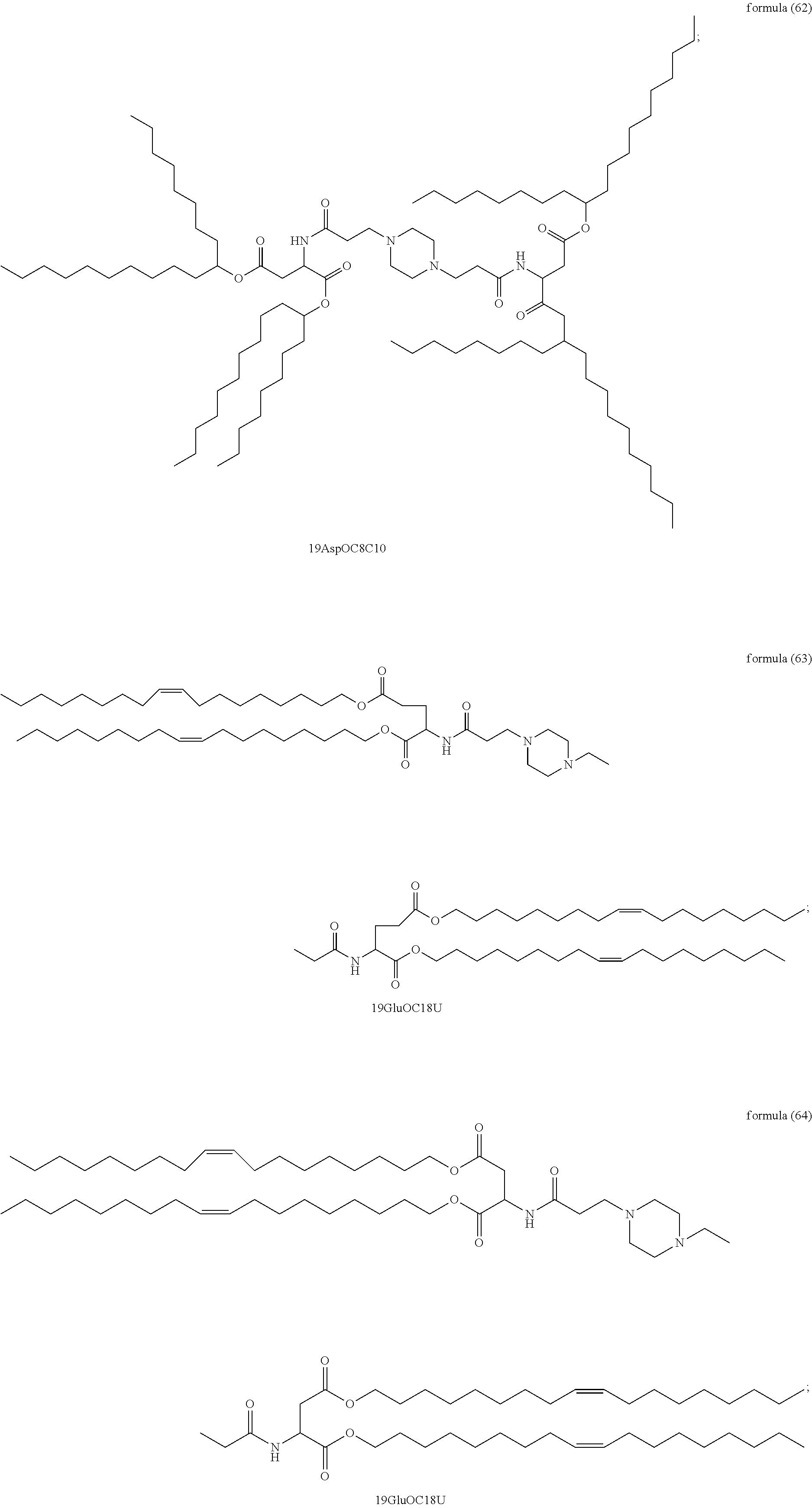

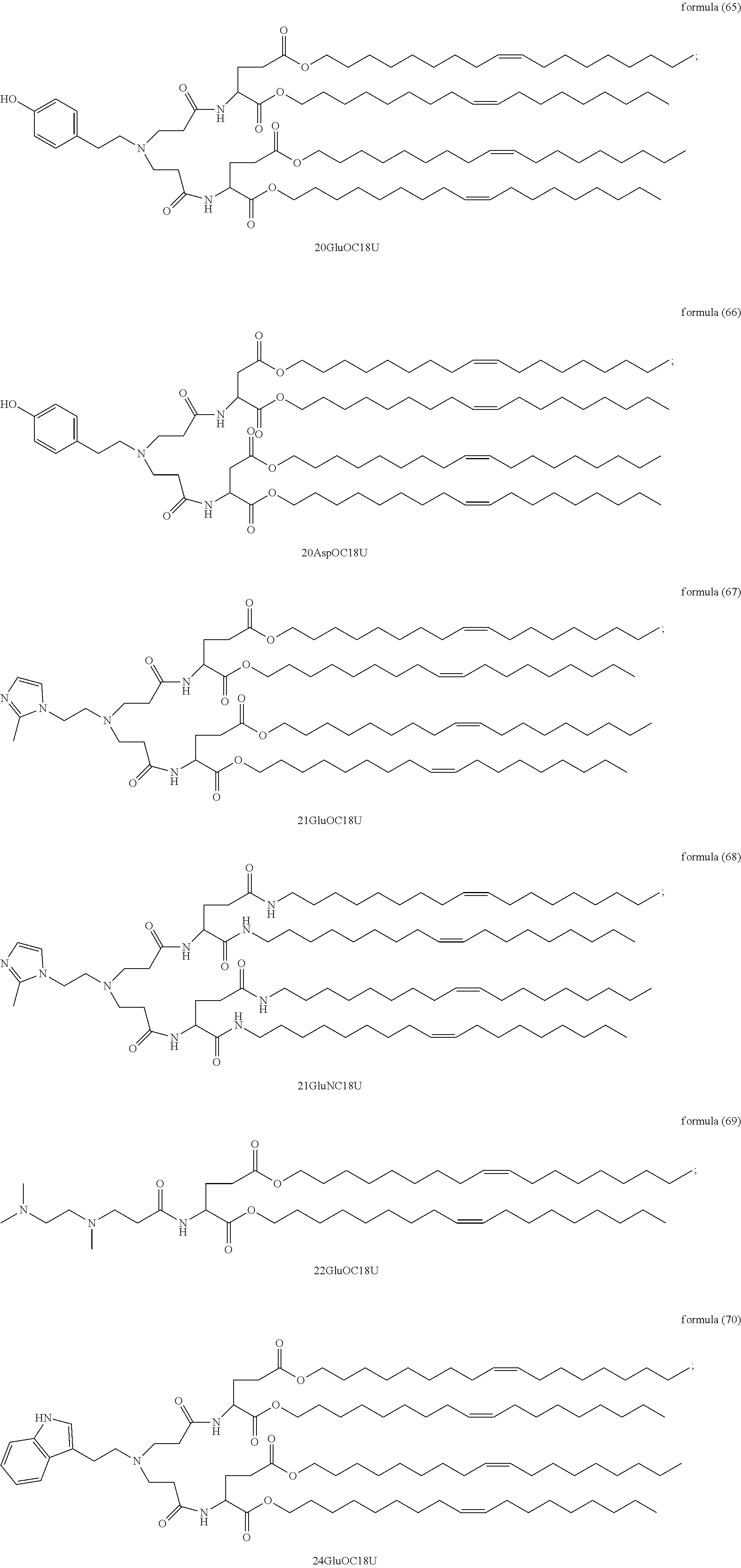

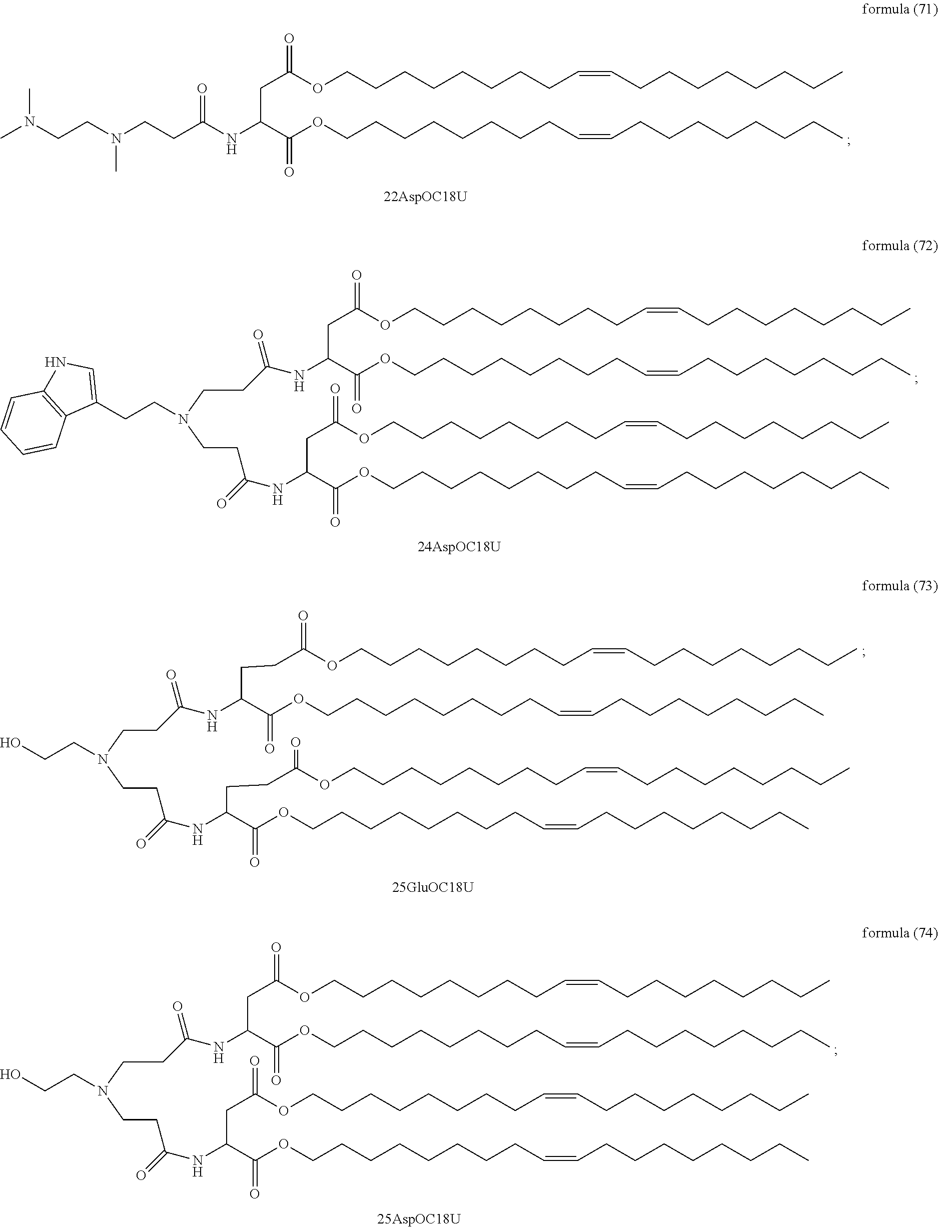

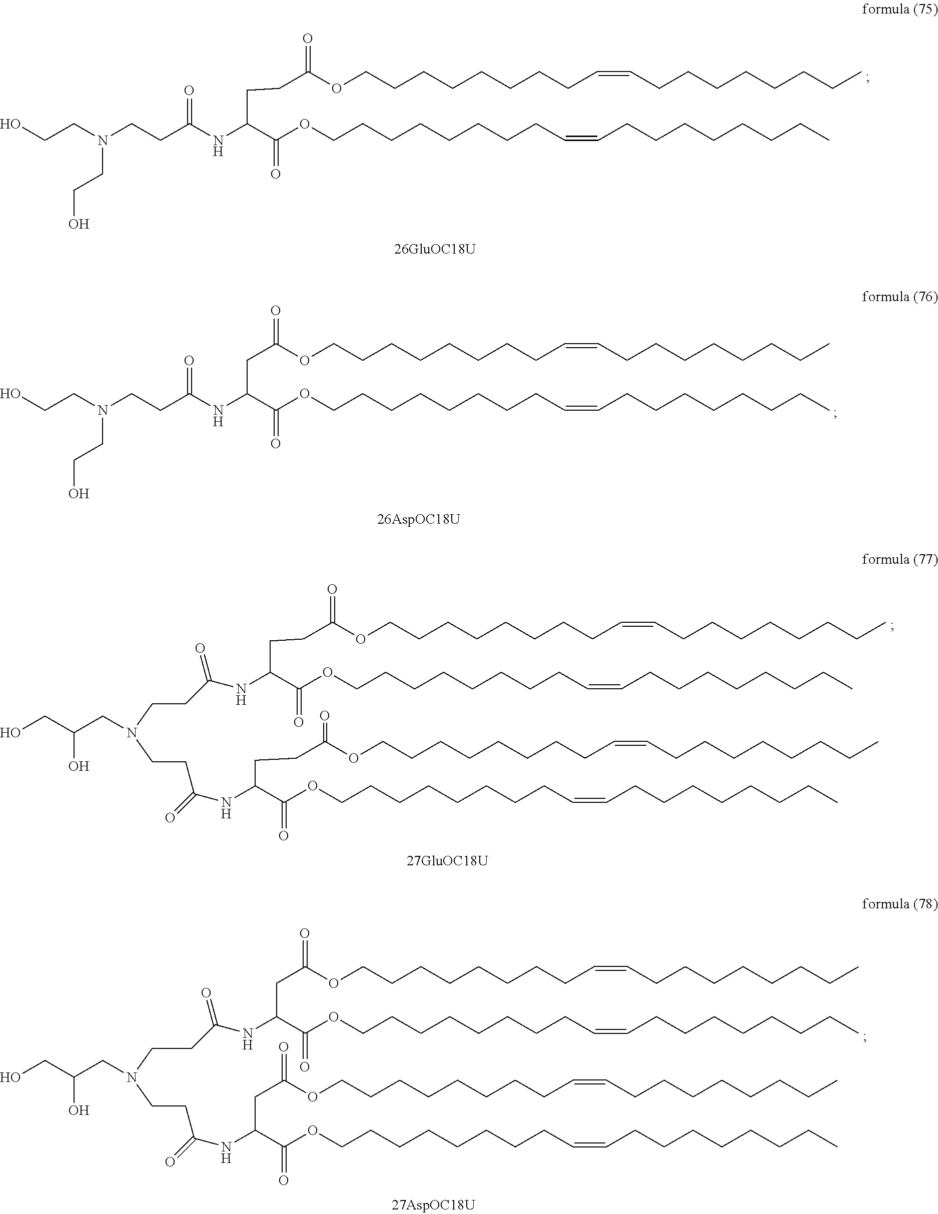 [SMILES: CCCCCCCC/C=C\CCCCCCCCOC(=O)CCC(NC(=O)CCN(CCO)CCO)C(=O)OCCCCCCCC/C=C\CCCCCCCC ; CCCCCCCC/C=C\CCCCCCCCOC(=O)CC(NC(=O)CCN(CCO)CCO)C(=O)OCCCCCCCC/C=C\CCCCCCCC ; CCCCCCCC/C=C\CCCCCCCCOC(=O)CCC(NC(=O)CCN(CCC(=O)NC(CCC(=O)OCCCCCCCC/C=C\CCCCCCCC)C(=O)OCCCCCCCC/C=C\CCCCCCCC)CC(O)CO)C(=O)OCCCCCCCC/C=C\CCCCCCCC ; CCCCCCCC/C=C\CCCCCCCCOC(=O)CC(NC(=O)CCN(CCC(=O)NC(CC(=O)OCCCCCCCC/C=C\CCCCCCCC)C(=O)OCCCCCCCC/C=C\CCCCCCCC)CC(O)CO)C(=O)OCCCCCCCC/C=C\CCCCCCCC]

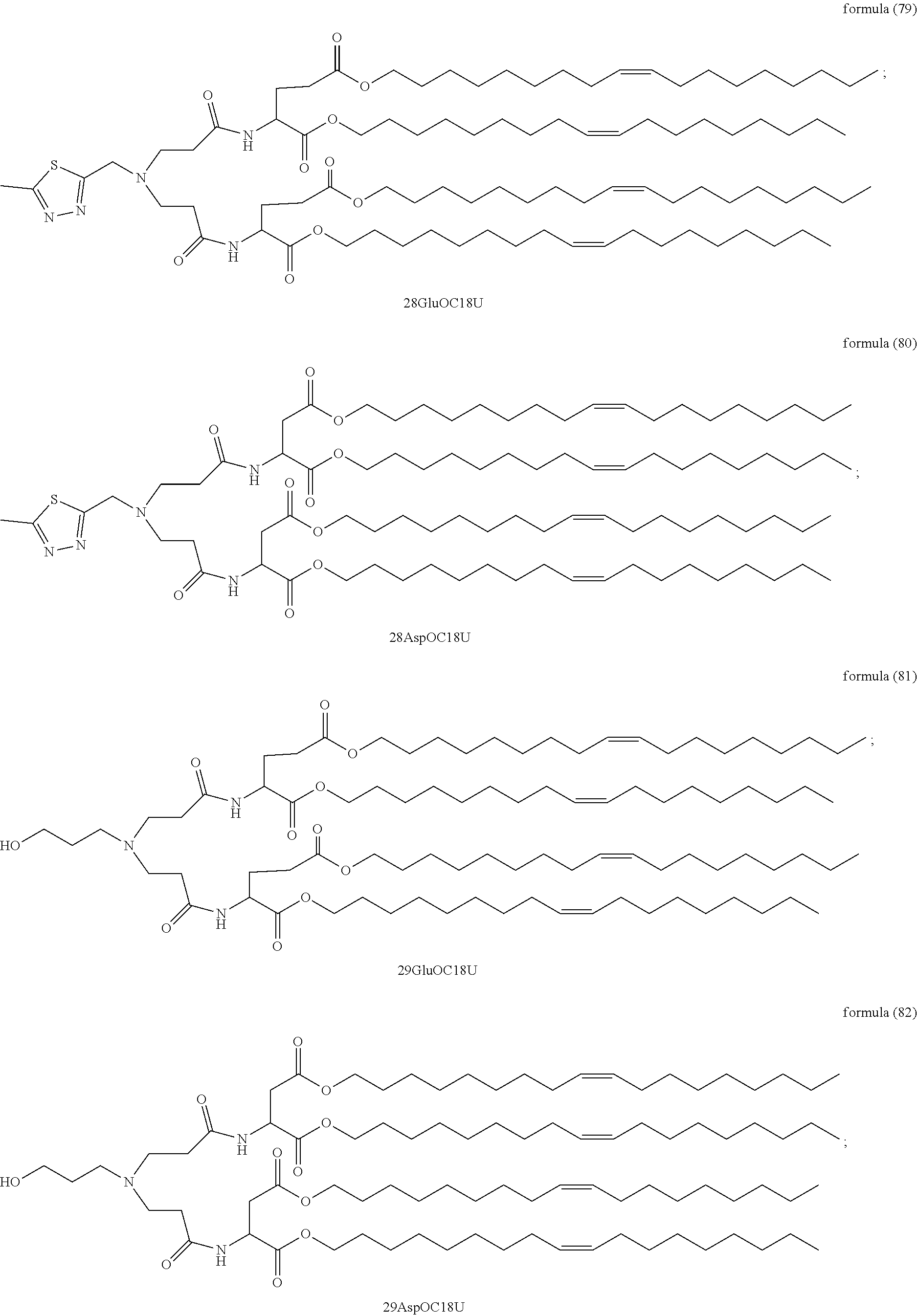

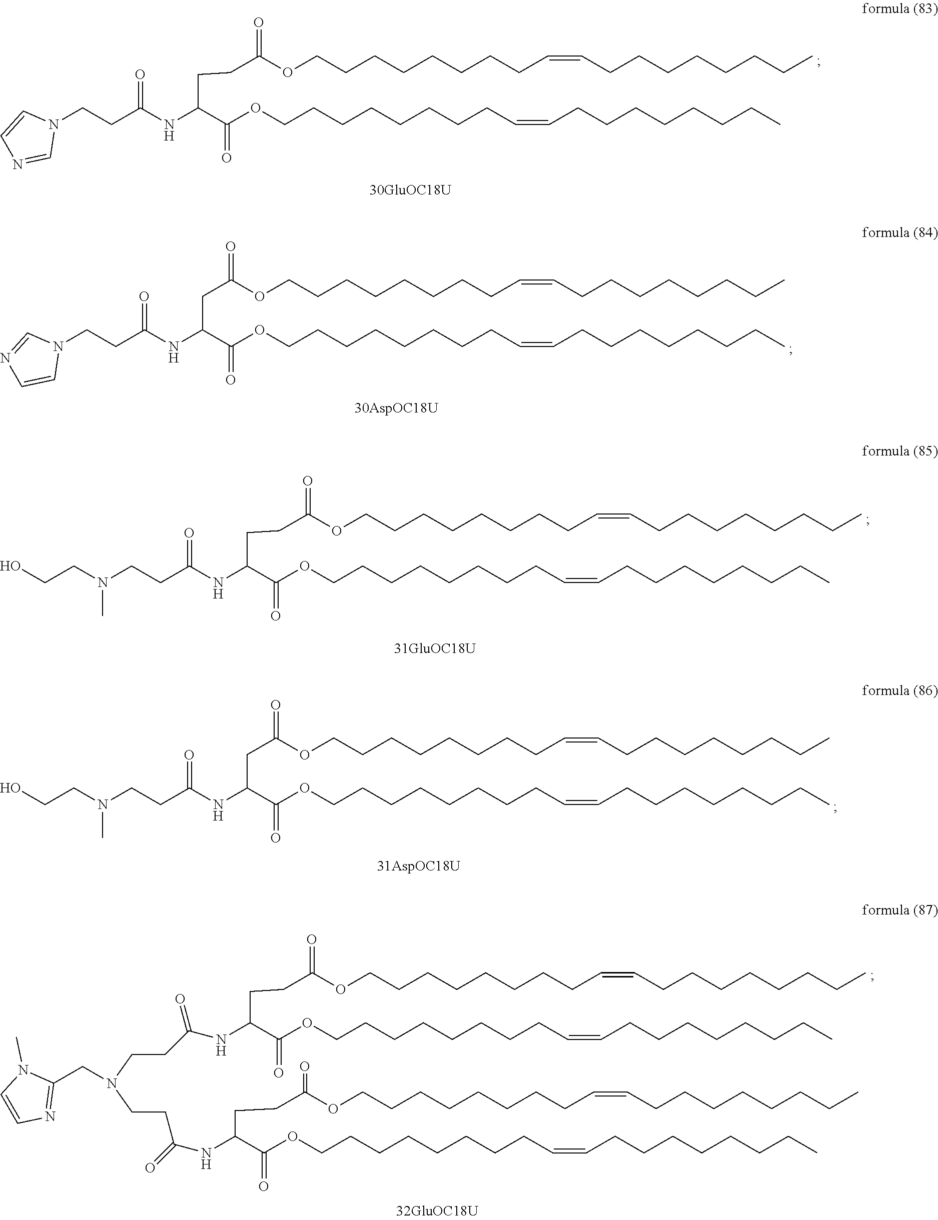 [SMILES: CCCCCCCC/C=C\CCCCCCCCOC(=O)CCC(NC(=O)CCn1ccnc1)C(=O)OCCCCCCCC/C=C\CCCCCCCC ; CCCCCCCC/C=C\CCCCCCCCOC(=O)CC(NC(=O)CCn1ccnc1)C(=O)OCCCCCCCC/C=C\CCCCCCCC ; CCCCCCCC/C=C\CCCCCCCCOC(=O)CCC(NC(=O)CCN(C)CCO)C(=O)OCCCCCCCC/C=C\CCCCCCCC ; CCCCCCCC/C=C\CCCCCCCCOC(=O)CC(NC(=O)CCN(C)CCO)C(=O)OCCCCCCCC/C=C\CCCCCCCC ; CCCCCCCC/C=C\CCCCCCCCOC(=O)CCC(NC(=O)CCN(CCC(=O)NC(CCC(=O)OCCCCCCCC/C=C\CCCCCCCC)C(=O)OCCCCCCCC/C=C\CCCCCCCC)Cc1nccn1C)C(=O)OCCCCCCCC/C=C\CCCCCCCC]

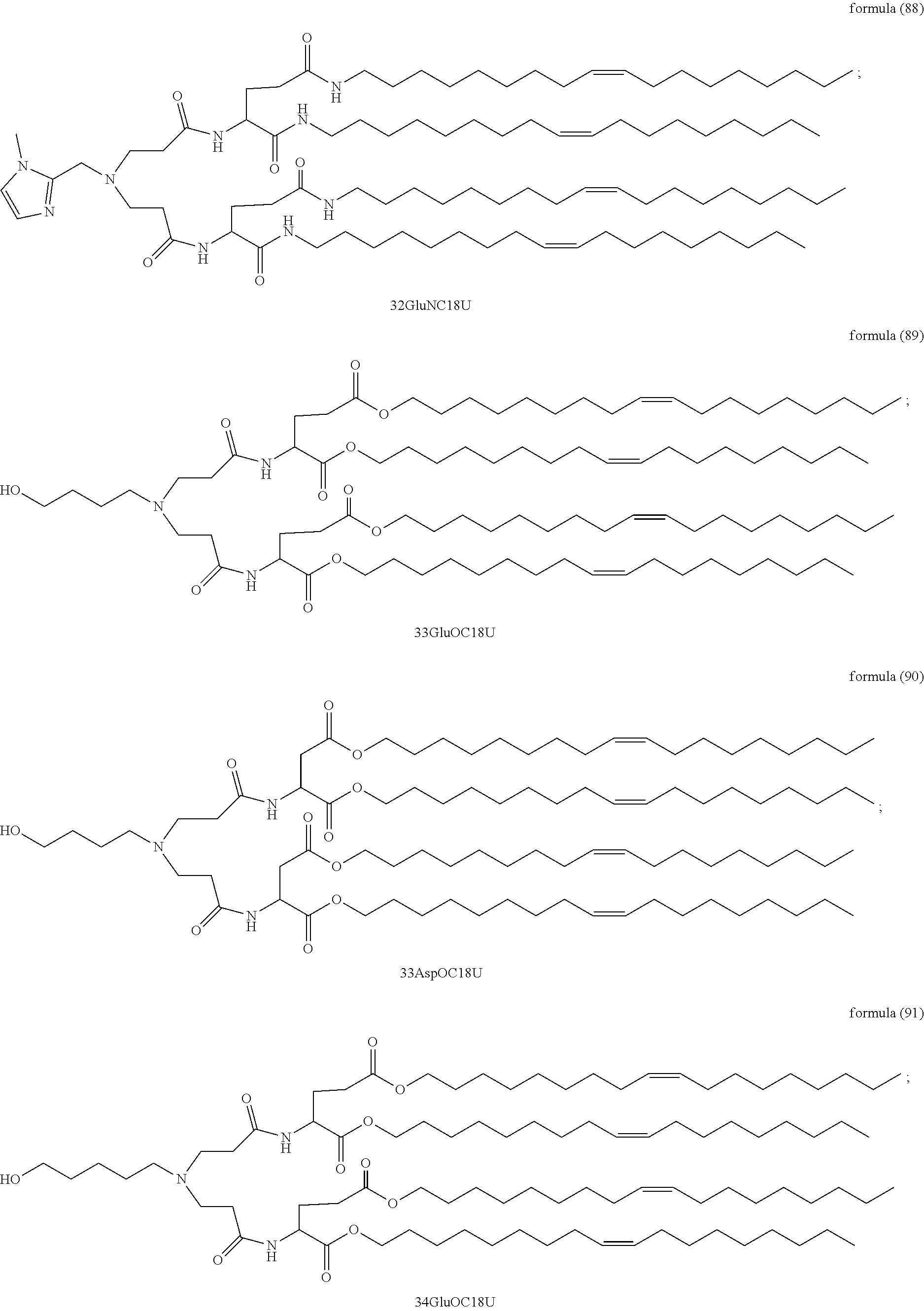

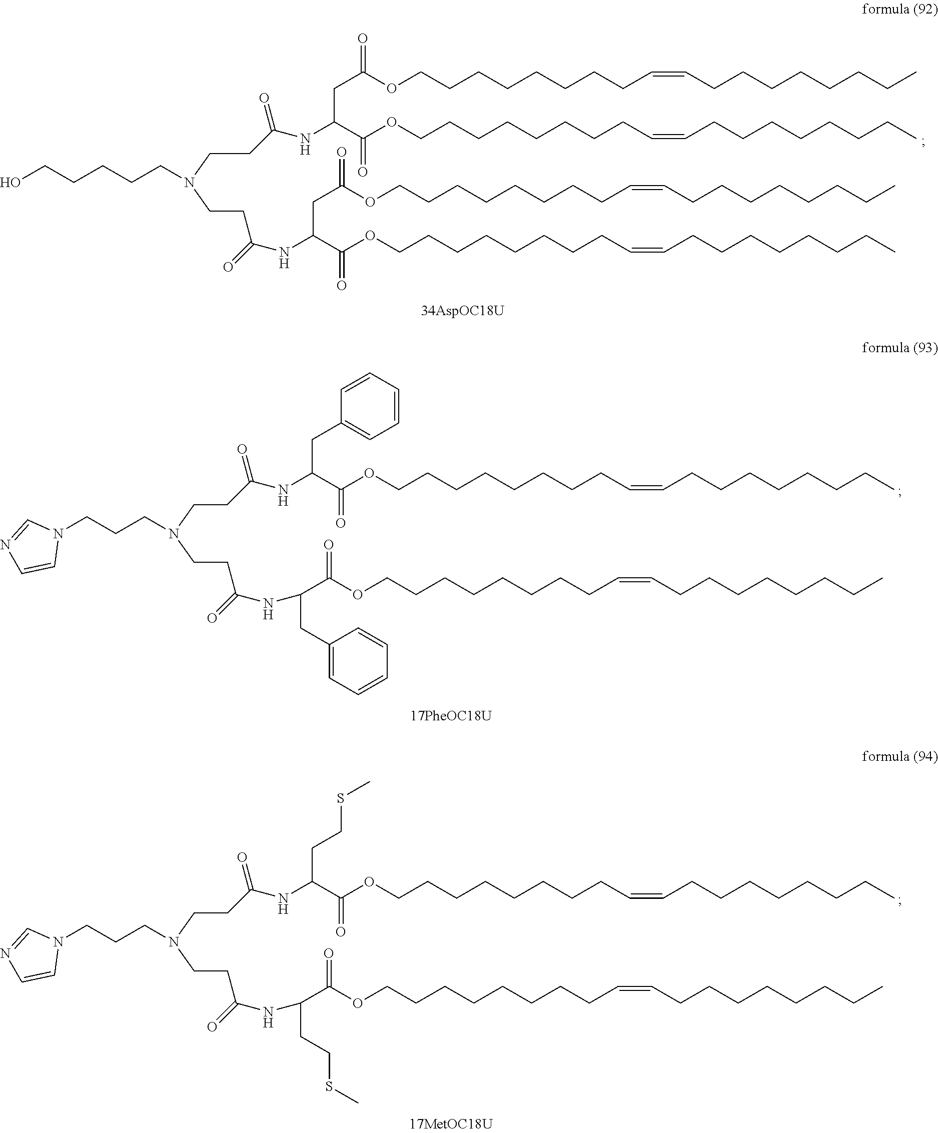 [SMILES: CCCCCCCC/C=C\CCCCCCCCOC(=O)CC(NC(=O)CCN(CCCCCO)CCC(=O)NC(CC(=O)OCCCCCCCC/C=C\CCCCCCCC)C(=O)OCCCCCCCC/C=C\CCCCCCCC)C(=O)OCCCCCCCC/C=C\CCCCCCCC ; CCCCCCCC/C=C\CCCCCCCCOC(=O)C(Cc1ccccc1)NC(=O)CCN(CCCn1ccnc1)CCC(=O)NC(Cc1ccccc1)C(=O)OCCCCCCCC/C=C\CCCCCCCC ; CCCCCCCC/C=C\CCCCCCCCOC(=O)C(CCSC)NC(=O)CCN(CCCn1ccnc1)CCC(=O)NC(CCSC)C(=O)OCCCCCCCC/C=C\CCCCCCCC]

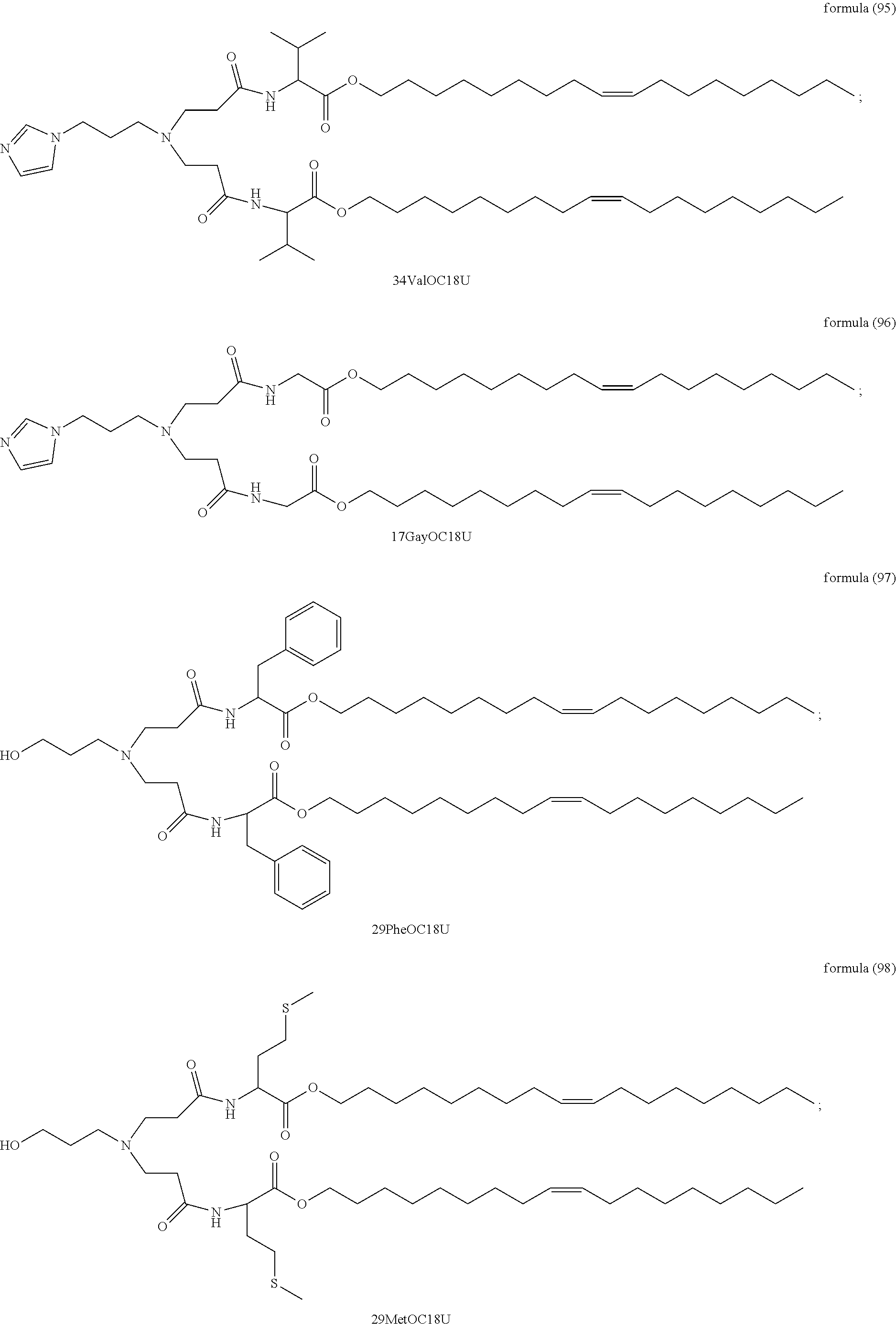 [SMILES: CCCCCCCC/C=C\CCCCCCCCOC(=O)C(NC(=O)CCN(CCCn1ccnc1)CCC(=O)NC(C(=O)OCCCCCCCC/C=C\CCCCCCCC)C(C)C)C(C)C ; CCCCCCCC/C=C\CCCCCCCCOC(=O)CNC(=O)CCN(CCCn1ccnc1)CCC(=O)NCC(=O)OCCCCCCCC/C=C\CCCCCCCC ; CCCCCCCC/C=C\CCCCCCCCOC(=O)C(Cc1ccccc1)NC(=O)CCN(CCCO)CCC(=O)NC(Cc1ccccc1)C(=O)OCCCCCCCC/C=C\CCCCCCCC ; CCCCCCCC/C=C\CCCCCCCCOC(=O)C(CCSC)NC(=O)CCN(CCCO)CCC(=O)NC(CCSC)C(=O)OCCCCCCCC/C=C\CCCCCCCC]

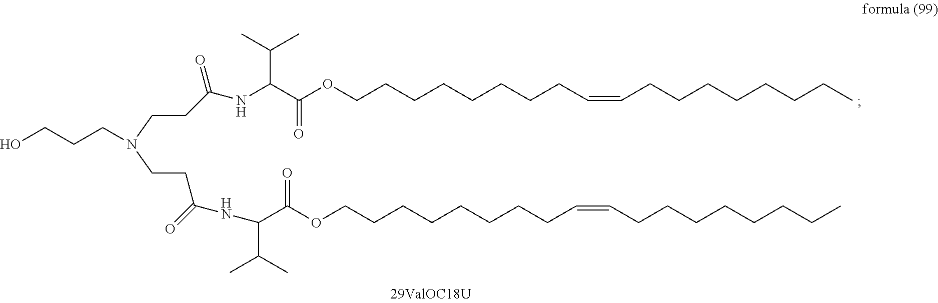 [SMILES: CCCCCCCC/C=C\CCCCCCCCOC(=O)C(NC(=O)CCN(CCCO)CCC(=O)NC(C(=O)OCCCCCCCC/C=C\CCCCCCCC)C(C)C)C(C)C]

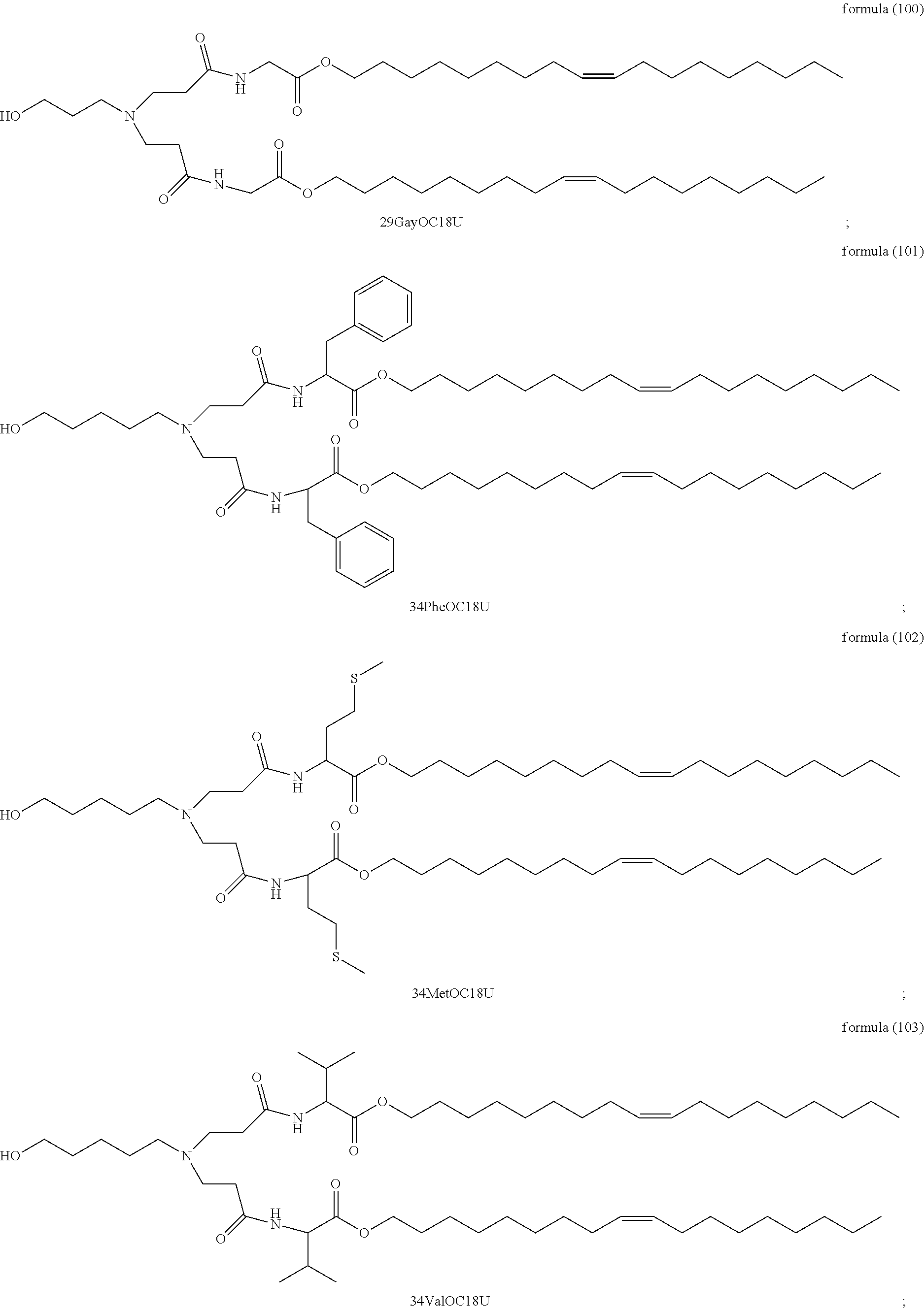 [SMILES: CCCCCCCC/C=C\CCCCCCCCOC(=O)CNC(=O)CCN(CCCO)CCC(=O)NCC(=O)OCCCCCCCC/C=C\CCCCCCCC ; CCCCCCCC/C=C\CCCCCCCCOC(=O)C(Cc1ccccc1)NC(=O)CCN(CCCCCO)CCC(=O)NC(Cc1ccccc1)C(=O)OCCCCCCCC/C=C\CCCCCCCC ; CCCCCCCC/C=C\CCCCCCCCOC(=O)C(CCSC)NC(=O)CCN(CCCCCO)CCC(=O)NC(CCSC)C(=O)OCCCCCCCC/C=C\CCCCCCCC ; CCCCCCCC/C=C\CCCCCCCCOC(=O)C(NC(=O)CCN(CCCCCO)CCC(=O)NC(C(=O)OCCCCCCCC/C=C\CCCCCCCC)C(C)C)C(C)C]

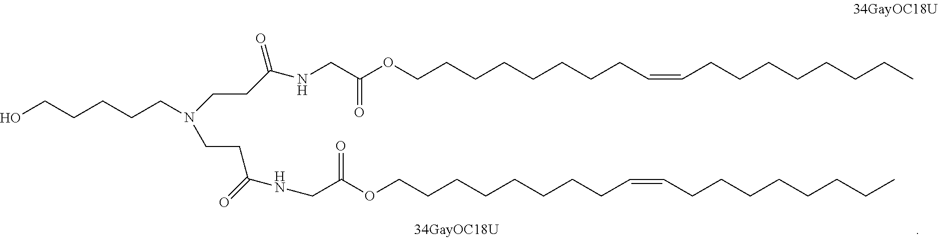 [SMILES: CCCCCCCC/C=C\CCCCCCCCOC(=O)CNC(=O)CCN(CCCCCO)CCC(=O)NCC(=O)OCCCCCCCC/C=C\CCCCCCCC]

3. A preparation method of the amino acid backbone ionizable lipid according to claim 1, comprising: tail compounds containing amino acid backbone or stereoisomers, tautomers, reacting with organic amine compounds through Michael addition reactions to produce the amino acid backbone ionizable lipid;
wherein a structural formula of tail compounds containing amino acid backbone is represented as formula (a): 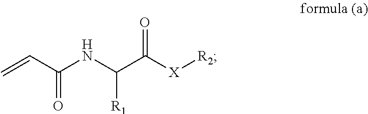
wherein R₁ is selected from 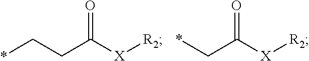
C2-C10 alkyl; C2-C10 heteroalkylene; arylalkyl or non-existence;
R₂ is selected from C3-C24 linear alkyl; C4-C24 linear alkenyl; 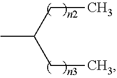
wherein n1=2-3, n2=3-24, n3=3-24;
X is O or NH.

4. The preparation method of the amino acid backbone ionizable lipid according to claim 3, wherein the organic amine compound is selected from:

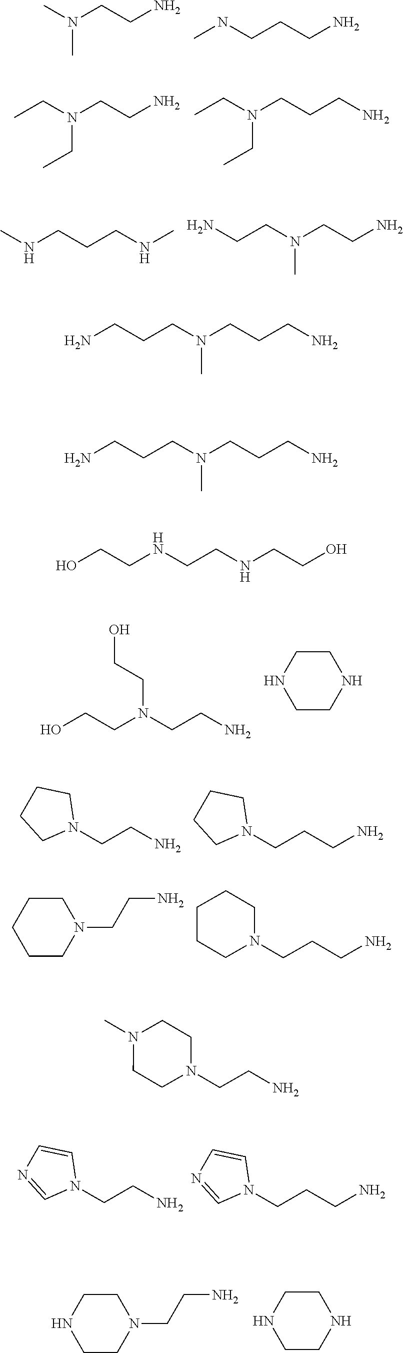

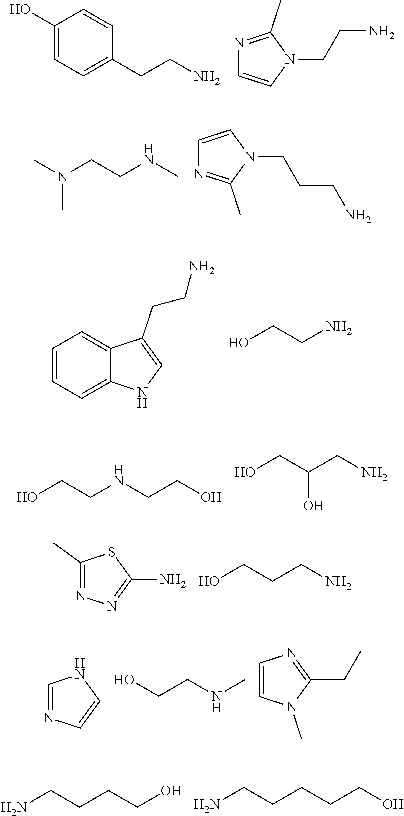

5. The preparation method of the amino acid backbone ionizable lipid according to claim 3, wherein a preparation method of tail compounds containing amino acid backbone comprises: preparing tail compounds containing amino acid backbone by esterification reaction between acryloyl chloride and compound a1

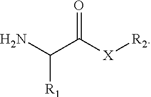

6. A lipid synthesis, comprising: the amino acid backbone ionizable lipid according to claim 1, sterol and PEGylated lipid.

7. An application method of the amino acid backbone ionizable lipid according to claim 1 in preparing a biologically active substance delivery system, wherein the biologically active substance delivery system is particles, nanoparticles, liposomes, lipid nanoparticles, or microbubbles.

8. A pharmaceutical synthesis, comprising the lipid synthesis and active substance; wherein the active substance comprises at least one of nucleic acid molecules, small molecule compounds, peptides, and proteins.

9. The pharmaceutical synthesis according to claim 8, wherein when an active ingredient of the pharmaceutical synthesis comprises nucleic acid molecules, an nitrogen to phosphorus ratio of the amino acid backbone ionizable lipid and nucleic acid molecules is (1-100):1.

10. An application method of the amino acid backbone ionizable lipid according to claim 1 in preparing nucleic acid drugs, gene vaccines, peptide or protein drugs, and small molecule drugs.
